SEQUENCE LISTING (14 sequences)

SEQ ID NO: 1 - protein, 1064 aa, Homo sapiens
MKIATVSVLLPLALCLIQDAASKNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKRRFSEENSKTDQNLGKAEEKTKVKREIVKLCSQYQNQAKNGILFCTRENDPIRGPDGKMHGNLCSMCQVYFQAENEEKKKAEARARNKRESGKATSYAELCNEYRKLVRNGKLACTRENDPIQGPDGKVHGNTCSMCEVFFQAEEEEKKKKEGESRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYQEQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 2 - DNA, 3195 bp, Homo sapiens
atgaagatagccacagtgtcagtgcttctgcccttggctctttgcctcatacaagatgctgccagtaagaatgaagatcaggaaatgtgccatgaatttcaggcatttatgaaaaatggaaaactgttctgtccccaggataagaaattttttcaaagtcttgatggaataatgttcatcaataaatgtgccacgtgcaaaatgatactggaaaaagaagcaaaatcacagaagagggccaggcatttagcaagagctcccaaggctactgccccaacagagctgaattgtgatgattttaaaaaaggagaaagagatggggattttatctgtcctgattattatgaagctgtttgtggcacagatgggaaaacatatgacaacagatgtgcactgtgtgctgagaatgcgaaaaccgggtcccaaattggtgtaaaaagtgaaggggaatgtaagagcagtaatccagagcaggatgtatgcagtgcttttcggccctttgttagagatggaagacttggatgcacaagggaaaatgatcctgttcttggtcctgatgggaagacgcatggcaataagtgtgcaatgtgtgctgagctgtttttaaaagaagctgaaaatgccaagcgagagggtgaaactagaattcgacgaaatgctgaaaaggatttttgcaaggaatatgaaaaacaagtgagaaatggaaggcttttttgtacacgggagagtgatccagtccgtggccctgacggcaggatgcatggcaacaaatgtgccctgtgtgctgaaattttcaagcggcgtttttcagaggaaaacagtaaaacagatcaaaatttgggaaaagctgaagaaaaaactaaagttaaaagagaaattgtgaaactctgcagtcaatatcaaaatcaggcaaagaatggaatacttttctgtaccagagaaaatgaccctattcgtggtccagatgggaaaatgcatggcaacttgtgttccatgtgtcaagtctacttccaagcagaaaatgaagaaaagaaaaaggctgaagcacgagctagaaacaaaagagaatctggaaaagcaacctcatatgcagagctttgcaatgaatatcgaaagcttgtgaggaacggaaaacttgcttgcaccagagagaacgatcctatccagggcccagatgggaaagtgcacggcaacacctgctccatgtgtgaggtcttcttccaagcagaagaagaagaaaagaaaaagaaggaaggcgaatcaagaaacaaaagacaatctaagagtacagcttcctttgaggagttgtgtagtgaataccgcaaatccaggaaaaacggacggcttttttgcaccagagagaatgaccccatccagggcccagatgggaaaatgcatggcaacacctgctccatgtgtgaggccttctttcaacaagaagaaagagcaagagcaaaggctaaaagagaagctgcaaaggaaatctgcagtgaatttcgggaccaagtgaggaatggaacacttatatgcaccagggagcataatcctgtccgtggaccagatggcaaaatgcatggaaacaagtgtgccatgtgtgccagtgtgttcaaacttgaagaagaagagaagaaaaatgataaagaagaaaaagggaaagttgaggctgaaaaagttaagagagaagcagttcaggagctgtgcagtgaatatcgtcattatgtgaggaatggacgactcccctgtaccagagagaatgatcctattgagggtctagatgggaaaatccacggcaacacctgctccatgtgtgaagccttcttccagcaagaagcaaaagaaaaagaaagagctgaacccagagcaaaagtcaaaagagaagctgaaaaggagacatgcgatgaatttcggagacttttgcaaaatggaaaacttttctgcacaagagaaaatgatcctgtgcgtggcccagatggcaagacccatggcaacaagtgtgccatgtgtaaggcagtcttccagaaagaaaatgaggaaagaaagaggaaagaagaggaagatcagagaaatgctgcaggacatggttccagtggtggtggaggaggaaacactcaggacgaatgtgctgagtatcaggaacaaatgaaaaatggaagactcagctgtactcgggagagtgatcctgtacgtgatgctgatggcaaatcgtacaacaatcagtgtaccatgtgtaaagcaaaattggaaagagaagcagagagaaaaaatgagtattctcgctccagatcaaatgggactggatcagaatcagggaaggatacatgtgatgagtttagaagccaaatgaaaaatggaaaacttatctgcactcgagaaagtgaccctgtccggggtccagatggcaagacacatggtaataagtgtactatgtgtaaggaaaaactggaaagggaagcagctgaaaaaaaaaagaaagaggatgaagacaggagcaatacaggagaaaggagcaatacaggagaaaggagcaatgacaaagaggatctgtgtcgtgaatttcgaagcatgcagagaaatggaaagcttatctgcaccagagaaaataaccctgttcgaggcccatatggcaagatgcacatcaataaatgtgctatgtgtcagagcatctttgatcgagaagctaatgaaagaaaaaagaaagatgaagagaaatcaagtagcaagccctcaaataatgcaaaggatgagtgcagtgaatttcgaaactatataaggaacaatgaactcatctgccctagagagaatgacccagtgcacggtgctgatggaaagttctatacaaacaagtgctacatgtgcagagctgtctttctaacagaagctttggaaagggcaaagcttcaagaaaagccatcccatgttagagcttctcaagaggaagacagcccagactctttcagttctctggattctgagatgtgcaaagactaccgagtattgcccaggataggctatctttgtccaaaggatttaaagcctgtctgtggtgacgatggccaaacctacaacaatccttgcatgctctgtcatgaaaacctgatacgccaaacaaatacacacatccgcagtacagggaagtgtgaggagagcagcaccccaggaaccaccgcagccagcatgcccccgtctgacgaatga

SEQ ID NO: 3 - DNA, 22509 bp, Homo sapiens
gtagattattcaaaaataagtgttggctgggcccagtggctcatgtctgtaaatcccagcactttggaaggccaaggcgggcgaatcatgaggtcaggagtttgtgaccagcctggccaacatagtgaaaccctgtgtctactgaaaatacaaaaattagccagtcatggtggcaagcgcctgtagtcccagctactcaggaatctgaggtgggagaatcacttgaatccgggaggcagaggttgcagtgagccaagatggtgccactgcactctagcctgggctgcagagcgagacaccgtctcaaaaaaaaaaaaaaattgttacttggctttttctatcagtacctatatcccctctaagtaacatatcagagtcccttctttcagaattgactgtctgatcaccaagataatgaacaaagctttgaagctaattctggttattaagcagagaaaagagtttctttcttcacttctctattaacatttcaaatgggatgatttttttttttcggggataagggtttgtcctgaatattgtagggtgtttagcagcatccctggccactatccaccagatcccatagcaccttcacagttgtgacaaccaaaaatgtctctagacgttgccagatgtcccctgaagggcaaaattgccgctagctgagaggcaataaaatggagcaaagctaagaaaaatgcaaactggaggttaaacccatgatataaaatgctgtatatgagatgagtgtgtttgtcccctggaattcagggtgatgaaacgagtcaatccccaaaatcggacatggtggaagattgaaaaagaagctgtggtttgggaaaaccacctaggaccgggagtcaggagaatatgaatgagttcaagttcttccactttctagctattcttgaatcaatgacttccccttgccagagcttcttaataaaatgaaaggatttaaatatgctctactagaatatactgcagcccattctaggtcaaaggctatggtaagtttgtgactcttcataaatctatacttggttctttgggtactgaactctctctagaactagactctcaagaaggctgagtgtgttttcttgtcagagcccaactaacaggttttctgtctttcttttttccttgtctcacaaggttcatttctatatccatcctcactacttaaagagaaatggaggtagaaagtgtgatctggcaaagtcagctcttttgccctaaaatgatagtcatttttccccttggaaaagaggccttaggctaaacagtttctctggagtaagcactatttttttgagctcagaaatataacctaaacgggcaaggaatcaagagtaaacttccatgggaattgtagagcccaggtctctacatacgcctctcttagaaaacggacgtcagaggatattcactgtttcctccataagtaggctgccattttcccattgcatcacagctttaagatgagagattactttgactcccaagctttgttttccagttcagcttcatgcataccagctaattaggcaacatgacaacagggtggagcgatacttgtattctggatgtttccacacccacagtggatttggcctggtgccgctgccagccaccctcagagcagtggcatcttaaaatgcaaccacacccagacaccaacaaattgtattttttaaagattcatgttaatgaaaaattgtatattattgattaatctgtaggcaaaaaagaaaagtgtcagctctttcttcccttttctcatcctctaccttttttgaaatttacagatacaatgagctagccatgtaggtgagtatacgtggtgaaggcaggagttgaggcggcttgacccacacaatgccaatttggactaacccaggcttttggcatatcaggaacacgggctagcttttgcttggcgccttagagcttgtggagtacgttcatttacagtttaattatattttcatgacaaaactgatatataggtggtattatttatattttataaataagaaaaatgggctcagatataaataaacgtgtcaaaaatcacacacaagttgaggggcccagttttgaccccaaatctcattcttttgtcactcttttcatagctattttctatgtaacttaatggtggaatgttctggtctaagatgtcatcaattaattctgtatatattgagtgctattatgttccaggtactgctacctgcagaggcaatatttttattacattggctgatatactcgacaagtacggtttaattggtctgccttctttttgtcttcttgtttcatttcccagtgtgcctcacctcatgagtaatttaggaatgtaatctgtgctcacagtcttgcttcagctaaatgtgagggctggaattaaaacacagacttcctaactccataacaaatatcattttgaatacaatcatgaccttgctagaaagttagagaaataagagggattgataacagcatgaaaggacattttttttttcaataagacatttcttctctttaaagaagagaagatgtttgaacatttaagagctttaaatgagctgtttggttttctgaactatttgaaaagttgtcattttttcatgactcaagtgacctgtcaatccttccagccacgtgtggctattgttcattgaaggtgaaccaattccttccaaagacgtcaccatttgtaatacaataaactgttcaaaggaaggggcactaacaagtaaatgatttctctaccctggaggttactaatttgtacccttaaaattaggcacgttttttggcacttcccacaaaatattcagcacttcaagaaacttagaggtgaattgccccactatgatctcttgtttgatgtaatcaacaggctaacaattaaaagacttctaatgatagctaacattgagcattccccatgtgccaagtatcatctaaaggctttacaacatgaatgaattaacttaatccttgtgacaatcatgattaccacctctcagtttagtggtgaagaaattgagatagagggtttaggcaaattgcccatcctcatacagcaaagaaggcagaatttgacccctgtagcattgctccaaagccaaacactgaatttctgaattatgcttccttctcaagaaggcttggctatgtatagcttgactgttaccaataatagatgcttcagctctgtaatgtaatcatcccgatctgctcaatggtcttatcaaggggatgatactcatggtgatttgcctgaggtcaaagtgttagcttgtcacacatttccaaaatgtcactggtctctgtatggtgcagactggagaagtgatctctgttgacgcactgtctgggtcagtctggagaagacagtccaggaaaatcccgaaatacagtcatacctcagaatagaccaggattgatttccaggacccccgcacataccaaaatctgcaggttcttaagtcctgtagctaccctactcaatttgatgtgataagtgagtccttggtataggcggttttgcatccttcaaatactgtattttcggatatgcctttcggtgaaaaaactctgtgtataaatggacctgcgcagttcaaacctaggttgttcacaagtcaactgtactttactttggaattacggtgtgggtgttttaaaaggataacgatcacgcttcctctccatcaaaataaatcatgacgtttgtagttgaatcatatcagggaaaggtgtcattgagggtgtaggctgcttatttttaaacttgaaataactaggaaatgcactctatttgtcttagccctcctatcttcgtctgtaaattaaaggaaggaggtgtgctagatgaatttcagtattctatccagcccaaaagatatatattcacttagttaaaaaaaattcatttttgttcttaaaaataatacctaatttgtgcagaaaatttatcaggcaagaaaagtaggaagagaaacattaaaaagtcataagtaattccacccatcagagataacaaatgttaagatttttttgaaaacccatttctgtctcccttaacacagacatatataatgctttaaaaatagtatgaaatgaatatgatttttttttctaaaattaggtttactgagatacattttataaaagcaaaattcacagtttgggtgtgcagttacatgacttttggtagacaaaatcacttatgcaaccaccagcagaatgaagatatagagtattttcgtcacccccaaatctcccatgtgtacctatgtagtcagtcccctcccctgatctccagcaaatggcaatcactgttctgatttctgcccctaaaatttgtccatttacaaatgtcctataaatggaatcctaaagtatgtaacattttgtttcaggcttctttcactaagaataattcttttgaagttcatccatgttgtagcaggtatagtatttcttttttaaaattcttgagtagaaattcatcatgtagatgtgccagttggttgacctattcatcagttgttgtacatttggcttgtttacagtgtttagtgattctgaatcaggtcattataaatattctcttatgggttttgtatagacatatgttttaatttctcttgaataaataagtagtgttagattgctaggtcatatagtaagtgtaggtaggattttaaaatcctcttttgtaaaaacttgttttaaaaaatattttgtttcataatttaaaaaaatatttgttttaaaaaagatttacaggaatttggaagatggtacaacaatgttctgtgtacactttacccagtttcctccaatagttaagtctttttttctttagattttctttgtttctaattcatttgactcacaaaataaatgggtgctaactacagcagttatagtgatattattcacactacacagaagaaaccttgggtcattaattgataacatcaaaacctcattcacctacattgaacttccagaatcttctgtaccaactgcctaaaatactaaaaatgtagaatactacaaattaaagatgaataccttggtaggcatgaatgaccgaagtaaaaccaagatttatagagagtcaattaattaaaaatcttccacaaagagaagcccacagccacatggcttaactgacaaattctattaaatatttagtgaagaattaatgtcaactcgtcacaaacccttccaaaaaatagaggaatactattgggaacacctaccaattgattctgtgaggctagtattaccctaatactaaagccacacaaaagcatcacaagtagacaagtatgaataaatatccctgataaacacacacccagaaatgcttaaaaatgctagtgaaatgagtctagcaacatataaaaatgactgtacaccatgaccaaaaaggattattttatgaatgcaatgttgacttagcctccaaaaaccaatcaatgcaatacacataactaataggataaagaacaacaacaaaaacatcaaaaacaacaaaaagatctcttcaatagacacaaaagagtgtttgaccaatccaacacccatttgtgattaattaatctaaaaagcttctgcacagcaaaagaaactatcatcagtgtgaacaggcaacctacagaatatgagaaaatttttccaatctatccatctgacaaaggtataatatccagaatctacaaggaacttaaacaaatttacaaggaaaaaaaaacaaacaactccatcaaaaagtgggtgaaggatatgaacagacacttctcaaaagaagacatttatgtgacaagcaaacatgaaaaaaagctcatcatcaccggtcattagagaaattcaaatcaaaaccacaatgagatgccatctcatgccagttagaatggcgatcattaaaaagtcaggaaacaacagatgctggtgaggatgtagagaaacaggaatgcttttacactgttggtgggagtgtaaattagttcaaccattgtggaagacagtgtggtgattcctcaaggatctagaaccagaaatatcatttgacgcagcaaccccattactgggtatataccaaaggattataaatcattctactataaagacacatgcacatgtatgtttattgcagtgctattcacaataggaaagacttggaaccaacccaaatgcccatcaatgacagattggataaagaaaatgtggcacatatacaccgtggaatactatgcagccacaagaaaggatgagttcatgtcctttgcaggtacatggatgaagctggaaaccattattctcagtaaactaacacaggaacagaaaaccaaacactgcattttccacaatggttgaactaaagtaccctcccaccaacagtgggtaaagaatatgaacaaacacttctcaaaataagacatttatgtggccaacaaacatatgaaaaaaagttcatcattactggtcattagagaaatgcaaatcaaaaccacaatgagatactatctcgtgccagttagaatggcaatcattaaaaagtcaggaaacaacagatgctggagaggatctggagaaataggatgtggcaaatatacaccatggaatgctatgcagccataaaagaggatgagttcatgtccttcccagggacatggatgaagctggaaaccatcattctcagcaaactagcacaggaacagaaaaccaaacactgcatgttctcacttataagtgggagttgaacaatgagaacacaggaacagaaaaccaaacatcacacgttctcactcataagtgggagctgaacaatgagaacgcatgaactcagggaggggaacaacacacattggggcttgtcaggttgggggtgttgggggcaaggggagggagagcattagtacaaatacctactgcatgcggggcttaaaacctagatgacgggttgatgggtgcagcaaaccaccatggcacacgtatacctatgtaacaaacccgcacgttctgcacatgtatcacagaacttaaagtttaataaaattttttttaaaaatacaaaaattagctgggcgtggtggtgcacccctgtgatcccagctactcaggaggctgagacacgagaatcacttgaacctgggaggcagaggttgcagtgagccaagaacatgccactgcactccagcttgggcaacagaatgagaccctgtctcaaaaaaaaattgtgtagtttaaaatccacttattctatcatagaaccagatagtagcagctcatttaaaggatctgtaaatataagctgtaatttttctgccatctccagatgaacacttccttgatttccagtcagattttactgttagagagctgtccctttcaatggagtcatacctgtctctaatttcaatcaggttctacctgtgcattcttgggttatatgaagtagaactaactgttgttgttgttgttttctgcctgatcctaaagaatttcaagcctttcctaagtgttcaaagtgccaatgggtaacagttctggattaaagttgattataaacagattctatgaattctctatatattatgtgaatactttattttcatcaaggaaaacattctctcatccaatttttgtaattcgtggctatctctcatttattaggcacaaaccacttagaattggaatgtcctctccgtgaaatgattctttgtcgtcacgaaatgattctttatccctggtgatattctttgctctggaatctactttgtctgatattaacataggcactcaagcttttttttttatttgtgtaggtatcatatattttatcccattcttttacttttgaactatttttgtatttatatttaaagtatgtctctttaggcagcaaatagtttgggcttcctttttaaaaaatctaatctgtcaatctctatcttttaatttacatatataacctatatctatgtataatacatttacataaagtatattcttacatttaatatatacataatttacataaaacagatttacatatttcataatccgcatggtcagatttgcatctaccatcttattatctttaccaattgttttctatttttatcacctgctttctatttttactaccttttttttctgagacaggatctcgctcttttgcccaagctgtagtgtagtggcatgatcctggctcactgtagcctcgacctctctaggctcaggtgatcctcccacctcagcctcccaagttactggggctgtaggcatgcaccaccattcctggctaatttttctatcttttgtacagatgggatttcaccatgatgcccaggctgctaccacctgtgttttaattccccttttcttcttttctgctttctcttggatgaatcaagtattatgatatcattgtatcttcttttgttaatttactaactataaatctttggttgttgttgttattgtttaaacagttggttattttcaaaagagatttaaataataaaaaaatatatttatccatgtagctactctttttagtgatttcctttcctttgcatagattcatatttctatctaatatattttgtcttctgcctgcctgaaaaatcttcctttaacatttcttctagtgcaggtctgcagtaagtgctgaattcttttaactcttttatgtaaaatcgtctttatttcaccctcattttcaaaagttgatttcatgggacatagaattctagctggtagttttttccttcagtcacttaaagatatagtttcatttcctcttcacctaacattgtttattttttttaatctggtattatcttattctttactcctttgtatgtaatgggatttctccctcacctctctcacccatctaccagctgattttaagacttcttgtctcaatatttttgaacaatttgataatggtgtgccttgatatagtttacacacacacacacacacacacacacacacacacacacacattgtgcttaggattcttttaaattcttggacctcttagtttatagtttcttggacctcttagtttatagtttcttggacctcttagtttatagtttgaaacaaattttggctgttattacttctaatattttttttttacttcccaagacccactttcagggactataattatattagtctgcttacaattatcccacagcttgctgttggtcttatctttttttttctgttttattttggacagttactattgtacaccttcaagtttattaatcttttcttcaacaaggtcaaattattgttaattacatttagtatatttttaatctcagattcagattatgttttgtttttattccttgtctctgctttacatttttgaatttatgaaacagttataataactgttttaatgttcttaagtataattctaacatccgtaacagttttgagtcatttatcctacttgtgaatcatatgtccttgcttcttggtatgcctagtaattttagactgagtgccagacattgctaattttacattattgagtagtgaatgctttttgtatttctgtaaatataaagttttgccctgggacacagataagttacttggaaaccatataatctttttgggtcttgctgttaaatgttttaaagtagaacctatggctaattatcccctactactgagccgcagcctggtcttctgggaatcagagctgtgtgcctgtgcctgagcttcttctccgtgctctatggcttagacattttctcaggccagtaaactggagcaattttagggtttagcttgtttgtttactatctcgtgtatgacttttcttcatttcaaaagactgatgtccacaaccttgaagatcattgtttaatatattgtgtatttgtatttgttgttgttgtttgtttgctgcaggcaggagtgtaaccccagtccctgttactttattttggctagaagaagaagacatccagacagttttttcctattgattttgcctcttattatgggattattttcctgtttctttgtatgtctggtattttttaattttctgctagacattgtgatttactttgttaggtgatggatattttttgtattcctgtaaatatctttgaactttgtcctgggacacaattaagttacttggaaatagtttgcccattttaagcttgtttttaagctttgttaggtggaaccagaataggctttgaggttaattttgccccactactggggcaacacatctctctaatctgtaaatgaatggcaaggtttttctttctgacttacaggaacacaagttatctcctcccatatgtaagttctggaaatcattccttctaatccttagagtgattctttctgcagcctggagtagttttctcatgtacatttacctgtcagtactcaactgaagattctaaataaactatgtgccacaaatctccaaatgtttctctttgaatctttctcttcttgttaacatgccacggaaattttacctcctttggtctcacagaccctcaggtttgtatgatcatctcagggaagcttctattagctacctgtgttctccctctccgtgctacagcctggcaactttctacaggctgtgaactgtggtaatgtagggataacatggtttgtttccccctcccaagaatcagtgcactgtattatatcatgtccagtgtcaaaaacaattgcttttgcttttatttttttctcattgtttcaggtgaaagaataaaatagtttctgttattccatttatgccagaagcagaaatcatatttataatttaaaaataaactgatgtattccttaacccacacctcaatttgtgtttctattttcaggatgtaaagccggcatattaatattctcatttccccaattaaagccttcacattatttgattcctaataaaattataaaagctatgtcttcaagcagctttaacattgttaagaaaggtgaaggacataaggaaaagacagaatcttgatttaatagacttctatctgattctgattatcaccacttattatttgtgtctatggggaagctcctatatctctctaagtttcagtaaattgagaatgataacaatgatacatgtccagcctattctacaggctgatcatgaggacaaatgagaatgcatacagaaaaacacggataatttgtaagcattttacaagtgttagtatttaccttcatcaccaagactagtttaagttcattttttgctttcaatccattgatgtctttcaattctctttatttttctaaagatatacctctcctttggactcattccatattttccttttctttcatatcctagtgtcactgagttggtgtttaaaactctgttgaatatgaaaaaaatatatcagaacatgagtcacgaggcctgagttcctgtctcagctcttccatttatggctaatagatttgagacaaattagtcaatcatgcagatctttaaacttctcatccatgaaatggaaataacaccttctctctccttaatttaaaggtgaggatcagctatataaaataaacagaatgtaaatatttaaatattaatacacacatattgtgtgcaaactggtattatacaagttattctataactatgcactattaggcaataaattatacataaaattgctctataataatataacaaacaaatgcctttagtaatgctaataactgtaagaagtacttaaataattttctatacagtttttaagtacttttcccagcagcataacaagtattttttatctagaagagacagaaaaaatagtccctgtactgtagacaaaagacagttattactttttccagggttgtttatgtggatggcaaataaatgtccactatgtatagtttcttgctcagaaatatgaaaattaaaatttgacctctcagaggtcagagattctatctgttttttttagaagtttaacattgggctctaacacagtgctaggcaaatgcaggcatttagttaatatttgtcatattcacctggagagtattatggagctcatctggaataccataataatttcttaaccttgggtttagatttttaaatttttaaatcacattttagtgagttggtcccacgcctgagaattccctgattttagagttattactcattcgcttcttcctttttttttctgtctattgaatcaaggttgtggtgtgacacaatggttagtgctgtttaacttggtacattcttaatttttaaaccttacaacatggatttgagtctcagcttgaccgcttacatgccatgtggcctgctgtgagtcactgagcctctctacgtttttgttaccttccttttaaaccagccttctttcttaaagcaactgagatgaaataaaacagtaattgtggaaatttgttgtagattataaagcggaattcatctttttgggtttttttactcattcatttattgatttcactaaaaatatcatttatctaattggagagatagacaagtcaactgacaatttcaagactgtgtcatgagtgcaaggtgtggaagtagtaatggcaccgggtactgaggggcaaagagtaagaacactctattaagggtcaggaagggagcctctgggaacacacagtctaagcggaatcctacaagcaaagatatagaagtaaaggaaaagatcatcctacaaaacaaagaagcaatatgtgaatgctttagtaatgggaaacgttatcaagtaaacatttttggtgaacggtatttgggttaaagcaagagaagtctggaagtctgtctagctctgttacacacattgctggaggacatcaaaatgtgtgcattaaagcccttgagagtacaaaagataaaatctatacaaaggagccaaaagactgataacaaatgcaatatgtgaactccaacctgaaagtttgttaaccagttggctttctactttatctaatggcaaagattatttcttcgaccaaactttaatcaaattcctctgaaccctctttttgactgggccttaaccttggcctatgagaactgcaaattcttagcccaaatgatgatgtctacctccctttgtactaagaggcttgaacaaatgttaccacagtttctaatacctcaagaacatgttcctgatgacccagcccctgcttaagttcctatctgaaaagctcaatgctacctaaataatttactatttacctgactgtagacccatgacctcccatttcttagagcatttactttagaaaacttgcatctataaattagcaaacacaaatggcctaaccacaatgaccaaccttccctgacatctcctttagtacttttcctttagcacatgatatggtttgggtctgtgtcaccaccacatctcatgtagctccgataattcccatgtattgtgggagggacctggtgggagatgactgaatcatgggggcgagtctttccagtgctgttcttgtgatagtgaataagtctcacaggatctgatggttatagaagtaggagtttccctgcacaagctctctttgcctgctgccatccatgtaagatgtgagttgctccttcttgccttccaccatgattgtgaagcctccccaggcatgtggaactgtaaatccaataaacatctttcttttgtaaatgcccagtcttgggtatgtctttatcagcagcatgaaaacggactaatacagtaaattggtaccaggagtggggtgtttctgaaaagatacccaaaaccgtggaagcagttttggaacttggtaacagacagaggttggaacagtttggatggtccagaagaagacaagaaaatgtgggaaagtttggaattccctagagacttgttaaatggccttgacaaaaatgctaatagtgatgtgaatgataaggttcaggctgaggtggtctcagatggagatgaggaacttgttgggaactggggcaaagatgactcttcttatgttttaacaaagagactggtggcattttgcccctgccctagagatttgtggaactttaaacttcagagagatgatttagggtatctggcagaagaaatttctaagcagcagagcattcaagagataacttgggtgctcttaaaggcattcagctttttaagggaagcagaacataaaagtttggaaaatttgcagcctgacagtgtgataaaaaaaaagaaaatccattttctgaggagaaattcaagccagttgcataaatttgcataagtagcaaggagcctgatgttaatccccaagacaatgggaaaatgtctccaagagatatcagagacctttgtggcagcccctcccattacatgcccagaggtttaggaggaaaaaatggttctctaggccaggcttagggtccctctgctgtgtgcagccctgcgttgaagacactccagtggctgaagggggccatggcttgggctatggcttccgagggtgcaagcctgaagccttgacagcttccacatggtgttgagcctgtgagtgcacagaagtcaagaactgaggtttgaaaacctcagcctagatctcagatatatggaaatgcctggatgtccaggcagaagttttctgtaggggtgaggtcctcatggagaacctctgctagggcagtacagaagagaaatgtggagtaggagcccccacacagagtccctactgggcaccacttggtggagctgttagaagagagccttcatcctccagaacccagaatagcagatccacaggtagcttgcaccatatgcctgagaaagccacaaacactcagtgccagcctgtgaaggcagctgggagggaggcggtaccctgcaaagccacagaggtggagttgcccaagaccatgggaacccacctcttgcatcagcatgacctggatatgagacatagattcaaaatatatcattttggaactttaagatttgactgccttcctgaattttcgacttgcatggcatctgtagcccctttgttttggccaatatctcccatttggaacagctgtattgacccaatggctgtacccccattgtatctagaaagtaactaacttgcttttgattttacaggatcataggtggaagagacttgccttgtctcagagtgtggacttttaagttaatgctgaaatgagttgagactttgaggcactgttgggaaggcatgatcaggtttgaaatgtgaagatatgagatttgggaggagccaggggtagaatgatgtagtttggctctgtgttcccacacaaatctcatcttgtagctcccataattctcatgtgttgtgggagagacctagtgggagatgactgaatcatggaggtgggtcttttctgtgctgttctcatgataatgaatgagtctcttgagatctgattgttttaaaaatgggagtttctctgcacaatctctctttgactgctacaaagatgtgacttgctcctccttgccttccaccatgattgtgaggcctccccagccacgtggaactatgaatccaatacacctctttcttttgtaaatttcccagtgtcgggtatgtctttatcagcagcatgaaaatgaactaatacagcacacctcagcattaaaaaagcatcctgcctctggttttagcagaattgagcttggtttctactggagtttcttttctctattggaatagcccagataaagtgagtcttgccacttttaataaatattcagctctgtttctctttaacactattctacaggatcataagtttaatgtactggttacttaaagagaaaggagcctacataagactgtattttcaaccgtataactgtgtctatacatttttacacagtttgcatgtgactgggtcaatacatacggtgagtgtattgtttgctatggctgccgccacaaaataccacagactgggtgacttaaacaacataaatttattatattttcacatttctggaggctagaatttcaagatcaaagtgacagcaagtttggtttcctctgaagcttccttcttggtttgtaatgggcaagttcccactgtgtcctcatgtggtttttcctctgtgtgtgcatcactagtgtctcttttgtgtgtccaaatttcctcttctagtaaggacatcaatcagtttggtctaaggcctaaccttataacctcattttaacttaaccaccacttttacttatctccaaatataggcacaccctgaagtagtggggtttaaggcttcaacatataaattttgggcagacagagtttagctcttaacattgagtacctgagacacattagttacagttgtaggcaatgtggttactacggtgtgtagtgtttaagaagctgggctctggagtttgaatgcctttattacattgactgtgatcatttgcaagatcactgcttacaagctgtatgaccttggaaaagttatagaatctcaccaaacctcagttgctttacctgtaagaataaaatgatacttaatctttatatttattgttgtacctaagtgcatgaaatgtgttagcaaattttatttctttacaattttttttttttttgagttggagtctttctctgtcacccaggctggagtgcagtggcgtgatcttggctcactgcaagctccgcctcctgggttcatgccattctcctgcctcagcctcctgagtagctgggactacaggtgcccgccaccacgcctggctaattttattattgttagtagagacgggtttttcttttagtagagacggggtttcaccatgttaggcaggatggtctcgatctcctgacctcgtgatctgcctgcctcggcctcccaaagtgctgggattacaggagtgagccacggtgcccggcctacaaattattttgtaaagataatctttttctaactttgctataaatctaacagtttcattcttccctactgtaaacacacacacacacacacacacatattacacatataaaatttgcaatgcttttttgctaaaattaggtagacttttttttttttttttgagaaagagtcttgctctgtcgcccaggcgggagtgcagtgaagtgatctcagctgactgcaagctccgcctcccaggttcacgccattctcctgcctcagcctcccgtgtagctggggctaaagacacccgccaccgtgcctggctaatttttttgtatttttagtagagacggggtttcaccgtgttagccagggtggtctcaatctcctgacctcgtgatccgcccatctcggcctcccaaagtgctgggattacaggcgtgagccaccacatccggccggtagactcttaggtacttcaggattaatttgttatgatttattgaaagactgtagctcctcaaacctctgttttctattgtcaggataaatcctaggagactttaaaatgcactaaatttttgagtgataaacagtgtggggccatgggtcaggggttcaagtaatatcagctatagggagaagctgattgttgtttctaaaataaaatataacccatatgaaaatattaatacaattaaaaattagacattgaaattcctgtcttcttaatcctaatctaataaaagcagtaattatgtctgtaaatagcagggtctatatactcagtaaacaaacaatgcccatcccattctattattagataaaatgagaggcttatctctagtttcttagtctttccagacaaacagaaaggaattcctacagagagagtgaagttagttggtaatgcaacattaccagataagaagatgagtaaaggctgttttccagttagtgtcttttacaaaggagaatataggccgggcgcggtggctcatgcctgtaatcccagcagtttggaaggccaaggcgggtggatcacgagttcaggagatacgagaccatcctggctaacacggtgaaaccctgtctctcctaaaaatacaaaaaaaaaaaaaaaaaaaaaaaaattagccaggcacggtggtgggcgcctgtagtcccagctattccgggaggctgaggcaggagaatggcatgaacgctggaggcggagcttgcagtaagccgaaattgcaccactacactccagcctgggggacagagtgagattccgcctcaaaacaaacaaacaaacaggagaatatagacatgttagattgatgggggaaaatattagaattatagaaaagaaaataaaaaaataaaaaagaaattatttcatcttttaagatatattagaatatataaattttactcatggtcacaacatacctctttttgcccaagtgattgtgtatgtgtgtctgtgtgtgtgtgcatgcatagggctgtatctagtttaaagcactgtaacgtctggattctctttttttttttgagatggagtctcgctgtctcccaggctggagtgaagtggcgcgatctcggctcactgcaagctccgcctcccgggttcacgccattctcctgcctcagcctccggagtagctgggactacaggcgccctctgccacgcccggctaatttttatgttttcggatttttagtggagacggggtttcaccatgttagccaggatggtctcgatctggattctctttgaacggtataaaagtctgtgagatggccgtggggaaactcagagaaattaaaggtataaaatgtaagaaagtgtcaaggctgagatgaccatcccagctagttcctactgcaggtttcttttatgacaccaattttggctggtaaagaaaagataataacacctaattggctgcctcacaacactgcataaacaaatcaaaagagctaatgaatggcaggttgttttgaaagctgtggaaggtgatctaatcataaaagactatagttatttattaactattatcgtcatcaaggctccctctgtgtagaaaaacagaagactttaggaatgatatggtaactttatattttggaaataacctttgtttgtagtaacattactgatctacaacaatctttataattttctcatttcctccccctgtatcagtatccttcagattctgccaacctcataaatgcgtaaggttgtaaaattcatagtttttattatctaagcccactcatttctaatgagaggaaaaaagtttctacatgtttgatgaataattgaaagagttaaaaacaaggatgatattttgagttagaagaataccttgataacctaactctgacccaaacctgagttctccaaaccttcaatcccattcatttaaatgtaatattcatggtgattttcaggaatgaggagtaattaacagagcatgtgatgaatcacctctggctttacaacattcctaaaatgaaatggagaagaagccctaatgaaatgagccagctgatgagtacttccaattttgggggtgctcactgaatcttctttttactactttctccatttggaaaagatatcagtttcaaactttttattcatcatttcagaaaaggctggcatctctgaggagctcttttccagtaagaaggaaaccaatgtcttcaatgtttcctcatagtagagattcctaagacaggcaatcatcttttggttcttctgttccaccctagcaccacttccactcctgggaatattctccatttgggctaggctaggggtcactagagcataatacacagtcattgattccattttaaggcatcaaactcttttaccgtcaataacagaatatatggttgggacacatttcgtatggtctgacagggcagatgaaagataaaaaattagtattgatatgtgaatatttagaagaggcttttagagaaagagacaagaaatcaatgctgattgaaacagagcaatggttttcgttaagttttaacaggtgatgtttcgatttgtttaggcattatggtttaaaatacaattaaaagggaaaaagagaacatatatcgttatagtttaatttcacctcaaattgtgttggggtagtttttctatgctgagtaaatataaatctattggtgaaatccacagtcaacagatgtttacttttcttggtatcttagaaattatctagtacaaccacgttattataattatgtaaaaatgcaagaccagctatcattaactatcaagcttatcacataaacttgataattgttctaagcacctctctctctcacacacacacaaacacacacgcattatatatatgatttatctctaaacctcttagcactagtacaatgtacgtgttattatttttattttacatattaggaaactgaggcttagagaaaataagtgacttgtccaaggcaacatggctaacaattcaagatttggacccatttctagcttgcttcaaagctgggttcatttctatccctacatagttgaaaagactacatagaatagaaacagatttctgaaaccctgtctcattctctttccattaagctatgatggactttgattttctaacctgtaacaataatggaatttgaacttgctttccgtggtcactaattttactattccattggaaaggagccaagctacaacaatagtgcagaccttgaaaccaatgattttatatatatatatatatgtgtgtgtatatatatgtatgtgtgtgtgtgtgtgtgtgtgtgtgtgtgtgtgtatatcttttttttttttgatggagttttgctctgtcgctgaggctggagtgcagtggcgccatctcaactcacacaccctccgcctcccgggttcaagcaattcccctgcctcagcctccagagtagctgggactacaggtgcgtaccaccaagcccatctaatttttgtattttaatagagatggggtttcaccatcttggccaggatggtctcgatctcctgacctcatgatccgccagccttggcctcccaaagtgctgggattacaggcgtgatacactgtgcctggcccagtgattatatttttataggaaataaattatttcatgagagaaaaaagaaaggacgtattatctcactgtgataaatctcataatattggcatactccctaagttgggcaactttgatcagttttctggaaccatcataaaaaggcctttaattggataaagtctcgaacttttcttctgtggatgtgaatatcttgagaagtactggaatgaaacaatttggctctggtttcagtcatcttcacatgtttcttcaggtgctttcttaagctattagggcaaaataagctggtagaaaaaaggtgttacttgtaggatgaaagaagcaactaaagagaagaaactttctctagaaaaatgttacatttacacatttataattgttaacaacttttttcttcttttctttttgggagactaaataatgggatgatacaggaagaaatatgatttagagaaaactgattgagccaaatttttcaaacggtaatatgggtaatcataggctatggaaagttaaactgagtctccaatgtttatgattatgatgtttggctaggatataaaataaggcttcagaatataaaatatcctgcagattgagaagttttaaaagtagttacatacaagttttatttttccaacgttcctgccttttctaatgtctaaggatgaagcagtgtacagagaggaaagaatgttatatgtgtggtctgcttgcaagtgctggatgcctcctctgcctgctccaactaaactgtctgcttatgagaaagtcattgctcctctgagttcctgcctccctatatattcaacattagaattttgccaggttatcttttagggtcgttcaactctaacatatatatgagtctgtgacactgaactcagacacttcagaacaggaaccctggatttctctggatgtggaaatctttaaatctatacctgactggtaggacaaccaatctgtcttcctttcctctcctctcccacttatgaagggagtccagtcagaaacatgtttgtctacgaggtagtatatctggtatttctgaggaattcggttattatataggattatagaatattcaactttgagtagggtctgtggactccatcaattgcgttctgggggaggtaccatgaaagaacattcctactttagaaatagatattctctgttcaaacctgacaccctgacaggatcctaaaggatggaagctaaagtggtgtggtatataagtcccatgaccttgaacaactggttttatgtgggtgtcaccagtgctttataaaagtaaactgcatcgccccgagttcagtcatactgcaccagctgagcaatgcatggagtggacctgtaggcgacttgcatcgtcttcaacatg

SEQ ID NO: 4 - DNA, 31529 bp, Homo sapiens
gtagattattcaaaaataagtgttggctgggcccagtggctcatgtctgtaaatcccagcactttggaaggccaaggcgggcgaatcatgaggtcaggagtttgtgaccagcctggccaacatagtgaaaccctgtgtctactgaaaatacaaaaattagccagtcatggtggcaagcgcctgtagtcccagctactcaggaatctgaggtgggagaatcacttgaatccgggaggcagaggttgcagtgagccaagatggtgccactgcactctagcctgggctgcagagcgagacaccgtctcaaaaaaaaaaaaaaattgttacttggctttttctatcagtacctatatcccctctaagtaacatatcagagtcccttctttcagaattgactgtctgatcaccaagataatgaacaaagctttgaagctaattctggttattaagcagagaaaagagtttctttcttcacttctctattaacatttcaaatgggatgatttttttttttcggggataagggtttgtcctgaatattgtagggtgtttagcagcatccctggccactatccaccagatcccatagcaccttcacagttgtgacaaccaaaaatgtctctagacgttgccagatgtcccctgaagggcaaaattgccgctagctgagaggcaataaaatggagcaaagctaagaaaaatgcaaactggaggttaaacccatgatataaaatgctgtatatgagatgagtgtgtttgtcccctggaattcagggtgatgaaacgagtcaatccccaaaatcggacatggtggaagattgaaaaagaagctgtggtttgggaaaaccacctaggaccgggagtcaggagaatatgaatgagttcaagttcttccactttctagctattcttgaatcaatgacttccccttgccagagcttcttaataaaatgaaaggatttaaatatgctctactagaatatactgcagcccattctaggtcaaaggctatggtaagtttgtgactcttcataaatctatacttggttctttgggtactgaactctctctagaactagactctcaagaaggctgagtgtgttttcttgtcagagcccaactaacaggttttctgtctttcttttttccttgtctcacaaggttcatttctatatccatcctcactacttaaagagaaatggaggtagaaagtgtgatctggcaaagtcagctcttttgccctaaaatgatagtcatttttccccttggaaaagaggccttaggctaaacagtttctctggagtaagcactatttttttgagctcagaaatataacctaaacgggcaaggaatcaagagtaaacttccatgggaattgtagagcccaggtctctacatacgcctctcttagaaaacggacgtcagaggatattcactgtttcctccataagtaggctgccattttcccattgcatcacagctttaagatgagagattactttgactcccaagctttgttttccagttcagcttcatgcataccagctaattaggcaacatgacaacagggtggagcgatacttgtattctggatgtttccacacccacagtggatttggcctggtgccgctgccagccaccctcagagcagtggcatcttaaaatgcaaccacacccagacaccaacaaattgtattttttaaagattcatgttaatgaaaaattgtatattattgattaatctgtaggcaaaaaagaaaagtgtcagctctttcttcccttttctcatcctctaccttttttgaaatttacagatacaatgagctagccatgtaggtgagtatacgtggtgaaggcaggagttgaggcggcttgacccacacaatgccaatttggactaacccaggcttttggcatatcaggaacacgggctagcttttgcttggcgccttagagcttgtggagtacgttcatttacagtttaattatattttcatgacaaaactgatatataggtggtattatttatattttataaataagaaaaatgggctcagatataaataaacgtgtcaaaaatcacacacaagttgaggggcccagttttgaccccaaatctcattcttttgtcactcttttcatagctattttctatgtaacttaatggtggaatgttctggtctaagatgtcatcaattaattctgtatatattgagtgctattatgttccaggtactgctacctgcagaggcaatatttttattacattggctgatatactcgacaagtacggtttaattggtctgccttctttttgtcttcttgtttcatttcccagtgtgcctcacctcatgagtaatttaggaatgtaatctgtgctcacagtcttgcttcagctaaatgtgagggctggaattaaaacacagacttcctaactccataacaaatatcattttgaatacaatcatgaccttgctagaaagttagagaaataagagggattgataacagcatgaaaggacattttttttttcaataagacatttcttctctttaaagaagagaagatgtttgaacatttaagagctttaaatgagctgtttggttttctgaactatttgaaaagttgtcattttttcatgactcaagtgacctgtcaatccttccagccacgtgtggctattgttcattgaaggtgaaccaattccttccaaagacgtcaccatttgtaatacaataaactgttcaaaggaaggggcactaacaagtaaatgatttctctaccctggaggttactaatttgtacccttaaaattaggcacgttttttggcacttcccacaaaatattcagcacttcaagaaacttagaggtgaattgccccactatgatctcttgtttgatgtaatcaacaggctaacaattaaaagacttctaatgatagctaacattgagcattccccatgtgccaagtatcatctaaaggctttacaacatgaatgaattaacttaatccttgtgacaatcatgattaccacctctcagtttagtggtgaagaaattgagatagagggtttaggcaaattgcccatcctcatacagcaaagaaggcagaatttgacccctgtagcattgctccaaagccaaacactgaatttctgaattatgcttccttctcaagaaggcttggctatgtatagcttgactgttaccaataatagatgcttcagctctgtaatgtaatcatcccgatctgctcaatggtcttatcaaggggatgatactcatggtgatttgcctgaggtcaaagtgttagcttgtcacacatttccaaaatgtcactggtctctgtatggtgcagactggagaagtgatctctgttgacgcactgtctgggtcagtctggagaagacagtccaggaaaatcccgaaatacagtcatacctcagaatagaccaggattgatttccaggacccccgcacataccaaaatctgcaggttcttaagtcctgtagctaccctactcaatttgatgtgataagtgagtccttggtataggcggttttgcatccttcaaatactgtattttcggatatgcctttcggtgaaaaaactctgtgtataaatggacctgcgcagttcaaacctaggttgttcacaagtcaactgtactttactttggaattacggtgtgggtgttttaaaaggataacgatcacgcttcctctccatcaaaataaatcatgacgtttgtagttgaatcatatcagggaaaggtgtcattgagggtgtaggctgcttatttttaaacttgaaataactaggaaatgcactctatttgtcttagccctcctatcttcgtctgtaaattaaaggaaggaggtgtgctagatgaatttcagtattctatccagcccaaaagatatatattcacttagttaaaaaaaattcatttttgttcttaaaaataatacctaatttgtgcagaaaatttatcaggcaagaaaagtaggaagagaaacattaaaaagtcataagtaattccacccatcagagataacaaatgttaagatttttttgaaaacccatttctgtctcccttaacacagacatatataatgctttaaaaatagtatgaaatgaatatgatttttttttctaaaattaggtttactgagatacattttataaaagcaaaattcacagtttgggtgtgcagttacatgacttttggtagacaaaatcacttatgcaaccaccagcagaatgaagatatagagtattttcgtcacccccaaatctcccatgtgtacctatgtagtcagtcccctcccctgatctccagcaaatggcaatcactgttctgatttctgcccctaaaatttgtccatttacaaatgtcctataaatggaatcctaaagtatgtaacattttgtttcaggcttctttcactaagaataattcttttgaagttcatccatgttgtagcaggtatagtatttcttttttaaaattcttgagtagaaattcatcatgtagatgtgccagttggttgacctattcatcagttgttgtacatttggcttgtttacagtgtttagtgattctgaatcaggtcattataaatattctcttatgggttttgtatagacatatgttttaatttctcttgaataaataagtagtgttagattgctaggtcatatagtaagtgtaggtaggattttaaaatcctcttttgtaaaaacttgttttaaaaaatattttgtttcataatttaaaaaaatatttgttttaaaaaagatttacaggaatttggaagatggtacaacaatgttctgtgtacactttacccagtttcctccaatagttaagtctttttttctttagattttctttgtttctaattcatttgactcacaaaataaatgggtgctaactacagcagttatagtgatattattcacactacacagaagaaaccttgggtcattaattgataacatcaaaacctcattcacctacattgaacttccagaatcttctgtaccaactgcctaaaatactaaaaatgtagaatactacaaattaaagatgaataccttggtaggcatgaatgaccgaagtaaaaccaagatttatagagagtcaattaattaaaaatcttccacaaagagaagcccacagccacatggcttaactgacaaattctattaaatatttagtgaagaattaatgtcaactcgtcacaaacccttccaaaaaatagaggaatactattgggaacacctaccaattgattctgtgaggctagtattaccctaatactaaagccacacaaaagcatcacaagtagacaagtatgaataaatatccctgataaacacacacccagaaatgcttaaaaatgctagtgaaatgagtctagcaacatataaaaatgactgtacaccatgaccaaaaaggattattttatgaatgcaatgttgacttagcctccaaaaaccaatcaatgcaatacacataactaataggataaagaacaacaacaaaaacatcaaaaacaacaaaaagatctcttcaatagacacaaaagagtgtttgaccaatccaacacccatttgtgattaattaatctaaaaagcttctgcacagcaaaagaaactatcatcagtgtgaacaggcaacctacagaatatgagaaaatttttccaatctatccatctgacaaaggtataatatccagaatctacaaggaacttaaacaaatttacaaggaaaaaaaaacaaacaactccatcaaaaagtgggtgaaggatatgaacagacacttctcaaaagaagacatttatgtgacaagcaaacatgaaaaaaagctcatcatcaccggtcattagagaaattcaaatcaaaaccacaatgagatgccatctcatgccagttagaatggcgatcattaaaaagtcaggaaacaacagatgctggtgaggatgtagagaaacaggaatgcttttacactgttggtgggagtgtaaattagttcaaccattgtggaagacagtgtggtgattcctcaaggatctagaaccagaaatatcatttgacgcagcaaccccattactgggtatataccaaaggattataaatcattctactataaagacacatgcacatgtatgtttattgcagtgctattcacaataggaaagacttggaaccaacccaaatgcccatcaatgacagattggataaagaaaatgtggcacatatacaccgtggaatactatgcagccacaagaaaggatgagttcatgtcctttgcaggtacatggatgaagctggaaaccattattctcagtaaactaacacaggaacagaaaaccaaacactgcattttccacaatggttgaactaaagtaccctcccaccaacagtgggtaaagaatatgaacaaacacttctcaaaataagacatttatgtggccaacaaacatatgaaaaaaagttcatcattactggtcattagagaaatgcaaatcaaaaccacaatgagatactatctcgtgccagttagaatggcaatcattaaaaagtcaggaaacaacagatgctggagaggatctggagaaataggatgtggcaaatatacaccatggaatgctatgcagccataaaagaggatgagttcatgtccttcccagggacatggatgaagctggaaaccatcattctcagcaaactagcacaggaacagaaaaccaaacactgcatgttctcacttataagtgggagttgaacaatgagaacacaggaacagaaaaccaaacatcacacgttctcactcataagtgggagctgaacaatgagaacgcatgaactcagggaggggaacaacacacattggggcttgtcaggttgggggtgttgggggcaaggggagggagagcattagtacaaatacctactgcatgcggggcttaaaacctagatgacgggttgatgggtgcagcaaaccaccatggcacacgtatacctatgtaacaaacccgcacgttctgcacatgtatcacagaacttaaagtttaataaaattttttttaaaaatacaaaaattagctgggcgtggtggtgcacccctgtgatcccagctactcaggaggctgagacacgagaatcacttgaacctgggaggcagaggttgcagtgagccaagaacatgccactgcactccagcttgggcaacagaatgagaccctgtctcaaaaaaaaattgtgtagtttaaaatccacttattctatcatagaaccagatagtagcagctcatttaaaggatctgtaaatataagctgtaatttttctgccatctccagatgaacacttccttgatttccagtcagattttactgttagagagctgtccctttcaatggagtcatacctgtctctaatttcaatcaggttctacctgtgcattcttgggttatatgaagtagaactaactgttgttgttgttgttttctgcctgatcctaaagaatttcaagcctttcctaagtgttcaaagtgccaatgggtaacagttctggattaaagttgattataaacagattctatgaattctctatatattatgtgaatactttattttcatcaaggaaaacattctctcatccaatttttgtaattcgtggctatctctcatttattaggcacaaaccacttagaattggaatgtcctctccgtgaaatgattctttgtcgtcacgaaatgattctttatccctggtgatattctttgctctggaatctactttgtctgatattaacataggcactcaagcttttttttttatttgtgtaggtatcatatattttatcccattcttttacttttgaactatttttgtatttatatttaaagtatgtctctttaggcagcaaatagtttgggcttcctttttaaaaaatctaatctgtcaatctctatcttttaatttacatatataacctatatctatgtataatacatttacataaagtatattcttacatttaatatatacataatttacataaaacagatttacatatttcataatccgcatggtcagatttgcatctaccatcttattatctttaccaattgttttctatttttatcacctgctttctatttttactaccttttttttctgagacaggatctcgctcttttgcccaagctgtagtgtagtggcatgatcctggctcactgtagcctcgacctctctaggctcaggtgatcctcccacctcagcctcccaagttactggggctgtaggcatgcaccaccattcctggctaatttttctatcttttgtacagatgggatttcaccatgatgcccaggctgctaccacctgtgttttaattccccttttcttcttttctgctttctcttggatgaatcaagtattatgatatcattgtatcttcttttgttaatttactaactataaatctttggttgttgttgttattgtttaaacagttggttattttcaaaagagatttaaataataaaaaaatatatttatccatgtagctactctttttagtgatttcctttcctttgcatagattcatatttctatctaatatattttgtcttctgcctgcctgaaaaatcttcctttaacatttcttctagtgcaggtctgcagtaagtgctgaattcttttaactcttttatgtaaaatcgtctttatttcaccctcattttcaaaagttgatttcatgggacatagaattctagctggtagttttttccttcagtcacttaaagatatagtttcatttcctcttcacctaacattgtttattttttttaatctggtattatcttattctttactcctttgtatgtaatgggatttctccctcacctctctcacccatctaccagctgattttaagacttcttgtctcaatatttttgaacaatttgataatggtgtgccttgatatagtttacacacacacacacacacacacacacacacacacacacacattgtgcttaggattcttttaaattcttggacctcttagtttatagtttcttggacctcttagtttatagtttcttggacctcttagtttatagtttgaaacaaattttggctgttattacttctaatattttttttttacttcccaagacccactttcagggactataattatattagtctgcttacaattatcccacagcttgctgttggtcttatctttttttttctgttttattttggacagttactattgtacaccttcaagtttattaatcttttcttcaacaaggtcaaattattgttaattacatttagtatatttttaatctcagattcagattatgttttgtttttattccttgtctctgctttacatttttgaatttatgaaacagttataataactgttttaatgttcttaagtataattctaacatccgtaacagttttgagtcatttatcctacttgtgaatcatatgtccttgcttcttggtatgcctagtaattttagactgagtgccagacattgctaattttacattattgagtagtgaatgctttttgtatttctgtaaatataaagttttgccctgggacacagataagttacttggaaaccatataatctttttgggtcttgctgttaaatgttttaaagtagaacctatggctaattatcccctactactgagccgcagcctggtcttctgggaatcagagctgtgtgcctgtgcctgagcttcttctccgtgctctatggcttagacattttctcaggccagtaaactggagcaattttagggtttagcttgtttgtttactatctcgtgtatgacttttcttcatttcaaaagactgatgtccacaaccttgaagatcattgtttaatatattgtgtatttgtatttgttgttgttgtttgtttgctgcaggcaggagtgtaaccccagtccctgttactttattttggctagaagaagaagacatccagacagttttttcctattgattttgcctcttattatgggattattttcctgtttctttgtatgtctggtattttttaattttctgctagacattgtgatttactttgttaggtgatggatattttttgtattcctgtaaatatctttgaactttgtcctgggacacaattaagttacttggaaatagtttgcccattttaagcttgtttttaagctttgttaggtggaaccagaataggctttgaggttaattttgccccactactggggcaacacatctctctaatctgtaaatgaatggcaaggtttttctttctgacttacaggaacacaagttatctcctcccatatgtaagttctggaaatcattccttctaatccttagagtgattctttctgcagcctggagtagttttctcatgtacatttacctgtcagtactcaactgaagattctaaataaactatgtgccacaaatctccaaatgtttctctttgaatctttctcttcttgttaacatgccacggaaattttacctcctttggtctcacagaccctcaggtttgtatgatcatctcagggaagcttctattagctacctgtgttctccctctccgtgctacagcctggcaactttctacaggctgtgaactgtggtaatgtagggataacatggtttgtttccccctcccaagaatcagtgcactgtattatatcatgtccagtgtcaaaaacaattgcttttgcttttatttttttctcattgtttcaggtgaaagaataaaatagtttctgttattccatttatgccagaagcagaaatcatatttataatttaaaaataaactgatgtattccttaacccacacctcaatttgtgtttctattttcaggatgtaaagccggcatattaatattctcatttccccaattaaagccttcacattatttgattcctaataaaattataaaagctatgtcttcaagcagctttaacattgttaagaaaggtgaaggacataaggaaaagacagaatcttgatttaatagacttctatctgattctgattatcaccacttattatttgtgtctatggggaagctcctatatctctctaagtttcagtaaattgagaatgataacaatgatacatgtccagcctattctacaggctgatcatgaggacaaatgagaatgcatacagaaaaacacggataatttgtaagcattttacaagtgttagtatttaccttcatcaccaagactagtttaagttcattttttgctttcaatccattgatgtctttcaattctctttatttttctaaagatatacctctcctttggactcattccatattttccttttctttcatatcctagtgtcactgagttggtgtttaaaactctgttgaatatgaaaaaaatatatcagaacatgagtcacgaggcctgagttcctgtctcagctcttccatttatggctaatagatttgagacaaattagtcaatcatgcagatctttaaacttctcatccatgaaatggaaataacaccttctctctccttaatttaaaggtgaggatcagctatataaaataaacagaatgtaaatatttaaatattaatacacacatattgtgtgcaaactggtattatacaagttattctataactatgcactattaggcaataaattatacataaaattgctctataataatataacaaacaaatgcctttagtaatgctaataactgtaagaagtacttaaataattttctatacagtttttaagtacttttcccagcagcataacaagtattttttatctagaagagacagaaaaaatagtccctgtactgtagacaaaagacagttattactttttccagggttgtttatgtggatggcaaataaatgtccactatgtatagtttcttgctcagaaatatgaaaattaaaatttgacctctcagaggtcagagattctatctgttttttttagaagtttaacattgggctctaacacagtgctaggcaaatgcaggcatttagttaatatttgtcatattcacctggagagtattatggagctcatctggaataccataataatttcttaaccttgggtttagatttttaaatttttaaatcacattttagtgagttggtcccacgcctgagaattccctgattttagagttattactcattcgcttcttcctttttttttctgtctattgaatcaaggttgtggtgtgacacaatggttagtgctgtttaacttggtacattcttaatttttaaaccttacaacatggatttgagtctcagcttgaccgcttacatgccatgtggcctgctgtgagtcactgagcctctctacgtttttgttaccttccttttaaaccagccttctttcttaaagcaactgagatgaaataaaacagtaattgtggaaatttgttgtagattataaagcggaattcatctttttgggtttttttactcattcatttattgatttcactaaaaatatcatttatctaattggagagatagacaagtcaactgacaatttcaagactgtgtcatgagtgcaaggtgtggaagtagtaatggcaccgggtactgaggggcaaagagtaagaacactctattaagggtcaggaagggagcctctgggaacacacagtctaagcggaatcctacaagcaaagatatagaagtaaaggaaaagatcatcctacaaaacaaagaagcaatatgtgaatgctttagtaatgggaaacgttatcaagtaaacatttttggtgaacggtatttgggttaaagcaagagaagtctggaagtctgtctagctctgttacacacattgctggaggacatcaaaatgtgtgcattaaagcccttgagagtacaaaagataaaatctatacaaaggagccaaaagactgataacaaatgcaatatgtgaactccaacctgaaagtttgttaaccagttggctttctactttatctaatggcaaagattatttcttcgaccaaactttaatcaaattcctctgaaccctctttttgactgggccttaaccttggcctatgagaactgcaaattcttagcccaaatgatgatgtctacctccctttgtactaagaggcttgaacaaatgttaccacagtttctaatacctcaagaacatgttcctgatgacccagcccctgcttaagttcctatctgaaaagctcaatgctacctaaataatttactatttacctgactgtagacccatgacctcccatttcttagagcatttactttagaaaacttgcatctataaattagcaaacacaaatggcctaaccacaatgaccaaccttccctgacatctcctttagtacttttcctttagcacatgatatggtttgggtctgtgtcaccaccacatctcatgtagctccgataattcccatgtattgtgggagggacctggtgggagatgactgaatcatgggggcgagtctttccagtgctgttcttgtgatagtgaataagtctcacaggatctgatggttatagaagtaggagtttccctgcacaagctctctttgcctgctgccatccatgtaagatgtgagttgctccttcttgccttccaccatgattgtgaagcctccccaggcatgtggaactgtaaatccaataaacatctttcttttgtaaatgcccagtcttgggtatgtctttatcagcagcatgaaaacggactaatacagtaaattggtaccaggagtggggtgtttctgaaaagatacccaaaaccgtggaagcagttttggaacttggtaacagacagaggttggaacagtttggatggtccagaagaagacaagaaaatgtgggaaagtttggaattccctagagacttgttaaatggccttgacaaaaatgctaatagtgatgtgaatgataaggttcaggctgaggtggtctcagatggagatgaggaacttgttgggaactggggcaaagatgactcttcttatgttttaacaaagagactggtggcattttgcccctgccctagagatttgtggaactttaaacttcagagagatgatttagggtatctggcagaagaaatttctaagcagcagagcattcaagagataacttgggtgctcttaaaggcattcagctttttaagggaagcagaacataaaagtttggaaaatttgcagcctgacagtgtgataaaaaaaaagaaaatccattttctgaggagaaattcaagccagttgcataaatttgcataagtagcaaggagcctgatgttaatccccaagacaatgggaaaatgtctccaagagatatcagagacctttgtggcagcccctcccattacatgcccagaggtttaggaggaaaaaatggttctctaggccaggcttagggtccctctgctgtgtgcagccctgcgttgaagacactccagtggctgaagggggccatggcttgggctatggcttccgagggtgcaagcctgaagccttgacagcttccacatggtgttgagcctgtgagtgcacagaagtcaagaactgaggtttgaaaacctcagcctagatctcagatatatggaaatgcctggatgtccaggcagaagttttctgtaggggtgaggtcctcatggagaacctctgctagggcagtacagaagagaaatgtggagtaggagcccccacacagagtccctactgggcaccacttggtggagctgttagaagagagccttcatcctccagaacccagaatagcagatccacaggtagcttgcaccatatgcctgagaaagccacaaacactcagtgccagcctgtgaaggcagctgggagggaggcggtaccctgcaaagccacagaggtggagttgcccaagaccatgggaacccacctcttgcatcagcatgacctggatatgagacatagattcaaaatatatcattttggaactttaagatttgactgccttcctgaattttcgacttgcatggcatctgtagcccctttgttttggccaatatctcccatttggaacagctgtattgacccaatggctgtacccccattgtatctagaaagtaactaacttgcttttgattttacaggatcataggtggaagagacttgccttgtctcagagtgtggacttttaagttaatgctgaaatgagttgagactttgaggcactgttgggaaggcatgatcaggtttgaaatgtgaagatatgagatttgggaggagccaggggtagaatgatgtagtttggctctgtgttcccacacaaatctcatcttgtagctcccataattctcatgtgttgtgggagagacctagtgggagatgactgaatcatggaggtgggtcttttctgtgctgttctcatgataatgaatgagtctcttgagatctgattgttttaaaaatgggagtttctctgcacaatctctctttgactgctacaaagatgtgacttgctcctccttgccttccaccatgattgtgaggcctccccagccacgtggaactatgaatccaatacacctctttcttttgtaaatttcccagtgtcgggtatgtctttatcagcagcatgaaaatgaactaatacagcacacctcagcattaaaaaagcatcctgcctctggttttagcagaattgagcttggtttctactggagtttcttttctctattggaatagcccagataaagtgagtcttgccacttttaataaatattcagctctgtttctctttaacactattctacaggatcataagtttaatgtactggttacttaaagagaaaggagcctacataagactgtattttcaaccgtataactgtgtctatacatttttacacagtttgcatgtgactgggtcaatacatacggtgagtgtattgtttgctatggctgccgccacaaaataccacagactgggtgacttaaacaacataaatttattatattttcacatttctggaggctagaatttcaagatcaaagtgacagcaagtttggtttcctctgaagcttccttcttggtttgtaatgggcaagttcccactgtgtcctcatgtggtttttcctctgtgtgtgcatcactagtgtctcttttgtgtgtccaaatttcctcttctagtaaggacatcaatcagtttggtctaaggcctaaccttataacctcattttaacttaaccaccacttttacttatctccaaatataggcacaccctgaagtagtggggtttaaggcttcaacatataaattttgggcagacagagtttagctcttaacattgagtacctgagacacattagttacagttgtaggcaatgtggttactacggtgtgtagtgtttaagaagctgggctctggagtttgaatgcctttattacattgactgtgatcatttgcaagatcactgcttacaagctgtatgaccttggaaaagttatagaatctcaccaaacctcagttgctttacctgtaagaataaaatgatacttaatctttatatttattgttgtacctaagtgcatgaaatgtgttagcaaattttatttctttacaattttttttttttttgagttggagtctttctctgtcacccaggctggagtgcagtggcgtgatcttggctcactgcaagctccgcctcctgggttcatgccattctcctgcctcagcctcctgagtagctgggactacaggtgcccgccaccacgcctggctaattttattattgttagtagagacgggtttttcttttagtagagacggggtttcaccatgttaggcaggatggtctcgatctcctgacctcgtgatctgcctgcctcggcctcccaaagtgctgggattacaggagtgagccacggtgcccggcctacaaattattttgtaaagataatctttttctaactttgctataaatctaacagtttcattcttccctactgtaaacacacacacacacacacacacatattacacatataaaatttgcaatgcttttttgctaaaattaggtagacttttttttttttttttgagaaagagtcttgctctgtcgcccaggcgggagtgcagtgaagtgatctcagctgactgcaagctccgcctcccaggttcacgccattctcctgcctcagcctcccgtgtagctggggctaaagacacccgccaccgtgcctggctaatttttttgtatttttagtagagacggggtttcaccgtgttagccagggtggtctcaatctcctgacctcgtgatccgcccatctcggcctcccaaagtgctgggattacaggcgtgagccaccacatccggccggtagactcttaggtacttcaggattaatttgttatgatttattgaaagactgtagctcctcaaacctctgttttctattgtcaggataaatcctaggagactttaaaatgcactaaatttttgagtgataaacagtgtggggccatgggtcaggggttcaagtaatatcagctatagggagaagctgattgttgtttctaaaataaaatataacccatatgaaaatattaatacaattaaaaattagacattgaaattcctgtcttcttaatcctaatctaataaaagcagtaattatgtctgtaaatagcagggtctatatactcagtaaacaaacaatgcccatcccattctattattagataaaatgagaggcttatctctagtttcttagtctttccagacaaacagaaaggaattcctacagagagagtgaagttagttggtaatgcaacattaccagataagaagatgagtaaaggctgttttccagttagtgtcttttacaaaggagaatataggccgggcgcggtggctcatgcctgtaatcccagcagtttggaaggccaaggcgggtggatcacgagttcaggagatacgagaccatcctggctaacacggtgaaaccctgtctctcctaaaaatacaaaaaaaaaaaaaaaaaaaaaaaaattagccaggcacggtggtgggcgcctgtagtcccagctattccgggaggctgaggcaggagaatggcatgaacgctggaggcggagcttgcagtaagccgaaattgcaccactacactccagcctgggggacagagtgagattccgcctcaaaacaaacaaacaaacaggagaatatagacatgttagattgatgggggaaaatattagaattatagaaaagaaaataaaaaaataaaaaagaaattatttcatcttttaagatatattagaatatataaattttactcatggtcacaacatacctctttttgcccaagtgattgtgtatgtgtgtctgtgtgtgtgtgcatgcatagggctgtatctagtttaaagcactgtaacgtctggattctctttttttttttgagatggagtctcgctgtctcccaggctggagtgaagtggcgcgatctcggctcactgcaagctccgcctcccgggttcacgccattctcctgcctcagcctccggagtagctgggactacaggcgccctctgccacgcccggctaatttttatgttttcggatttttagtggagacggggtttcaccatgttagccaggatggtctcgatctggattctctttgaacggtataaaagtctgtgagatggccgtggggaaactcagagaaattaaaggtataaaatgtaagaaagtgtcaaggctgagatgaccatcccagctagttcctactgcaggtttcttttatgacaccaattttggctggtaaagaaaagataataacacctaattggctgcctcacaacactgcataaacaaatcaaaagagctaatgaatggcaggttgttttgaaagctgtggaaggtgatctaatcataaaagactatagttatttattaactattatcgtcatcaaggctccctctgtgtagaaaaacagaagactttaggaatgatatggtaactttatattttggaaataacctttgtttgtagtaacattactgatctacaacaatctttataattttctcatttcctccccctgtatcagtatccttcagattctgccaacctcataaatgcgtaaggttgtaaaattcatagtttttattatctaagcccactcatttctaatgagaggaaaaaagtttctacatgtttgatgaataattgaaagagttaaaaacaaggatgatattttgagttagaagaataccttgataacctaactctgacccaaacctgagttctccaaaccttcaatcccattcatttaaatgtaatattcatggtgattttcaggaatgaggagtaattaacagagcatgtgatgaatcacctctggctttacaacattcctaaaatgaaatggagaagaagccctaatgaaatgagccagctgatgagtacttccaattttgggggtgctcactgaatcttctttttactactttctccatttggaaaagatatcagtttcaaactttttattcatcatttcagaaaaggctggcatctctgaggagctcttttccagtaagaaggaaaccaatgtcttcaatgtttcctcatagtagagattcctaagacaggcaatcatcttttggttcttctgttccaccctagcaccacttccactcctgggaatattctccatttgggctaggctaggggtcactagagcataatacacagtcattgattccattttaaggcatcaaactcttttaccgtcaataacagaatatatggttgggacacatttcgtatggtctgacagggcagatgaaagataaaaaattagtattgatatgtgaatatttagaagaggcttttagagaaagagacaagaaatcaatgctgattgaaacagagcaatggttttcgttaagttttaacaggtgatgtttcgatttgtttaggcattatggtttaaaatacaattaaaagggaaaaagagaacatatatcgttatagtttaatttcacctcaaattgtgttggggtagtttttctatgctgagtaaatataaatctattggtgaaatccacagtcaacagatgtttacttttcttggtatcttagaaattatctagtacaaccacgttattataattatgtaaaaatgcaagaccagctatcattaactatcaagcttatcacataaacttgataattgttctaagcacctctctctctcacacacacacaaacacacacgcattatatatatgatttatctctaaacctcttagcactagtacaatgtacgtgttattatttttattttacatattaggaaactgaggcttagagaaaataagtgacttgtccaaggcaacatggctaacaattcaagatttggacccatttctagcttgcttcaaagctgggttcatttctatccctacatagttgaaaagactacatagaatagaaacagatttctgaaaccctgtctcattctctttccattaagctatgatggactttgattttctaacctgtaacaataatggaatttgaacttgctttccgtggtcactaattttactattccattggaaaggagccaagctacaacaatagtgcagaccttgaaaccaatgattttatatatatatatatatgtgtgtgtatatatatgtatgtgtgtgtgtgtgtgtgtgtgtgtgtgtgtgtgtatatcttttttttttttgatggagttttgctctgtcgctgaggctggagtgcagtggcgccatctcaactcacacaccctccgcctcccgggttcaagcaattcccctgcctcagcctccagagtagctgggactacaggtgcgtaccaccaagcccatctaatttttgtattttaatagagatggggtttcaccatcttggccaggatggtctcgatctcctgacctcatgatccgccagccttggcctcccaaagtgctgggattacaggcgtgatacactgtgcctggcccagtgattatatttttataggaaataaattatttcatgagagaaaaaagaaaggacgtattatctcactgtgataaatctcataatattggcatactccctaagttgggcaactttgatcagttttctggaaccatcataaaaaggcctttaattggataaagtctcgaacttttcttctgtggatgtgaatatcttgagaagtactggaatgaaacaatttggctctggtttcagtcatcttcacatgtttcttcaggtgctttcttaagctattagggcaaaataagctggtagaaaaaaggtgttacttgtaggatgaaagaagcaactaaagagaagaaactttctctagaaaaatgttacatttacacatttataattgttaacaacttttttcttcttttctttttgggagactaaataatgggatgatacaggaagaaatatgatttagagaaaactgattgagccaaatttttcaaacggtaatatgggtaatcataggctatggaaagttaaactgagtctccaatgtttatgattatgatgtttggctaggatataaaataaggcttcagaatataaaatatcctgcagattgagaagttttaaaagtagttacatacaagttttatttttccaacgttcctgccttttctaatgtctaaggatgaagcagtgtacagagaggaaagaatgttatatgtgtggtctgcttgcaagtgctggatgcctcctctgcctgctccaactaaactgtctgcttatgagaaagtcattgctcctctgagttcctgcctccctatatattcaacattagaattttgccaggttatcttttagggtcgttcaactctaacatatatatgagtctgtgacactgaactcagacacttcagaacaggaaccctggatttctctggatgtggaaatctttaaatctatacctgactggtaggacaaccaatctgtcttcctttcctctcctctcccacttatgaagggagtccagtcagaaacatgtttgtctacgaggtagtatatctggtatttctgaggaattcggttattatataggattatagaatattcaactttgagtagggtctgtggactccatcaattgcgttctgggggaggtaccatgaaagaacattcctactttagaaatagatattctctgttcaaacctgacaccctgacaggatcctaaaggatggaagctaaagtggtgtggtatataagtcccatgaccttgaacaactggttttatgtgggtgtcaccagtgctttataaaagtaaactgcatcgccccgagttcagtcatactgcaccagctgagcaatgcatggagtggacctgtaggcgacttgcatcgtcttcaacatgaagatagccacagtgtcagtgcttctgcccttggctctttgcctcatacaagggtgagcaatttgtgtgtaatctaagcctcttgccacacatctcaaagccctggggaagggacttttctccccctactcctgccaaaaaatgtttacgtatgcatgtataatcctgaaatgtcttgccagacacagagttctgaagatttatatatgtggctttataattatgttgttatctttagaaatggcaagtagtttgagataatctgaattttctaaccatgagaaattcttccattaattctttattaattcagaaaactgaatagattcttcttcatcttcctcttgctaaatgtatcaattcataatgcatattcttatatatgtaatagacatttaaaacttaattgtaaataatatatgtattttaatgttataggtataaatgaaatattttttaaaattcaatcatctgttcttattttaaagcagtggtatcttctagcttattttagtcttgtgttttcaaatgctaaatgggttgaaagcatttctcccaaagagtgagggacttttgtgggagctggaaggtagatttttatatttgctatttaaccctaagagaacacaaattgcctagacttacgttgctctgaaacattaaaatttttaaaaagagaaactcatagatagtctgaaatcatatacaaataatgcccattttcctaaaataggtgtgtaggtacaaaagagcctatgttaatgagagagtataatagcctgtgaccttaatcttaccttctgtcagatctctaacttttgcagctgtaataaggaggtagagctgccagaattcagaaaattaaattttatgtatatatcctataaatgtaatgctatattatagtttcaactataatatagtttcaaaattataattggtatagtattttgtgtgtatgttatattgtaattttaaaagggaggctggcatttattactccgaaaggaccttttgaagataagctattttgatggggttagaattcacatgagtattattttccaaagtaattgtaactttctgagtgattagacctttgtgagtatatatactaatctcctggtccagtgcccttcttttatttgccatgataaatcttaatgtgttcaaaaaccatttgcggttttacaacatattgtcattaaagcaatcaagatgctgcattaaatggattatttgttttgtttattaaatattttacatttccttaactttggtttctatattttcatcccagatgctgccagtaagaatgaagatcaggttagtcctgctttttctgttcattgaattcattccaagattcccaaagaaaagtggtttgtggccaacaccttcatgttaataatacaaacatattatactggtaggtactaatagcttttgttaaaaacacagaaacagatttttatgaatcaggacatgagcctctctctcacacacacacacacacacacacacactcaacatattaatgatgatggatgtctgcaagaggcaatttatcatgttaaaggagcattttaaaatttcataaacaaaaaataaatcaactggaaaactactattgttccatttaacatctcaacaagccatgatacaccacataggaagctgaaataaattttctagtaatcattatgcaaaagagggtggtctgtatcttgtatatagtcggtgtatatagttcatgtaatgaatatttattgagcatctcttagatgtcagaccctgtttgagatgctggggttatttttaagtaaacaaaatagatgaaattcctgccctgaaaattacgttttagtgtaggaaaacagacaaattaataagtagcttagcaaaacatatggtacgttaaataatgataaatgctaggagaaaaataaatcaggaggcataataggatccctgagaatggaggtggtatccattctcataaggataatcagggaagttctctctggacagacagtattagaataggaatctgaaggtcataagggaatgaaccacgaaggtatttgcagtttacagcaggccagacagaagcacctgtgagcgcagaggccctgacgcaagaaggtgctttaccaactcttaaacctcttttgtttgataaattaaaaaatcattttctactagaagttaaattttttaatctatttgaaagataggatgaccaattactcacacacacacattttatatatttatatacacacacactgctatgcatgctattaattcagcacttaaagcacaatcttaaatgttttgctgaatatgtggcaaaatttgttttatctctggccacgaacattaaaggaataaaaatgtgaaacagctattctttacgttgaaactctgagaaccatggttaaaatactctcttgccgtattttgttgcaatgattttttttaaagaaacttcttacatgagtgttctagagaattttatcatgttctctactatttcatctaagtttactgatagggagaggtccagaaagttcagtagagtagaaaataagctagacatttgggaatcagagagcatgctcacttggaagttttatactgggaaaaaagaaggatattatataaatatcattgtgtataaaaccatcattttctttttcagagtccgtgatgatggttggtgatttatggcagatactgtgaattttacactgctatttttcatagcattaacctctatttcactgaaaaatattttaaaaacaaatttagtgacttcttatatttggtattaaaggcaagaacacaaagaagtaattatttatttggaagcctttctaatcagagtttttggatactgtgtcagaaatgaaaactattgatgtagtcttttttacgatgctgtgattttggttattttccaagtttagtagcttgggttcctttcagcatgataaaagcatgttaaatgtcataaacatgaacctaaacagcagggaaaagcactgcagcaatctgcaaggatcttaagcatgcttaaatttgtcatcacctaagagtcattcttcaggcaaaagcaagctggtcagtgctctaacactgagatagggcaatggcagctcctggagggttttattataatatctggagatggacaatagaagacaacaaattaggcctcagccagtgagggaaacaggaagaacttggccctagagggctccagttttcctgggacatgctgagaaaaagagaataacagcaacagtaacaaaacaaaacaaaaacaggccagggactggaagttaccctgcaagtaaacaggtgctgaaaagtcatctactcaactcaggatgacttttggtctaaatatgcttctgtttcaaccctctctattatggtcattgaaagggtattccattttgctcagagaaaaactcaactgctgctttccatttttatggtcttttcctgtttcccaagagaatacattggataatttcataactcagaaaaatagcattgaatctcatcttaactaattaatcactgtgaacaaatttcttaacctattatttgggcctctgatgctcagtttgtaagaaagggagtctttctaggaattaagttctcatctaacagtgatattggagaagtccaatttagggattattcttattgatgcaactctttaatttttcttataagtagagacaaggtctcattatatttcccaggaggatcttgaacttctggcttcaagcaatcctcctccctttgcatccaaaattgctgagttacagccatgagccactgcacctggcctgatgctactcttttatgataagctttcaaaattatttagagctttattccaggattatcatgagtcgtctatatactttctataagacttacttaataagggatatattttcattacaaagaccccaggctcttctctgattctactatatggattttaggatcaattatattcatcaaggtaagcataacttgcaaagttggcatttgatactgtagaatcagagatcagacaggtctcaagatgcccagttattaatctgtgtccagaaattactactctgctactctgatggagtcactgtgtagctataataatcttagagatatttaagcatgccatccttagctaaatttcccagggctggtctgtgatatcgtttagttattctccttcaccattaatcattttgcaagtgctaccaactgcctaccatttttaagaggctagattaagtacatcattattcatccaggcaagtatacttctgttctacctaatgctgtatctatgtcacctagcaaagtatagtaatccaataataagtgattgctggctcattcaatgatgatcataaagatataggagactactggagttcaagaccagcctgggcgatgtagtgagaccctgcctctccaaaaaaaaaaaaaaaaaaaaaaaaaaagaaagaaagaaagaaagaaaaaacaagcacaaaacaccagatatagatataggagcctaaatagaaacctttaataaattttttggaacaggttgtgtttgtcagagttgaagcaaaagctttgtttatttgtttcctgatacaagcaacttgaagcagattaaaaaaaatcaaaacaaaaaacaacaaaaatccccttagattccaaaggcctattctaaagacctagtgaattttcactgggtacagtggctcatgcttgtaattccagcactttgggaggctgaggcaggtgtatcatttgaggtcaggagttcgagatcagcctgcccaacatggtgaaatcccatctctgctaaaaatacaaaaattacctagatgtggtagcatgcacctgtaatcccagctactcagggggctcaggcaggacaacctcttgaacctgggaggtagaggttgcagtgagccaagatcatgccactgcactccagcctgggcgatagagcaagactccatctaaagataaataaatacctagtgaattttctaaattaaaagggaatataacattttaaaatcatactttactaggtagcagaaatgcaccatattttaagctcctaaaaaagaatgagtgatgtatagcaggactttaataataatatttaatattatttattaatatttaatattgaatgaatcaaagaattaccacttttgaagatcattctactttccttttccccaccaattctattatttaaaatgtttcccctgtcctccttaaactttgtataacggagttattggtatttacaaaaccaaaagcccaaaacaaacatagtaggattttagttccaagggatgtgccaaaaaaaaaattaccagagagagagagaaagagagagaaagagagagaaagccttatgcaattattgagaaatttcctcagtacttccatagattcacaaggtttgattctgatggtagtgatagtagtgctgatgacatttgtggggatggtggtgggggtggaagtatgggtactttctaaaaaatataatacaaatttgaaaatgcaaaatggttaggcctctcccagaacctcggaagggggatgtgacagtgaagggccctgaatcttaagctttattagctttaaggtaagtctgcctgtgctcataagtcaaatgatttatcaagggacatgaagttcttttcagaaaataaaccagtctagaaacttgttttccagataaatataaccaagaaatccttttttcatttaacactgttaaaatttctaggaactgatatgttgcagtacagccatggggaaatgtaggcagacattttataatgttcaatgctcaaacaccttgcctcagtccttcagcaacattttcatattcctttcacagcaattactattggcctactgtgtactaggtattttatatgtgtcatcttagcatattccagtaataatactgacaatcaccagcaacatcaaaacaataataataaaaaaggaggaagaaggaaggaggataatttattaattgaatatctactatgtatcaggcattctttgagggctttacatacttaatcttcaaaaaaccttggaattgtgtgtgtatatatatgcagacatatacatatatctacatatatatatcaaaataaagcttgttttgatgtacttttagctaacacaacttttttggcattatcttaggaaatgtgccatgaatttcaggcatttatgaaaaatggaaaactgttctgtccccaggataagaaattttttcaaagtcttgatggaataatgttcatcaataaatgtgccacgtgcaaaatgatacttgtgagtaaaggtttctttctttctttccaatgtttgagttaacagctagtctctgaactggtaaatgtattctttttctttcaagtgcatttttctaaaccgaaatggttaaataaaagtgctgagcagatcactctgacatttcccatcagataaagttggataattaccacccacctggaaagattttaataatcactcaaacaagattctgaaagcattccagagggcccagtgtcaataccttctgtagcactctatagtcttttgaggatgtagttccactttgaacttcctggttttggaaaaaaatgaatttgacaaagaaagtcagattccttgggcctgttaagacctgattctgttgcagcctagggactgagaagattggcttaagcataatgccagactctcccgtgacagccaaccagcagaaggacttaagcaaggtataccctgattctgtgagctgccagtgtctttagacacctagcattattctaggatgagatgaactaaagggtagattatagtttccaactccttgcagctcagaaagaattagtatcctggaagtttaccaatctctattcaatgcttcttaacttggtttccttctctccattgtcaccccaaaggtaaaagaaggcttgggttatcaattgcaaatcctcaaaccagaatgtcctaatttgtttttgaaaaggaaggttagcctcaatttctgaccactgtcaaaacttcaacagtggtagcaagccaatactgagcttacccacttaggataattatattacaattggctttttaaatgtcaggttggtgtgtgtcacaggatttaatttggggattgacgtagtcaatcatattgggataatgaagctcagatcgtttgatcatgcactgcttctagtgaagagactgtgagactataaagaagccaggacaagatttaatagaggatgggaggcctagggcaggttgaataattctttctttttttgaaagctgaatgtaaaccagcaggtatattatttattataaaattttcttcttgtttcataagatgttttaatatgagactgaaagtatgaatataataaatttactgaaaaataatgtaaaacaaaaatgaaatgccttaagagtctaaagacagtttatcattttgaagttattcaattattatttttatgacttacgtaaattatgttagaataaaatttttttttttacctggaaagaaggaggctgtgcaaagcataaaagctgtcatcagattcctaaagaactggcttatggaaaagaaaattgatttgctctgtgggtccctagatgttggaattagaaccaatataagcaggttcaatgtgaatattctctttcaaaaactgcattcaaaaaatggtgcagatagctttgagagttggtgaccacctggctgttggaagtattccaggaaggtagtctgttatgcagttaaagttagagattcttcctataataccatgttcgagatatttttcaatgttgaagggagatctggggttctgtgtccacttctgaacatgctaccaattttgacatgccaggctaggctgaggagagcagttaaaattaagtttaccatgttagctgttattgttaaaagttgagcaaacaatgtttaaactatgctatttcttgtccttttccagggaaaaagaagcaaaatcacagaagagggccaggcatttagcaagagctcccaaggctactgccccaacagaggtgagactatttggagccaacctgtttacttttgagaggatgtttgactctatttagagctatctgtttattccttaattactaggtcatacctgttttgaagccaacaacttaggagggagggaacatgggttagtccaggggtgggctaaatatctggtacatcttgtccataggcccaacccatgatccgactctaaaaacattttaggcactgacggaaactaagaggtggagtgagccagtgtgatgtctgccttccaaggccatcatttctcattcctcttgtcctgtcttagcccaaagaagagctttcctgggactgtagaactaagttcaaatcttagaattcttattgcttgtgtaacttagaaaatttatgtatctctggatctcaatgttttcatctgtaaaatgagttttagaccgaatagaaactaagttttaaaagccggtctcagggtttaacatgttctagcaggcatttgactcattctgtttcctttttcttttactcttttgctttttactgtatatgttccagtacctggagggtcctcctaattcaactatagttgcttgtttatatctattttgcagacagggactcaggctaatattttgtttaagaaaagtgttctctggtaaaaaaaaaaaaaggtcataaaacattaagaagtaagatagaaggaaggaaagaaactgcaaatttatcctactatataaaccatttcacttgataatttttttcacttagaaacatttttaaaatctttacaaaattataaaaataaaatttaaaaaataaaaatttggggtccagattttagagcgtctcagtggcc

SEQ ID NO: 5 - DNA, 7995 bp, Homo sapiens
  misc_feature (7994)..(7995): n = a, c, g or t/u
gaaatggaaaatcataatagacttacagcataagtaaagacattgaatcagtaattaaatttaaaaaaatgcccaagagcagatgactttattggcaatttccattatatactgaaaggagaaataatatgaattctttacagattcattcaggaagaagagacactttacagctcattatatcagtccagcattattctgatacaaaatgtagaagacatcacaaaagcagaaaactactgacaaatattcctcataaacatagacagaaaaatccttaacaagacactggctggcaaatgaatctgcaatatgaaaaaaataaatcattaccaaataagaattatcccaagaaagcaatgttggtttgggaacagaaaatctattagtctaatacacaatattaacagaataaagggcaaaaccacatgattatcttaatagatgttgaaaaatcacttattaaaatccagcactcattcaagatgaaaactcacacaaaatattatcaatagaagtaagctttttcagtctgataaagaacgtctgtgaaaaacctacagctaacattataattaatggttagagaatgaatgctttgcccttaggattgagaagaaagcaaagggtggtttactgtcaccactcctattcaacattgtactggaggttcttagccagtataaggcaaaattaattaattaattaattaaagacactttttattggaaatgaagcaactgtctttaattccaaaacatgataccatatatgaaaagttctagggatccacaaaaagaaaaattactagaactaataaatgagtttagtaagatttcggtgtgcaagattaataatacaaaattgtatttctatatactagcaatgaacaatttgaaaattaaaatatttcattcacgatagcataaaaaatgtggaaatgaatttaacaaaatgagtacaagacttatacactaaaaactataaaatggtactgagaaaaatgaaagatccaaataactagtgagataaatagtccatgttcatagatgagaaaactcaatattgttgagatggcaattttcccaatattaatcaataaattaaacatattcccaatcaaaatcttgtcagtctttctttttggtagaaatagagcagttgatcctactagttatataaaaaatattgagacatttaacagtaactgaatttaaattttgttataatgcaatagtgatcataacagcgtgccgttatttcaagataggcataaagatcaggggaatagaatagggattacttacttttatggtcagttaatgtttaaccaacatgtcaaggtaatgctgagaaaattgaatataattaattttaaaaaacagttttatcttataccatacacaaaagttaacttaaattttattttggcactaaatgtaaaagctaaatatatgaaacttctcatgtaaaatataggaggaaaaaaattcatgaacttgggtaggtagagtgactttagacataaaactaaaagcgtaatccatgaatacaaaattaataaactgaaatttataaaaattgcaaatttgtgtgcttcaaatgacaacaagaagtcaatgaaaaagcaagttatagactgggagaaaaatagtcacaacctatatagcaggtaaggacttaaatccacaaaacataaaaacctcttacaaccttattataaggagacaattcaattaagtatagatattacaccaaagatatatgaatggctaataagcacacaacaagatgcgcaacaccgttattatttaggcaaatgctaattaaaaccacaatggtgtatcactacacgtacccactagaatgattataatcacaaagacagaaaatgcaaaatgttaagggtgtggagaaactggaacacatatagtcagatcggggtaatatacaaggttacagccatattggaacacagtttgagaacttcttaaaattttaaacagattgaccatgtgatccagaaaattcagtcctaggaattcagtgaagtgaaaatacaggtccatagaaagtcaggcatgtgaatatttatagcaacaatgtttttaatagttctaaactaaaaacaatccaaatgtccaaatgtccactggtgaattgataaacaaaaggaatatatttatacagtgaaatattattcaacaattagaagggatgtgctactgacaattttgcaatatggatgaacttcaaaaacactatgcaaagtgaaagaagctagaattaaacaattacatatttataattccatttatatgaaatgtccaaaaagggcaaatttacagagtcagaaaaatgtttaatgattgcttgaagctggaggtgggggctggatatactgtacacaagcacaggggaatctcgtagggtgatgaaaatattttaagaccagattctggcgatggctgtacaactctttccatttactaaaaatcactgaatcactttcaatgggtgaattttatagtatataactaatacctcaataaagctgtttagaattttatttttcagttcatatgattgtaactgctttggaatgatttgtacattactattatttaatacaaaatttcctagaccttcagaaaaatagattataaagctattgggactgagttattttggggtgggaatatctcagaccaatatatcaattggattaatgtttattgacctgtgtaacaaatgtagttatcttatactttttcatagtcatatatgcaatttggtttttgtaatgtattagcatactgatactgatgaaatgctttcatcgtattaatgtctcaatatttatatggtagtttttaattatcattccatatatttttacttttcatattacttgtctctttggtcagtcctactagaaatcttttatatcaggttgtttgataatatactctgttgtttctgttttctatggtgttaattcctgtttctttatgtttgttcctgttcattttacaatggggttgaggaatactttattctttaaatttaatttttaaaaaatttatgacaaatatttttgaagatattaataattatacatctcaggatttatactttaagtatttctatttatcattattttccaaacacttcttaatttctctacccctaatgttatttagtaatagattatctaattttcaaatatgtggaacttttaaatattttatattagcttttaattttattaaattttggtcagagaatataaatgatgtagtattgattatttgttatttcttaagactttgtgacctcacaaatattctgcttttatgaatgttttatatattctttttttttttttttttttttttttttgagacggagtctcgctgtcgcccaggctggagtgcagtggcgcaatctcggctcactgcaggctccgccccctggggttcacgccattctcctgcctcagcctcccgagtagctgggactacaggcgcccgccacctcgcccggctaattttttgtatttttagtagagacagggtttcaccgtgttagccaggatggtctcgatctcctgacctcgtgatccgcccgcctcggcctcccaaagtgctgggattacaggcgtgagccaccgcgcccggcctatatattcttaaaaaatgtttattttctgtttcttggttgtagttgtctataaaaatctaatggcttaatattcttttttttttacttaatatataaatttctgagaagtgtttgcttaaatctctaactccagttattgatttatttaagtctattacattctgtttgttattgtttgacatatttaaagtctatattattaggtgaatatatgctcatgattgttatactttcttgttcttgttcttgtcattttttacttcatatattgtccttctttgtcacttatgatacatttttggactcaattctaatttttaagtgttaatattgctgcatatgctctcttcctcttcatttttgtcttgtatatttttattatcttactggtttgatatttgcatgccttttcattttaatttttctcaatacatgggttcttttttaaacatcaaattaccaattgtctcttaatttgtaggtttagcacatttatatttattgttatttctgttatagtgagacttggttctttcatttttaaaatattttcggttactatatttagtagtttcttctttctagctttttttgggtggtgatttatttttgttcttattatgactatccctaaacaaatagccatacttttgattatttattctattgctaatatctgtgtctattgtcaatttctttgtcttctctccaaatgtatgttaatccactctccatatattttctgtaaatataattttgttattaacttgattttagcttaaaattcttattgcttttctcttttattctttttttgttaattatagaaattgttcctttcttcagatatttctttctatatttatttccttcttttggagacttctaattctattttaaatgttctttagcttttccttttttagaaaaattgtttcttatttccattgccttctaggagaattccttgatctgatcatctggttcacaagttcattatttatttgtacctatcccatctttattctttatattgctagatttattatcatttgaatattttccatatacaaaatttccactttgttcttcctttataacttctttcttgttattgttttacatttgtttatgcagatatcttctcttatctctccataacataattagacttgataaaatatttttctacccattccaatatttctacttcaattaatacatgttgtttagtatattctattttttacttgtcagatatacagatattcttattgtaatattccataatggctgtatttattttttctgctaaattcagttaggtagtgttcaatgtcaagcttcagtctgtagcccttctggtgagtataaggatagaaaaagagataagacccagtgctggaggttctgaatcccctcttttccactccagcttgctgccacttcaccaggtcaggaatctatctgtaaggtctcaggcattgtcaacattaggaagaggtgttttctgtaggttgtgatccaccacctaggggtttggaggggaaggagatggagtcatatatccttgcgcctgttttcatcatcccccagctggccttttgacttgctctgtcattcccctctctacacctgtgacctagttagtacgttgctgttgattttctggggaagggggatagtagtgattgttttgagttaattactttgatctgtaatttccctatgcttctgtagtatcttcaggaatgatttttgagagaaaaccaagagccatcctgctttttctttatgcttcggtcacatttcccttctttctgtccattccttaaaaaggacagtctagtttccaccacaaatcctttgcaattgccgttctctatgcccagaatgttcttccctcaaatgtttttgccactatatctaaaatagtccccattctggtcactattgtatttttctgtttatttgtgagagcacttactataatttatagttgttgtgcatatctgtctctttctatcagaatgtaagctccttagaatatgtattccatttttttctctctctctcttttttattttatattttctgtggcagcttcacagtccagaagagtgctaaaatgtagttaatatgtaataaattttgtggaataaatgaacaaatgaatgaatgaaataacttatatggccactgcttatcttattttcaatgtatttttaacttctttctaaacatatgaatagtggagtatgttctttagcttttcttttctattttcagttcttttccttctagataagagtttctcaacctgatcttctgtatcctacttttattctttatattatgtgacatactgatacatcataggtctacattctaagtgtttcatatttatcattaaaatgtctttattttgacaatgataacatgaagttgtattttgcaatgtgatattctaaagaaaaattatccagttttattatttggttttaggtaaaagttaaagtcaagttaactctatatcttttttagctaggttatgtcacaataataagttgccaaataatgtttcataagaattcacttgatccttattgcaaatctggaatgtaggtagggtgtgaatttccatttttctgatgaggaaagtgaggtgcaaagaggtgaagtgagtgggtggcaggtctgagacttgaacactagtcttcagttattactttgtacatttgtttttctttaccatcaggtttgttcctaagtgtatgtcacattattagaatagagatttgggctttctcctgccgtacctaaaagatgtttagtttacttggcttgtcagtagagaaaacattgaaaataacaacaacaacaacaataaatggttctgtgtcaggcactgttccaagtgctatacatgaattaacatatgtaaatccctatatagtcctgagagtttgacaaaattataaccattttgcacatgaggacactaaagcatagaggtgttaaataacttgtccaagtcccatagcttaagtggcagacttagaaatatgaaacaagcaatcctgctcttaattactattctaatctataataatagaaactttgctgtagtttatagtttacttagagctttcatatccgtaatctactgggcctcactactactctgtggagaaggcaggcaggattggacagacgaacaacatgccaagtttcagagtggtgacgtggcctacttcacccagacagcaagtggctgaagtaatactgaaattctttcttttgaaatctaattttatctgttcttggatcatgttttattgcccataaatttattagctcaatgtagccttcatgatatgtttttcttctccagcaatttacattattgtgggcttaaaatgttaatctatttatacatctaaggactattttgtttcttacattttgacgttccttgatcatgtcttttgcagctgaattgtgatgattttaaaaaaggagaaagagatggggattttatctgtcctgattattatgaagctgtttgtggcacagatgggaaaacatatgacaacagatgtgcactgtgtgctgagaatgcgtgagtattctctgaagtaggctttctccctaaaacgtgttctctctataattacatgacacaattttccctacagtctttaagctaacgattcattatgtgggagttagccattcctaaattattagtaccacttcttttactactcttaagacaaataaaggtgtatagcataacactttatattttacaaagcacttttatgtatgatttatcagatggttatcactttatgaagtagacagaatagggagattatagattatatgttcataatctataaaatgttatagattttatatttaaaatgtataataaaatatagctttaatataataaaacagatttaatattttaataaaatatagacttatgattttataatctataagttatagattatatgattatcatatataacagattatataatatatttacacacatacatacatatatacatatatatacatacacacatctatcttctatctatccatcatctattatctatttgtttatctatgtgtataccctttttcacagagaattatttcatcatcaaattatgtttattaagtgcttaataatttgtaatattagagtagataattgagactttcaatattaaatttcttcccatctctcaacatttaccaaatagctaaggcatcttactgtcctatgnn

SEQ ID NO: 6 - DNA, 959 bp, Homo sapiens
  misc_feature (1)..(2): n = a, c, g or t/u
nngggccctatttattggcggaattcccattggatttggaaggcttgatgaaaacatttccggtttcccagctggtggggccccaaaattgaatttttttaggcccccgattccttaaaaattttttccctttaaaaaggttttcaaagggtttaaatttttttttccaaggttcccggacggcttactttttccaattgggaattttttccatgggtttggtttcaaaaaactttagggttcggcttgccaattttttttccagctttaggccttttatgaaaattaactagtttcataccccaatagagagtgatagtggatgtaataatccatttgctttggttatttaacccaaagttctgggtgctgagtgtatctattatccagttccagcttttacttttccccatcttctgatgtggatccctcctctaacaaaggttaaaaaaagatttctataaataacatttacaagtctgaatctttaccatctcttccatgttaactacatttctttgacttgaagttataaacagtgacttttattagaaaaagtaataaaataatactatgtggcagctgttttcgggagtaaaggagaatgacaatgcaatgtagagcagttaggtttgaatggtgggaagttctgtgatattaaactgctgtgtctactaacttttgattctaggaaaaccgggtcccaaattggtgtaaaaagtgaaggggaatgtaagagcagtaatccagagcaggtgaggtcaattgtcagcctgatgggaaatactgggaggctaacttcaaatagtaagtaggtgctgtcctcttccttcttaggtgggagccttggaagaattaattcttgctttatgtgaaatggaatacccagtactgcccaggatatgaaacccatattatagtggcaatccaaaggggcaaaaagacaacctaaaactcnn

SEQ ID NO: 7 - DNA, 752 bp, Homo sapiens
  misc_feature (729)..(734): n = a, c, g or t/u
ttaaacaagtaaaaaagttttatatttacccacatgaattatagagtacttactgagctacatctacacagctggtactgctctaagtggcccatagcaatgtcagagggactgagttcaataaaatttctgaaaacagtgttgtcagcattacaatcttggtaagtttcaagttcttttccctgttcttcaggatgtatgcagtgcttttcggccctttgttagagatggaagacttggatgcacaagggaaaatgatcctgttcttggtcctgatgggaagacgcatggcaataagtgtgcaatgtgtgctgagctgttgtaagtagcatcatccccaggtggacttgatgatgatgcacttggttgctgtcccgagaatcactcagcagagagataaaatccttttcatgaagcatgcaattctttgtctttacactgtgaaatagcctttctcacagaaaggcttcttttctttttcttcttatcattgattgaagtttcttaaaagaagagaataaggtggtcatgttagttattaaattcaagactctcatttcttttaaattacagtaatttgaaaatgtatagtgttttataaggcacaatattttaaaatttagaaagcactttcatatggaatgatctaggtcagtcgtctgacatccattgcttcatttgggcatatgtagagacccttcctgagtactttagagcacatctacaaannnnnncccccgcatgcgatgtat

SEQ ID NO: 8 - DNA, 965 bp, Homo sapiens
cagcattattttaaagctcttggttctgcctgttaaagggaattttcctgacaatcttctgtttgtcaatcaaggtgtcacacccattcagttttacagaacaaagcttctaattgactgtcagtgagtcagtttcacaccctttcttactataggcaagaaagctaatttacagaaagcaagtttcccagataagctattgccgtctttctttctttccttatctttggcaatttctctggctcaggcattgaagacggaaatgcttgtctcttgtaagaggtcattttcagcaattcgtagcagaggatatgaaagtgtttagcacaggactgaggcatactgaaaatatgattgagtcataaactgaccaactgtttacttttcttaacagtttaaaagaagctgaaaatgccaagcgagagggtgaaactagaattcgacgaaatgctgaaaaggtaaaatgactcaccaacgcaattttgttcttgtggccatatttattaattcaaatgtatgcctaaacctttgagtaatgaaataaagtcattgtcttttattattatatagatatttttcttaatatcaaaattccccaaattgactattcctagaatattaatcattagattcagatttaaaacagcatttcctacctaaggcccaaaatgtgcagtttccacctgtgtttctccagtcctctagcccccaaaagaagatataagatagaaagtttgtataaaccaaattctgcttaatttctagcagccttctttttggtttagtgtaaacccattttactctgaataaattgtttaaaattggcaatgaaatataaattaaacaggaataattttaaaatagtgagagtgttttaaacactatatccactagtgcattttatgtgcaattaaattgacacaagttgaagttattcactttgccattgtgattattctgttcag

SEQ ID NO: 9 - DNA, 1621 bp, Homo sapiens
  misc_feature (1016)..(1016): n = a, c, g or t/u
gtgatttggctgctgcattttgttgtgtttgtgctggaacctcatgaaagatatcccctgacccctattgatctgaaagtttaaaagaaatacctagttttaatccaagaaaactcttggtcagccttattttttccgaggctgttttttatcttttaataaagctaccacataacagatacaagtgccaagtagttgacaatataattactgtttcttagtaatcctcattaatatgttgtatgtatttgccaacttctagacctgatgttttgtggtaataaatctattataaagtttccagattaaaacgtagaaaaatggtaaataaaattacttgttggctggatgtgttggttcatgcctataatttcaacactttgggaggccaaggcaggaagattacttgagccaaggaattcaagaccagcccgggcaacatgatgagatccagtctctaccaaaaaggaaaaaatttagccgggtgtggtggtgcatgtctgtagtcccatttagttaggaagctgaggtggaaggatctctgagcctagaggttagaggcttcactgagctatgatcattcccctgcaatccaccctgcgcaatgcagcaaaatcctgtctcaaaaaataaaatatcctgaaatgaaatgaagtaaaataaatataattgtcttttcaaaggatttttgcaaggaatatgaaaaacaagtgagaaatggaaggcttttttgtacacgggagagtgatccagtccgtggccctgacggcaggatgcatggcaacaaatgtgccctgtgtgctgaaattttgtgagtatagaagtggttttttcagagtgattcaatgggtgggagtggagattgattggattgatgagtaaaaataactttgaaaggaagctttgttgttgagaaccatctgagcagttttatgccctccacaaatcataatgccacctagtgagcaggcactactgatgtttgctatttctgaagagaaatggattccattttncagttgaaatactgcttcagcatacacactttttaacattcccctgtgactgtgacctatgtgaaactgtttgtgaactaattctantatanaatgggggtaaaagcaggtaccacgacttcaattgtttttcttcagnaattatcccatggtacaagngttgatgccacactctcaaatccttnanggnttatacattaaatngggatacccctnatcttttttatctgggaaaatattaacccttgggggnaanggggnaaattttnncaacaataccaaantttttnccatttttttaaaaaatgtttttaaaaanaaaattttattgaaaacnttttggggttttttttaggttttnnaaaaccctttttnttgggggcccgggccnnttncccaantnctttcccnaantttttggcccccctttttatttccccccccccttttgggaaanaaaaatttggggntatntttttnttgggnaaaaaaacccnaaaaccntncccattgnaaaaaaaancctaaancccnnaaagggggtttttaaanccccccaaggggggatanaaaaattttcccaaaattnnggnnncncnccnttt

SEQ ID NO: 10 - DNA, 946 bp, Homo sapiens
  misc_feature (5)..(5): n = a, c, g or t/u
ttatntaactttggtttaacttccgtggcagagccctttancntggttttctgnaanccaagccccnannaaaatttcaacccagaatgttttttacccccnnccaggggcattaaaatttttncccagatttgtgcttccgtntttnnccancncaaaantcaaaaggcagggtagggttgtattnaaactcggaaggcccagggcccagccacccttgccaaaaanccatattactccctaaatgaatgaatgaagatatgaatgaagatctcccactctcaagaagcttaagttcagtagcagaaataagccatggacatgtgcacatatttatatattgcaaaatagaataaaaactgctgtataagagaagactattcgatacaaaaacacagcaatagacatagcgctcgtttgtatttgggaactggaatgtcttcttcaaaaaggtagaatttaagctgaagagttttaaatttcttttaagaaagggagatgagaaaaaagatcattttagtggaagtaaatggtataaaggtgaaattaaagggtctttttggggatttgaggtgtttttaaagtgtttgtactaaaactcaggacaacttagatatttttccatctatacctaatgactgttttgtaacatgaagatcggaagcgtctctactcatttattttactttttccagcaagcggcgtttttcagaggaaaacagtaaaacagatcaaaatttgggaaaagctgaagaaaaaactaaagttaaaagagaaattgtggtgagaatcagtttgatcagtctagttacaacttgtgtgtgtgtgtggggggtgcgtgtgtgagagagtgcatattacatagatgcactttcaatatgtttaatatttccacactactaataggtttgctggaactagtttctagttattattttatgaaact

SEQ ID NO: 11 - DNA, 38653 bp, Homo sapiens
  misc_feature (1846)..(1846): n = a, c, g or t/u
cttttttggcgatgtgctctaaagattcgttgaaaatgtaaatggatattacaaggagagaaatatcacaatttttggatggtcctaaatcttaaaagttttatttttcatccttaatttctcttttttctttgtaaaataacatttaacattcatacatagaaaacagaactatatctcaactttttcttattcattattcagaaactctgcagtcaatatcaaaatcaggcaaagaatggaatacttttctgtaccagagaaaatgaccctattcgtggtccagatgggaaaatgcatggcaacttgtgttccatgtgtcaagcctacttgtgagtatagagttttagaatgtcaaagaaagaagggatcttgcaggtaatttaatagaaacagcttctttcatagatggggagactgtggctcaagacagggaagtgagttgaaaacattacatggaaaatatcagtgatagagctgggagtaagtacctagagttatttgttttaagacgccttgttcctctgacactccctcttttagcactggaatgtcctgacaaacatgaacttgtacaaatagtagatgcccccttactcctgaaacttcacacgttagcctgttttagcaatttataggtatctcatcttccatgggagttaagagtccaaaggtgatgcttaatgtagaaatggaataaagtcaaaattcccatgaaacaacactaaactcctataaaattacaatgggtattattttatgtaatgcaatatttctcaataagttagaatggacagttttctttcaagatttacgatagctctttatttggctgagctcaaattaagtatttaacttatatttagaatctatggctttctttgttttgattgacaactacatatggttgaaatatcatcagttaactgtacatactgtgcaattccattgtacaaagcaaatttaggcttatttgttttatcagtatatggaaattattgtggttttttttttcacatcttgtcctttagatgaaacagctatgagtaagcaaaatatttaagtttaaagcaaagctagggtacacatggtgtgatagatagaccatggtcttgagggaggagaccttggttgtagtcatatcctgagatgtagcaaagaccccctctacctctcatcttctctgattaacaaggggatgagtagatgtgctttgttaaggccactttcagccttcaggttatgccaccatgattttagttttggtgcctctgttatgaacattgatcatgctccttttcttactatgggcaagaaaactaatttacagaaagcaagtatcccagatccactattgccgtctttctttcaacattaaacagacattatgaagaaatcatagcaccatactatcctggaggatattttgttgcttctcattgatatgcagtgataaagggacaaaattgttccactctaaggagggagaacagttaacagtgcaaggatgtggagaaatcatggcatgtgtttgttcctaatggatctgcttctttttccctcttattcagccaagcagaaaatgaagaaaagaaaaaggctgaagcacgagctagaaacaaaagagaatctggaaaagcaacctcatatgcagtgagtggaatccatccaataaatcctatttggtgctataatttgaacaaattttaagagcctaaagggtgagattttgccctgcagaaatccccagaatatcttaactcttcaatctggggatggtattgagatgaattatatgggaagattgatccattcttgctgattaaaaactaactctgcaaaaaanaaaaaaaaaaattgtttaaaagctgaaaaactgagttctacttctagttttatcacttatagactgttagcttttgctaactactttccagagaaattctacatgtatgctttctacatatgagtttctgatatttcacatctaatttgagataaaaaataccttgattcccccaccaacgcatgatttttatccctgaaatattacaatattagtttccaaagccttagctcagttatcatcaccaatggttttgatgataaaagtctcctaattttcactctggattttatcctttcccttgcccaaccagtctccctaacatagttggagtaatgtttcaaaatgtaaataagatcataatattgctcttaaaacccacaagcagctcatcatcatatacaggatatgctttaatcctgttatctctcctcttatttgtatccttatctccattcagaaaatcatgctctgctatgtgccatgtcttttcctacctgtgtttttcagctagttttattttcataaacctgtgtgcgtatttcccaattcacttttttttgcccagtcaaatcctatttacacttccacagtctcacctaaattgttcttgctccctgacaccttccctggtttcctttgagttctcagattttattgtaccctgtatgttcgtacctattttattctgtttagattatagtggaatttgcttatatattgcttgtattatatggggacattgtgctatgttttattttttcctatctcttggcatatgatgtttttcttgttgtcaaattgaattttacatttgagaaaaacacaattaaaatcctcagctcaaagagatgtaacattagtttctgccaatgtagatgtttgaaccttctgctttaagtagaaatgaaatatatggccaacttacttcttctatctcggcaggagctttgcaatgaatatcgaaagcttgtgaggaacggaaaacttgcttgcaccagagagaacgatcctatccagggcccagatgggaaagtgcatggcaacacctgctccatgtgtgaggtcttcttgtgagtagccctgcagctgggaacatggaggaatgattttgttctttctatttcatttccatgttcaattatgggagggccacttcaacataaaaatgaaagaattaaggcatatttagagaaactgtctgattggagacatgtatttgaaaatccatgtcctttgaagattgtcaagacttcacacttaagcaaggagaaagtcataatcttgaaatattcaagtagttttgtcttttaagtgtggaaacaggacaagttctagcttatctccgtgaataaaagtgaggccagtgggccaaagaatgaaaggtctggaagacaggttccaagtggatgccaggaagaacttcctaacagtttgcggatttcaaagatggatgatttaccatggaattccaaggatgatatcagtacagactttacaggcagattaaagcaaaggcttgggagtcattagagatacagtaatgagcattggaaatttagaaaaccacctctggactagggtttaaatcccaagtctgtacttattatttctatatcttcaggtgagttatgcattccgtctgtcagtttatttgtatgttggggttataataatagttttgtaatgcaattgtgaggatttcacagtgtaagcacagggttaggcacatcacattcaaagaatttaatcgttgttaagtgtaaaattaaattatatttgagatcacttctaatgtggcgattctatgatttttacttatctcttcttaaccatccttttttagccaagcagaagaagaagaaaagaaaaagaaggaaggcgaatcaagaaacaaaagacaatctaagagtacagcttcctttgaggtgagtttatatcctccagcaactcagagggatatggccctgaggatccacagatcatgttcagggaacacgtgcattccttaaaactgtatgaaacaattgtacagtttgtgttttcatatgtgttttcatgtgtgcagttatacatgtgcatatgtgactatttcttggttgttgggttcattgtttttatcacttaaaaaatccatgccttcaaagttaatcattctaaaataagccaattgctaatcctcgtttaagaaaaaaagtacaagctttagctatttttgcaatctgattcagcctatgttcaacactttcaacttcctgcctcaatttcacaggagttgtgtagtgaataccgcaaatccaggaaaaacggacggcttttttgcaccagagagaatgaccccatccagggcccagatgggaaaatgcatggcaacacctgctccatgtgtgaggccttcttgtgagtagagcagtagccccatagcgtctgaggattgagcagtgggaatttccatgggaagttttcaaaccattgtgaataatgcattttcttcttcagtgtagcattcagtcttgggtgtcatatttaaatggactaaaaacaaacaacagcaacaataaaacaagtggagggctttctattatgatataaagagatgcagaataagccagacacagaaaaacatataatgtgtgatgtctcttatatgtgggatctaaaaaaagctaactcatagtagagagtaggatggtgcttacaagagaatgggagtagggaagggtgagggttgggaatgaggaaatgttaatcaaagggtgcaaagttttagttaagcaggaggaataagtttatgggatgtattggacagcatggtgactacagttaataataatgtatcatatatttcaaaaacattaaaaaagtaaatcgtaaatgtccttacaccttcccaaaaaagataagtatcatgagacaactatgtaaattgcttgatttaatcatcacaatgtatacatataccaaacatcacactgaattcaattaatatgtacaattattatatgtcaagtaagaacacaatttaaaaaagtgaggtgcagaataatttcttctatgacatttaaaaagtaacctagaatcattcacccagagaagagaatacttcatgataattatactaattgcttttagttttatgaaaacattatcagatgaaataggaaatacatatgattacacataaaatattacaatgcatggaaaatttatgggatttttttgttcactatctttcttcctacttattctatgtgattttaaggaatataattaaaatggaaaagagctataggaagaaagatttgggctcatgatgaagattaactttgtaataaagcagaaaaagacaagaatggagattttattatgtaggagttgggagcctattgatagggtgcctaaaataggtctttaggatgaatgtcatggtatggccggcccttcaaaaacgtgactaggcttttcttaaactcttgatttttgactaaaaaaacccccattttatttcccatgtgccaagaatcttagaaaggacttaatttatttagtcttcccatatagaaagcaatataatccagcatattttcccatagcatttgaggcttggagcactaaaaatctattagaaggaaagttgagatcatgaccatataattatcacaacaaacgtttgttgaacgcatataattatgtgacacttgctcagagatttgttgcctaatctcatttcatcttcacaacaaccatatgaaatagatgaaaccagataaaaagcaagaacaaaaaacaaaactaaggcccagaacagttgagaatgacggaaaagtcaggacaggatatgggacttacagatgaaggaaaagttaatgtatttcttgttgtggggtataatcacttagtgaagtctttcagcctgttttttgttctcccttacattaatttttcagtagcaagaaactaattctgccacttagtggtcatctttgctgcctcctctgacaattttccattggggaaagaaaatgctgcttaaatgttttagtcaacttagactcgtgataaaggttagatcggttttcatataaagatgtatgaacaatcccataattttagatctattttagtcagtttatttcataagtatataaattctttattgaaaagaaataacctttatcacctcatgttttatagatatgttaacaaaggtgattgattttttttgagaattattctaccctccatagttagtttttgcacactttcataccatgtgaaaacagtcttttcaatttttaaaaaagttttagtttgatctatgtgcaagtattttgttgattgggacacattaaaacattttcacaaattatgtaatttttttctacattttgaactatttgtgttatatatccaattatgctgtagatccaatccaccaagctcaatttgcaacactgttagttatccaaaatgtaacagctatcactttcacttcatttccatcatatgcctagttttggaaggaaagtttcctttcaataatgacaataatgattatgtcaatctcttataacaattatatcaattattttattttgtaccaggtactgttctaattgctttacatgtagcaactcatttaatgctcttaacattccaataagaaaggtacttttattacctcttctattcctcctaaacacacacttaaagatgaggaaggcacagagaggttaaggtacttcttgtcttccttgcagaatttggctccagagtctttcctcttaaccaccatgctatactgcctctcagaagataaatattacctctccataaagagtaatcacacatgcaaaagaatcagcaaataagaaagtactattgcagtacatatactgggaagttaaacaggtttactccctaattcaggggctttctcttgcacaccattctcttgaggtagagtgaatcataattgtttattgtctaataaaagtataaactactatgggtggatatttggcagtgtttgagaatagtcaaaacaataaatgatcaagctatgaaagttaaggatctagtctactatattaagatccatttctggccaggcacggtggctcacacctgtaatcccagcacttttggaggctgaagcgggtggatcatgaggtcaggagttcgagaccagcctggccagcatggtgaaaccccgtctctactaaaaatacagaaaacttagccgagtgtggtggcatgtgcctgtaatcctagctacttgggaggctgaggcaggagaattgcttgaacccgggaggtggaggttgcagtgagccgagatcgcgccactgcactccagcctgggtgacagagagagactccgtctcaaaaaagaaaaaaaaaaatccatttcttctctctttttcttttcggtttcttaaagtcaacaagaagaaagagcaagagcaaaggctaaaagagaagctgcaaaggtaatattctcaggaatgctgatgctgtgccctgacatttttcatttctttataattctttacttatcctattagaataaatgttttctgtttaattttcatgcctgaaataaatgaagaacatttttaacctgaacaaaaagggtcctgataagaagtgtccagagtaccaattttatttcacaattattcaaattttgaggtttatttcataaaaagtggaatgttaactttttgtaaggaattttccccatatttggcttttattagatttctgttgttagagtattaacttatgccatttactaatttcaatttacattattcatatctctgttatttcaaatagagagtataagatctcaatcattccaatttacaaaacgaaagcacatatattatcatatttgagctttgtcaccacccagctatggagcaagtggtggttttcttgcacccaattaagattattaagttgggcctctgactttccagttatttttattccactctccatgcttttataattttatacatagcagagtcaaatttatctgtgtggagatgttgaattattctcatataatagctacatacaagatttatctttctttctttttttctggtttttttgagatggagtctcgctctgttgcccaggctagagtgcagtggcgcagatctgggctcactgtaacctctgccgcccgggttcaagtgattctcctgcctcagcctctctaatagctgggattacaggcacgtgccacaatgcccagctaattttttttttttttttttttagtagaaatggggtttcaccatgttgaccaggctggtctcaaactcctgacctcaagtgattcactcaccttggcctcccaaagtcctgggattacaggtgtgagccactgcacccggcctgtttattctatttcttaacaatagccacaacagtacaaacctcatcattacttggggaaaagttgacacagtttaaaatttgccttcttactgactcttttttttttaaataagtgtttctactaggtgaaactatcttttcattgatcatttagaattgaaaatgtgttttcatccattatgtttgatcagctgccaaatcctcttagatatttccagtggtattaacatattcctccttctctccactcctattcttaccctcttgcttcaaatccacatctcccattcctggtttttaatttttttaaatcaatttactcccagaaatatgttagtttctccagggatcagacttattctcttctctatgtataaagatatgtctttttaaatttatttatttatttatttttgagtcaggtcttactttgttgcccaggctgtagtgtagtggtgcgatcaaagctcactacaacttcgaactcctgggctcaagtgatcctcccacctaagcctcccaagtagctgggactacaggcacacaccaccatgcctggcaatttattttatttttttattttgaatttttctgtagacacagagttttgctattgtgtccaggcttgtcttgaactcctgggctcaagcactcttgcagtggcctcctaaagtgttggaattacaggtgtgagccaccatacttggacaaaatatatatttatttcttaggttaagcatgttttttctcttctttctcagacccctaaagacaaatcacaggtaagcaatattctcatttcctccaggaaggcttctcatctaagcctgcctatggaatcttcccacctctgatctttcctagcttttacagaccatgctatgcaacacaacacacacgtatgtttttgatgataaatattttcagatctgctataatgatgggacaattctgattgatgacggaagctttgtaaatacttactgatttactactatgtgttatcacttttgtttttactttttactctacgttacatattcctcataataggaaaagatgcaggaaggatagaaaactactctgagaaaatattttcttcatttcccaggaaatctgcagtgaatttcgggaccaagtgaggaatggaacacttatatgcaccagggagcataatcctgtccgtggcccagatggcaaaatgcatggaaacaagtgtgccatgtgtgccagtgtgttgtgagtgtccaccccatctctcccactgaatttcttcatccatgatcgcccctgagtctcagatccttcatgcatgtgtagagtatagaccgtgagttatatattagaaaggtttacaagcagatggataagacattaagtaatattgaatcatatagtgacaatactattatttattcaattatccttctattttccaattaagtcaagggaaaaagctcaatttaatggtttttttttaacacttgataatcttcatttgtgaaaaagagagagcagaaatttcatgatttggattggatgatctttttgatgtctttcccactcctgacattttatgatttgttagtctattcagataggagctaaggagaagccagctggatggaggtctgtggggtcagtacataaagactattaaaatagcctgaacaataaatggaataggaaggattgttatttatcctttcaaggacattactatataatgcaaacattcataagtggtttagtgtggagtaaaacctttaggaaacaggctcataccctaaatgtttactaaaatgtctccttgttaagccaggttcatggccttctaaggtctttgttttgctgagtggtgtaggtaagcctgtgactacagctccatcgtgtgggacaatatagaggttaattattatcacttctaattctcctttttaggctcttaatctgctcctgaacttaaccctaaaataaatgccgagaaaaacgcttatttaaataatacctgctgaggtgcaaagttaatgtctcctaactttcacatgtcttgatccaaccttaagtggatgcatacaattccctaaaaggagaggttgacagagtagagcactagaagaaaagtggcctgaaattcaagaagccagggtttttctctcacccgttcaatttgctggtgttgtggtaatgactaaatgtagtaaatgctaaggattttccagcattaacatgctatatgtcagtagaaaatggcaagaataccagaatattaggcttgtgttcaacccccaaagattagcagttgaatctaactggacttaaatcactactgcaatctaaaccttgaatttcaacttcaatattagctccattgaaataatatttaaaatttttagtttaaagtaaaacatggtttattatgattttgagcccatcatctgacttagcatcaccctttgcctttatcacggatatggtagttacttggagaggaaaggtttaagcagtgaaataaaatcaagacaattgtagctttattcggaatgttaattaaaattagcgacgtacaaaaaataagatcacctgagggccacagactgataaagatgcatgcttgttttgtaattattcttctttagagggggaatgtaaacagctaagtttatctgtactattggttaaagcaaaagcacctctcagactagataaatttgtattgaagactgaatctgactgttggtttggaagatcctcattccttttaaaaatgtactaccaaaaagagtagggaatcatattcagcctatatcttctttttctattacagcaaacttgaagaagaagagaagaaaaatgataaagaagaaaaagggaaagtcgaggctgaaaaagttaagagagaagcagttcaggtagttgtttgagatcatcagagccacataaatattcaacgatcactctccctagggagggctcctattccctcctcctcattccagtattcttagtttctttgcaaactgggaaaatgtgtttggatccccatatacaagagtactcccctttccttattcaatttattcacattaaatctattaattgctcgtgcaggagagagatcaatcaccattttctcccctgattgtgtcaattatattacctaagaaagaagtatatatagattgttatcttactgtctgaatgcagactaatagagacagatgttcagatttttctcaagtacatttttgaatatgaatatttaccttaaaaacagcaacaactagggatcccacttaattatctgtgagattttgaggagataaaagcatctctgagtcttgagtttgcattctttaacatctcacatgatggtgataacaatgataattgtacattcgttgctactgtatgcatcatggcataaagttttatgactgatatttgtagtagccattgaagggacatattattatctgtgttttatagaagaggacttggaagctgacataagaagttaagaaatatatttaattagttacatataatacaattatatactgatatattatttactatatattaacataaatattatatgatacaaataatatcatatataattataattatatgtagttagtaacttgagaaccaagaattgaaccaaaagttctggctttaaggcccatgatgcactttagattttacacgcttctgccatttgtattattcctgcagactatgccttaggtcctcattattcttttcttttgtccttctgtgatttagaagagctcatttctactgctcttacctttgctctctacaatctactctcaactcttaagccaaagtaattatttaaaaccataaggcaggtcgtgctacccagtggcttctcacctcactcaaagtaaaaatcaaggtccttgtagtgtctggaagtgccatacatggtctgatccctgccacttttcataggtcacatttcctattattctacattgttctccatgcaccaattatactggccaccttttggtttctgggacatcccaagcattgattcatggctttctatcacgtggtcacaaaaacaatgtggttaaaaaactggactctaggctcagactgtcagggttcaaatccttgcttcatttccctaccagtaaggtcctacagttaacatcttcatatattagctttctcacttataagataaggacattattattacgtatattatttttatgaagtcccaagaaacttattaatgtataacagcacagtgtcacacatatcttaagtagtcactctttaatattatatttattgctgttaatgtcattattggtctaatctcattgtccagtggggtgttccatactggagagttcaccaattcttcaacatacaatgaactttaccttctatatgccattactcacagtcagaatatccctcttgtctaccaaaattctggccacttttcatggctcagactgtgtgttaactgctccctaaatattttcccatgtgtgcaatatacttaataactctatcacttgttatgccaacaatttaaaaagtattttagttatattattcattgtattataacttatagttgcctctatgtgttttcttcaaagaaattaaaaactctgtgctagttttctatggttgcgataacaaattagcacactttagtggcttaaaacaacataaacttctcattgttcaactcccacttatgagaacacatggacacagggaggggaacatcacacaccggggcctgtttggggactgggggaaaggggagggagagcatgcaaatatctaatattagccccctcatgcaaatatctaatgcatgcaaatatctaatattattagccccctcatgcaaatatctaatgcatgagggggctaaaaacctaggtgacgggttgataggtgcagcaaaccaccatggcacatgtatacctatgtaacaaacctgcatgctctgaacatgtatcccagaacttaaagtaaaataaaacaaaataaaataaaataaaaaacaatgaaaacaacaacaataacataaacttcttatcttatttcataggttagaaggctaaaaaggatctcactgggctaaaatcaaagggttggcagagctgtattccttttctggagtttgcaggcgacaatccatctctttttctccaaatctagaggccgtccatgttccttggttcttggaccccccttctctgtctacaaagccagcaatgttatatctctctgactgttcttctgtggttacatctccctctcactctgtcttccatctcgctcttgcagttttaaggactgatccttgtgattgagccttacactgagcccattatggcaatacaggataatctcttcatttcaaggttattaacttaattatatctgaaaagccctttttgccatgtaaggcagcatattctcaggtctcaagtattaagacttagacgtatttggtggaacggtattattctgtataccacagattcctttaccaatttaagaggccatacatttttcttctctgtatctttcacagtgcctagtgcagaataatatttgacctgaaggttttctaatgagataaataacagcaatccatgttccaaatgtgtgacctagttctctgaaaagcattttggttactatcaaccttcagcatcaccttttccatgcaatcattatgttttagtttttaaagcagtattgtggaggaagatttctagtgtttagttattggactcttaaaacctgcttctgcttcatttggcaggagctgtgcagtgaatatcgtcattatgtgaggaatggacgactcccctgtaccagagagaatgatcctattgagggtctagatgggaaaatccacggcaacacctgctccatgtgtgaagccttcttgtgagtgggcggcagccactgctgctactgagtgtgggagaagatcagcatcgggtgggcaagaggggtgacattggaagttttctccaggagatagataataaaggctgtctttgcactgagtttggaaatttactattataaagccatatattcagcccatttatttctggtgttcttcaatagtctgagaggtacttgaatcaccatcaagaaaggaccatgtgctaagagcaaaattacactggagttaatggcataaaaggattaggacttctgttctctgcaagcataggtcactgtcaaataatccaaaggtgtaatcattaggtgctaataaattgaaaatgagttaacatctaattgatatcttagtttatttctggggagaagaacaatatctttatttttcaaaagagatatagtcataaaagtttcttcactggtttgctatggcatgtgcgtcttattctttaaacaaccttaagaacaagaacggctgggtgtggtggctcatgcctgtaattctagcactttgggaggccgaggcgggcagatgacgaggtcaagagatcaagaccaccctggccaacatggtgaaaccccacctttactaaaaatataaaaattaactgggcatggtggtgcacgcatgtagtcccagctacttgggaggctgaggcaggagaattgcttgaacccaggagaaggaggtttcagtgagccaagatcgcgccactgcactccagcctggcaacagagtgagactccatctcaaaaaaaaaccaaaaaacaaacaaacaaaaggacaagaacaatgatgtagagagatgtgtgtttacctttccactccaaagctaagttggtggaggtatagaagtaatggaccatttttatgtagagacatttctcctttagggtagtatgtattgggtgctaggaatgattgttttgtcctcccttttcttatagccagcaagaagcaaaagaaaaagaaagagctgaacccagagcaaaagtcaaaagagaagctgaaaaggtagtaatcctgaatgtttatactgcaatgaaaggatgagattttgcagtatctctgtaaaaataactatggaattactgaaaccccagttgtgagggaactaggggttcatttagtccaggggttgagaaactttttcttaacagaacagatagcagatatttctgtttttgtggccatagaatctctgtcacaactactccacttggctgttgtgactgtaaaacagccctagactgtatacaaacatatgggcatggctgactccaatacaattttatttataaaaacaaacggctggccagatgttgctcacaggtcacagtttgcaaacccctgatttattctgttcccatatattaatattaagatttaaaattcagagaaaaagaagaacttgcatgtaaagttaatgggagtatgagtatcagtaccaatagcttcatatttcaaagttctggaacaatagaattcaatcctagacttcctgataccacagccagccttctttctagtacactatttgtttttggattaccacatttggtcaacacatgtgttccttgctttacctgtggttatattttggtgccagcatttaggaaaaagattaaaaagtagcatccccaaataatattttcttggattcaaaaaggctggctccaagtagtttattccaagttgtggtcatttccctgcccccatcaaagactattgagattaaaaataacctctaaagaagtatcgccctggaactctgtatgtttggcttgtttacagttgtattgcacagttgtaataattctaaatttaaaaataatagcagttcatatgaaaaatagattcactcttcaacatctacaagagaaaattcttcagtatgagctaatttctgatttcacattactactacagacaaactctcctctcattattttttcttttgctaaatataactagttaggggatctggcaatgaacctttaagcagagctaggagaaaaattccctctttcatcctcattgaatttcaacttgaactaaagaattcagaataaaatacaaaatcaattgtaattgccatttgtaattaactctttgaacttcagtgattgcttattctgtctcaatttctcacgtaaccctaaacaaggagtgtgcccagtcatcagagaactaagtttattgagagtcttgtgtatggttgcaggtttagtctggattttggggctatcaaggaaaaagaaatacagccaccttcttaagtcctttgtgtttaatataaattgaacactataactgccagaaaaaaaattctcaggtcattttctctctcttttttttttttctataaagcacttagtaggaacccagtaaactaatttcccagaagatactcaagctttctccttttcttttccttttaggagacatgcgatgaatttcggagacttttgcaaaatggaaaacttttctgcacaagagaaaatgatcctgtgcgtggcccagatggcaagacccatggcaacaagtgtgccatgtgtaaggcagtcttgtgagtgcacaaagaaaaccactactgtgggatggtggaattggggaagcaatgagccaggcaaataatatgtattgtgtttgtcctttccttacatgtaatagaaacagaaggttatctgtaaagcatttgaaataaatcttttctttataatggccaagattcacagttctagaaataaaataaatacaactaagctattacattctctgttagttctggggtagaccctgccagtagatcacacttccttgataaaattcttaagttatttgctatcaagtggatcctttctctggattatctcgcaactcatgttggaaagaaaacacacagattttaatgtcaaagtccttacctacattgttgaaaataacacattttcagaaaatgaaaagtaggtctttacacaaagtttggaagtacacctagaatatttaagtccttcaatagcatttaccaaacatctacaatgtacaatgtgtataatgtgtataatgattatacaaaggtgaatgaaatagataatcttctcagaaatcatatagtccactgatattagtcacagacgtaaggtaatataatacaaaggagggactgttgaatgaaaaatttgtctgtggtatgttacagtacttcaagaaagggataattaatagaaagttttatggtgggataaaactagaccttataatttggataaaatgtggacatgacagaaagtaatgagaggagaagaagggaacagcaagagtaaggatctaaaattaggaaaaaaacaagtcatttagtttagtcagaacttaccaactttatcaaccttgaatgcacaccagagatttcgaggtcaattctacatgtaatgggaagctgcaaaagttgttcagataggacagtaagtggcataaatattgttcctttaggaagattcatttgttagcagtaaacaggagaaaaagagaaagaaagtactaaacttaagaattgctactcatgtagagggaggagctgcaataaataacccaggaagaggcagctacgcagagcaaggaggtaggctagaaggcgagtggatcaaggtgggggatgctgtgaaggtgaagtcaatgtcatgtggtgacagctatgcaagctgaaggacagtaagcaattagagatgattccactgttttgtgtttgtttttttgtgaaaggtagaaaatccaggagaaagaatacatttggtagagaagataatgattttggggcctcttgaacattaggtattggcgataaacacagctggagatccccagtgaacatttatgaagtgttagttaattgctcaccttgtcttttttttttttttttcttatggagtcccactctgtcgtccacactgaagtgcagtggtgtgatctcagctcactgtaacttctgcctcctgggttcaagtgattctcctgccccagcctccctagtagctggggttacaggcatgtaccaccacgcctggctaattcttgtatttttagtagagacagtgttttgccatgttggccagactggtctcgaactcctgacctcagggatccactcacctcagccttccaaagtgctgagattacaggtgcaaaccaacacacccagctttactcaccttgtcttgtttatggtgatatatatgtggtgttcttttattttaatgagacccttcctggcaaaggactccatataatttatatttttatttttagagaaggcctgcagcatagtagatgttaactcaatgctcgataattctgtacatatgtgatttgttcatataatgagaaactcaaagaaatgcacaccactctctgtaatctattgttccctacctcccactttctaatttccagccagaaagaaaatgaggaaagaaagaggaaagaagaggaagatcagagaaatgctgcaggacatggttccagtggtggtggaggaggaaacactcaggtgagagcaacctctaatttcagtaactggggcgggctcaactccttgacaataggactgtgaatagcgtatatagtctatggtaaaggcagtgctaagtccttgagagaacagagaagtttctccttaaggttgcagggtaagatatcagcacattttcactgctctggggttcatttggatttctttacaccttgaaataattaacaatatgtaatgaccttatcccagtctcctgagccctctttatggtaatctagacctcatatcaataatcgttctcatcagtatcacatgctcttcattttctttcattattctttcttgccttggacagagccactttttttaggtgtcttttctgttgatccaaggcagcatgataggacaaaagagcacaggctttggagtcagattgggttatgattctgctcctactacctggacttgtgagacattgagtaaattacttcctctcactgaattctactttccctgatttagcagtactcttccagggataaaagaaaataatttaggcaaaggtttagcatgtttcttgaggcatttttaagtgtcagcatggcaatgttgcacataaggtcactccttgggtctctgaacacagtttcataaaattgcagtcgccttatttatctttttacaaccttggaatatcaaaaccatgtcttcacttccagatctgacaaaggcccaatatttacctttgataaactctggctattctgtttttttcccatcttttttgctttaggaggctgagttctcccttttaaggaatagccctgttgattagaattgtctgatagacattttgtgtgtcttgttcataacttggtcaaacctaccttccttgacattttgcataactttgggttttcctccagcatttttctctctcttctatctgcactagagacctgcacatttttttctgtataaggccagctaaatattttaagcaatgtgggctttgtgggtcatatgatgtctgctgcaactattttactctgccattgttgtgggaaagaagcaaaaaaataatatctgaataaatgagtgaggatgtgtttcagtaaaactttatgtgtaagaacaggaagcaggaaagatcttccctgtgggctatacaattgaaccttgaacaacatgggtttgaatttcccaggtccacttgtacttaattttttttcaaccaaacttagataaaaaatacaggatttgtgggatgtgaaacctgcatatatggagggctaactttttgtataggtgagttgtacagggccaactgacagacttcagtatacatggattttgatcctggaaccaatcccccaagtgtaccaaggggaaactgtagtttgctgacatttaatatatgctattaaattcagtcatatttataatttccttgtgtagatccttaaaagcaacattagtagccatgacaactcatctcttataattacaacatgggtgattctggtccccttttctctgcagatgccagacctagatgttaggggtttttttgtttgtttgtttgtttgagatggagtctcaccctgtcacccaggctggagtgcaatggagcaatcttggctcaccgcaacttctgcctcctgggttcaaacgattctcctgcctcagcctcccaagtagctgggattacaggcacccgccaccatgcccagctaatttttgtatttttagtagagacagtgtttcaccatgctgtccaggctggtcttgaactcctgacctcatgatccacccatcttggcctcccaaagtgctgggattacaggcatgagccaccgtacccggcaatgttatgtttcttataaagagaggtgaaagaacttctcttactcagactgttaaaacaatttactaagaatacagtagactaagtaatccaggggctcttcgttcttctctgttttcaggacgaatgtgctgagtatcgggaacaaatgaaaaatggaagactcagctgtactcgggagagtgatcctgtacgtgatgctgatggcaaatcgtacaacaatcagtgtaccatgtgtaaagcaaaattgtaagtatttctctcaacaggcatgtctaaaatatagtcacattcctcttgaatgaatggctatttctcatttgctatggctccttccatgcaaattattctttttgctaatttccaaatattctatttttttctcttgcgttctctaaggaacaatgagccttagcaagggcagctaaccaagtggctgatttctattgtattttctattacaattctgtgaactctaagtgctatcatgatagacccttgttctttattgtgccagaatcctaggaagactttgtcacggccatttggaatcattgaccagcacagttgaagaaagctgacttcttgtcatttgcacgggacacacttagtgcataatccagggtcaatatttgttaacaagatgaatattcacttttttctcctccagggaaagagaagcagagagaaaaaatgagtattctcgctccagatcaaatgggactggatcagaatcagggaaggtgagttattttttgggttttggcaagaatcgtctttctgtgagtcagcagttggcgatcaaaactatagaagaaggtgtcaacatgatcaagctagagcttgcccttagaaggtcaggagttaccaagttatcagtgactctcactgataacttgttcagtgaagaaacctaatgccaagtggacatctttaagtctgacactttacaaggcatttttataaattacagtttatttaattatttcaacagcctggtcaaatttggccttttaaccttatttcgtagatagggaaaatgaggttttgcaagtatactgggatttatttttgtttttgcattattttgctgctagtgtaagttactataagcagatgcgtaaaataacacacatttattatcttatatttctggattccataagttttaaatggtctgattgtgataaaaccaagatgttggtaatgccgcattcctttgtggaggctctaggtgaggatctgtttctttgctttctctaacttctagaagctacctgtattccttggcttgtggccacccttaagccagtgatggccagtcaagtccttcttatgctttatcattctcacactgactgtcctgccgctgcctctctctttaatgagtaagaacctttgtgattacatgggccccacttggataatccagggtaacttccccattttaaattcaactgattagcaatcttaattccatattcaaccataattcctccttgccacgtaaagcaacatatttggaggttccaggaattagaacatagacgtctctctctgtgggacattattttgcctatcacagcaaggttacatggctgcctgactcttggaaagaaatcctctgattctcaaatccaatcaaatattatgtaaaaacagcacttccaatataatcttcccatcttttcaggatacatgtgatgagtttagaagccaaatgaaaaatggaaaacttatctgcactcgagaaagtgaccctgtccggggtccagatggcaagacacatggcaataagtgtactatgtgtaaggaaaaactgtgagtatgtttcaaaatgagcttttgactgtgagtcttaaagtacaataatcatttcttaccagtttgggaaaatgacaattgttttagaagcagatctggtaattaatgaggcgtttgttcactttgattgaaatgtttcattgttttccccccagggaaagggaagcagctgaaaaaaaaaagaaagaggatgaagacaggagcaatacaggagaaaggagcaatacaggagaaaggagcaatgacaaagaggtaatagatgttagacacgctaatacctgaattcagttagttcattgtatggtatatttattcaacaaatatttgtgaaatgctgactctgtcccaatcattggtgatataacggtaaacaatgaagtcatggccagatcttgaaaataaatagcatgctcttcagttccccaggagtgactctgatgcaattgtagaaccagtgacaactgtcaaattattgtagttagccagtgaatttcatttttgaattttttctttcctttgagacagggtcttgctgttgctcaggatggtctcgaactcctgagctcaagcaatttgccggagctcaagtctcagcctcccaaagtgctgggattacatgagccatcgcactctgctgtttctgaattttttaaacaaataaatatcaagcaatcagatgccaaaaattacaaagaaaatcagtatcaaaaatttggagtttgaggccaggcacggtggctcaggcctataatcccagcactttgagaagctgaggcgggcagatcacgaggtcaggaaatcgagaccatcctggctagcacggtgaaaccccgtctctactaaaagtacaaaaaaattagccgggcacgatggcgggcgcctatagtcccagctacttaggaggctgaggcaggagaatggcgtgaacctgggaggcgagcttgcagtgagctgagatagcgccactgcacaccagcctgggcgacagagcaagactctgtctcaaaaaaaaaaaaaaaaaaaaaaaaggaaaagaaaaagaagaaaagttggagtttgagataataaattcttgatctggcatttcattgccacctagtgttcagttttggtccttataaatagtattcaacactacaacaccaacacgtagaatattacacagcacaatcttttaatgcaccgacggactccaattagaaaagataaatagcgaaatttccctgggaagatttttcctattagtagggcaacttttcattaagcctgtagtggctaaagaaaatgaaaccttattagtaattatcattatttaaaaatattttgggaatatctgttgtgtgttacggtagatgaaatggctttgagaaaataaccttattgcacaacttcacatgatctctaaaggacatctatttaaaatcttggtaaatttttaattttatgatccttatttaaaaaattttgaacagaaaaatgctatatatatttatatatagaaaatactgagtaaaaaaaaaataaagaaaatatggaggcagggtgtggtaactcactcttttaatcccagtgttttgggagaccagagcaagagaattgcttgagcccaggagttcaaaaccagcctgggcaacatagtgagaccctgtctctacaaaataataataattaaaaaaaaaatagccaggcatgatggtgcacacctgtaactctagctacttgggaggttaaggtgagaggatcacttgagcccatgaattcaaggcagcagtgagctatgatagcattattgcactccagcctgggcaacagagtaagaacttgtcttaaaaaaaaaataaaaatattgagattgtgtgtgagtgtgtgtgtgtgtgtgtgtgtgtcaaatagagaactaccgttttatctacacatgcaacgttaaataaggtttaccaataattatatttaaacctccccaaaatctattcagtggtgatattgttccaattttttagaattcaggaaaacttaagagaagtgaactacacataattaatgaatagaagagccaaagactgaattggagggttgcaatacagtcagaggagtgctattataaaaatatgtggtattgtgagcacctggaaaagaaaatacttccagtaatttgtgaaaatggtggttgtgagacatttaatatgttgataggtatttaattccagagaattttttaaaatgctgcaattaaaagtaaggcctggaattgtttgtttacggacatcacaatttaaagaaagaagggtaagtacaatctaggttactgtgttattttttaaggtaatgtttccatatcaccactttggctctttgggcttacaggaagttccagagacttctgtagctctctgaagattttagtacattctagatcctatctgtagactcggttctattggcttctttttatgatcattatgtttcaatgaacacaaaaggcagctccatatacttatccacacataggagttttattctctgacttgtaaaagcaaataagtaaaaaggtataacctggtggttagcaggtttctgatcacaaattcaacactgactttatttaatagctctgagatttatttttctcatctgtagaatggggatgatgcgtatttatcttataagatttttacaaccactaaaggaaatctattaaaccacacgataaagcacctcgcacaataatttttttttttttttttttggtaaattgtggttgagaaatgaagctgaaagtttacgtgtttttatgttttgccagaagagtgtcgtttagttcattccagggcttttgaagtagacttatgtgtggaatactgagtgcactcatagggagacaggtttggggctctgcctcatagaaaatggtattaaaatgagatgggacagggcactgggggctcatgcctgtaatctcagtgctttgggaggccaatgcgagaggatcacttgaggccaagagtttaagatcagcccaggcaacacagagagactcccgtctctacaaaaaattttaaaatattagccaggcatggtggcacatgcctgtagttctagctacttagcagactgaggtgggagtgttgcttgagcccaggagttggaggctgcaatgagctatgattgcaccactgcactccaggctggacaacagaacaagattatatatatataacaagattatattatatacataacaagattatatatatatatagatagatataatatattatatatatagatatatattatctatctatctatatatataatcttgttatatatatgtaatcttgttctgttgtccagcctggagtgcagtggtgcaatatatgtgtatatatatatatatatatatatatatataatcttgcttgcaatatatatatatatagtttttaaaggaatgggctgctttagtgaaattccattagggcaagacaatcatttgatagagatgcaatggagaagattcaaacttcttaggtagagattgaataaagtgcctttaaaacatatttcttctctgaaatttcacttctctgtttttttcctgtgttatgagtttatatctaatcggtcaatcatgttatcaggtttgaaagattataccatgacagtaacaactttttctgctactgttggtaggatctgtgtcgtgaatttcgaagcatgcagagaaatggaaagcttatctgcaccagagaaaataaccctgttcgaggcccatatggcaagatgcacatcaataaatgtgctatgtgtcagagcatcttgtacgtaaaaaggtttatcaataaatttgatagttgtgcctgtttgctagaaattagtttttgttacttttaaaacctaagatacttggcatcacacattaatgatatgatacctcctctcttttgaagagacaatttccaaagcactttcacaataatttttttattttgatgttgaaaagcataatcatacccagaacctctggggtaggaagaggtagcttatcagctaggtaacctgggacaggccagttcattgtgcagagcctcagttcctcctgagggaaaaatgactgaacaatatagttttgtggagattcaaggacattttaaaaatgtttatgaacttcaaagcattatacctacgtaaataacattcaaggacattttaaaaatgtttatgaacttcaaagcattatacctatgtaaataacaatcaatattgtgagtttggttataatttaattggaaaagtaaaaatgagaattatagcaactttctagatgagagtcttaagtctattatattaaggaaaaataaagaccttttaggatcatatttttctggataagagtatagtaacagcatctatgaaagttgatagcattgccacaaatatattatcatgaaaattccaacaatcagaactgattagcttataaaaatattcttagatttttttttaggtataagaaaacagtgttagatttgctaatgtttagaatcgcagaaatacttggttaaagacaattcagtaacaacccttgaaaaattaccctatctttttttttaattattctgcagtgatcgagaagctaatgaaagaaaaaagaaagatgaagagaaatcaagtagcaagccctcaaataatgcaaaggttatttattaaaggataccaaaataaccattttacttttcaccttcagaattttgcattcttctccagctttgggaataataaatatattaatatcttaacttcaaagaaaattgatttttcttgtcttgtcactttgtatcataacaagatttttgggggtttgggggtttgataccttttttgattttgtagaaaattctctgagagtgtgtgtctattattgttttagtctttcctccttgggagaaatatggattgaaaagtagagggtaacattctttaccaactcaactgcatgcttatctttgacactttaagtgtgaaacaaggctttgggtagcttgctgatgtttattttaataattcaggcaatataaaccaatctaagtctcccttttagcttcccttctgttttcaccagatgattctatcagcattaaagaatcagcagtgtgcatgcgtgatgctttcttttctatttatttttcaagactacaaagaagacatctttattcaattctttaacagtaacaatcacatttgtagagttagatccctgagcaattgtccttttgtcaaaagcatgcttcatggggaaaatggggaaaggaaggatggatgaacgggagaaaaaaacaggaaggtctatcaagtgttgttttatgtttccctacatttttcaggaccagtgcagacaggttcagaatgaagcggaggatgcaaaatttagacaacctgggcgttccttggcctctgttgccaggatgagtacagtgagtctgagcccagagctaagagaggacttcctaaaaccaagtttgaagaaatcactaagccaaaaagggacaaagccatgttcactttccctttctcattttctaggatgagtgcagtgaatttcgaaactatataaggaacaatgaactcatctgccctagagagaatgacccagtgcacggtgctgatggaaagttctatacaaacaagtgctacatgtgcagagctgtcttgtgagtaagaggattctgctccccctgtagctagcaggggaactgcatttttagaaactgctgcttgaataagtttgtatatttatgaaccccatgggattttaaaaataagtctttagaattaaagccctcaaattgagaacacctgatatagtagaaagaacactgagacttggagacaaaaattccgtatgtaagttctaaccaagatgtatactagctgtgtcattaaacaatttgttttcaactttcagcatcataattagaagaaattcttcaagatctttattccacaactagatcagactcaattgagatgcctttaaaaataaatacaaatttttacaccccaaaatatattatatcagaatcttggggctatggaatttgttttttagaaagagtttctaggttattctaaaaagcaaatagaaatggagaccaatggacattataaatgatgatgatgatgatgatgatgatggttgtaaagcacactgtgaacacaacaataatcaaatttctttagcttttcatagtgagtgttctttgagggggaattaccaaatactactcccattttatgttctagaaaatcaaaacttcagaacattaacccacttgtcatctgttaagtgatgaaagttctaaaccagctcctaattgtgtacttttcttaatatgtcatcctggtcactatctcaatgttctgtgattctgttttatttatttatttatttatttatttatttatttattgagacagagtctcactctgttgcccaggttggagtgcaatggtgcgatctcagttcactgcaacctctgcctcctgggttcaagagattctcctgcctcagcctttcgagtagctgggatgataggtgtgcaccaccatgcccagctaatttttgtatttttagtagagatggagtttcaccatgttggccaggctggtctccaactcctgacctcaggtgattcgccagcctgggccttccaaaatgctggggttacaggcgtgaactaccatgcctggcctctgtcacattttatgaggattgcttaacttctcactggaagttataggaactgtttcttattttaaattattttttattttcttctctagtctaacagaagctttggaaagggcaaagcttcaagaaaagccatcccatgttagagcttctcaagaggaagacagcccagactctttcagttctctggtaaggaggactatttctgaaaagctacttatcaatttaattttcttgattttttttgacctgttgctatttgctattttcatagaagggtctgaggtttgctgattcagtgcagcctgaagtcctttgaggactatatgcagtttatgcttggtgagagttctgggatttctcattagtgccttatcctgtgccctttgagattatttgccctgcttcaatttagtacattttgaaatttacaatgtgtgtgtggaataattgtgactaatttcacatatggtaaaatacatagtcaggtgcctcaattctgtaattttagcatacctcttggtattcttagcttcctcacctagcttccgcttatttccctcctccaaccaaatctgtttctacatcctggaactctatgccaagcatgctcacaggtataaatttccgccaattagcaattcgcagtagctaaagcttcttgtttaaaatataggctgagctgggtgcggtggctcatgactataaccccagcactgtgggaggccaagcgggcagatcccttggccccaggagtttgagaccactgtgggcaacatggtgaaactctgtctctagaaaacttacaaaaaaaaaaattagctgggcatggtgccttgtgcctgtagttccagctacttgggaggctgaggtggaaggatggcttgagcccaggtggtcgaggctaccatgagccgtgatagtgccactgcactccagcctgggtgacagaacaggaccccacccttctctcttcctctctctcgtagctgatggtcaccagtaatgtaagtattagtctacattagcatgataagatttactttgtagagccttcacaagatgaggagtagaaaggagcaaagaggatatgagaacatcaagcagattctcagattgggccctaaaaggaagattttagcacctaggctgatgacccagaaaaggagaaagaaaggtatcaaacatgaattttacttcattattcacccaattcaactagggtaatagatgagttacatacatgaaatatgcattaattacatatttctggtaaattgagctccacatcagggatccgaaggcatggagaagagctattaacaaaatatgtttttttttttaaatactaaagtataatagttacatagaagaaatgtacatatgcaagtaaatagctaaatgaaatatcacaaagtgatcatacctgtgatagcagcaagaaataagatatcatcagcagaccagtagccccctcattctccctcataagctacagtctgctttggagctatttcttttggagaaataaatctttcattattcatttttttcatcaagccggcagtccttttgtcagtctacaaatacttactgagcacctgctatttttccaggcattatttatttacttatttatttatttatttattttctgagagggagtcttgctctttcgcccaggccggagtgcagtggcgcgatctcggctcactgcaagctccgcctccgcagttcacgccattctcctgcctcagcctcccgagtagctgggactacaggtgcccgccaccacgctaattttttgtatttttagtagagacggggtttcactgtgttagccaggatggtctcgatctcctgacctcgtgatccgcccgcctcggcctcccaaagtgctgggattataggcgtgagccaccgcgcccggcctccaggcattatttacgtatgctaggttaatagccataaacaaaacagatagcgctcactacatgaagttcatagtatattggaaaagcagatacaataaataatatgaataaatgaaaaaataatgccaaggaggaaataaggcagaaaaaaatggtacagtaaattctgagatggggaggggaggggatacaattttaagtgacatatacagagaataactctctcagaaggtgacatttacatcaagacctaagggtattgattctgtaggtatatctgaggagaacatattccagagagaaaacatagcaaagcgtttagtgttgttgaaatagagctacctcagctgtagaagaggaagtcaggaagctaatgggaggccatgttataaagggttttgtagacaattttaaagactttgcctaatattctgaatgaaggttctgagaagactaatatgatttgatttatgttttaaaggaataactttgcctgatacctattgaaaacactgcagggaggcaagggctggacagctagttagctattgcagttgtctacagaagagatgatgctggtttataccatgttagtagtggtggagctcttgagaggtggtaagatactgggtctgttttgaaggtggagtcaacagaattttttgagttgatgtactatgttaaaaaaatgaagagagagagagagtaaggaacattgtagtaccttttgaacattatagcaccagttacctgaaaaaatacaattgatatacctggttgagtctaacctttgccagggtttcatataagttacttatctaaattctctgtgaatatttgtaccaatatggaactcaccacaatctaaagcaattcatttaggtgtttgaatcatttatcaaaacaaaggaacaagtacacaaatactgcttgactcctccaaaatgagtattagcagatataatactattaaattttctatgtcgtctttgtatctgttttcagtaagtagttttcaaggaatttattaatttcatctatgttgtcaaattttttggcatgtagttatttataacatttctaaattattcttttagtatctacagctcatatagtgatgacctgttatctctaatattggtaatatatgttttcttgactctggtcaattttgcaaatagcttatcaacttattcttttcaagcagctaacttttggctttgttctattttctatttcatcaatttctcctctttattacttattttaaaaaattttctatgctcatcattggctagctccttcagatgaaagtttagatcatccattttagaacctctttcttttctaatatatgtattaaagttataattttcccccaaattactgctttggctgctgggatttgataggttgtattttctttgtgattcatttcatttctaaattctatgattactttgagtcatgagttatttagaactcatccttttacttataaatatatttcttataaatattatatagttgattctttttgttcttccagttggataatttctgtcttttaactggagtacttacttcttaatattctatgtaattactgataccattggatttaagtcttccatcatcctatttgcttttgaatactttttttatattttggctctactttttgcctttttaaaaaactaatcaaggtcttggaattatcattaattattaaactcatacttccaaaacactacgaagagcaaatatgtttcttcttttacatggaaaatatacatatgttttttaccgtggaaaacatactaacaactaaaatttactgaacacttagtatgtgacagatactgcgttaagcatgaacttacatgcattatctgttttaatcccttcaagcagtaatataaagtaaggactactattatttatgattttctggtgagaaaattaaggctgagaaaagttagataagttacttctctagttagtggtagagctaggactcaaaaactcatgcaatttatgagtatttacctctgagtctttcctttctaggctgtatataaccaataccttcacttaaaaaggcgaatattatttctggcatgcctatcattctagatttcttcttatgggcaggtaattgtgtcacatattataaaggatatttgctttcttattgctttgctactccttgtttctttgtttacttaccttgtcaagttaaaaattgccaaatacaggaagttaaatccacctcatatggtgcaaaatcaatctttgagtttgaataacatatatcacactccttttacaaccatttattcaagttggattaaggaactcaagaggttttcttaagcccacccctcttcttgaatgccataaagtacgtctgctttattttttgcttcttcaggattctgagatgtgcaaagactaccgagtattgcccaggataggctatctttgtccaaaggatttaaagcctgtctgtggtgacgatggccaaacctacaacaatccttgcatgctctgtcatgaaaacctgtaagtattcaagttgccccatcatatcttccagtttagaatttctcagctagagtgttaacccatagtaatgcactgatataaattcgaaatgtgttgcaagtaatttattcatcatagaagttaaaaattcaaaatttgtgaattatctttcttatatgtaaaatagaaaagatttaaggctattcctttggtaaattgcttacctacattgttatatgctctactgagaagaagagaaagaaaaaaaatagtggagttacaattgcagaatattggtataatcctgtgtacagtcatgggagtgtgaacatcattcattctgtgtgtagtggagaaaaacagtagagaactactgctatataacccagcccagtgtattatatctaaaaggtcctgtgactataatatagctacatattccccatttctttaaccctgcctcttgaaagaagtagtaccttgcaaaaggaaagtgcactgtacaaaagaaagcccaataaataaatagctgatctgatgtctgctctcatatttatgactatctgggtatctggaggcatattatttcatgattctgaatctcactcccttcattggagaaatggagacagttctctgagctgctgacttcacagatgattgaaaagaactgctagatatgaaaatatttttatcttaagttaaatacaagggattcattttaatactcttattattggctttagttttgttgttgctactctgctcaaatgctgtctgaattttaaaataatttttaaataatttccccttgtctctatatgcatcccttcccttctcctggccaccatttggccagctttcaaggagagaatggtgacaaaccttggttttcccatctcacctgaccccagacctacattgtagtactactttatccttcttaggacactataagcaaggcccagaacatgaggataggactccctgatatcctgctttttaatcctggaaaaatatttaaaagaattcatcttttaggtaaggtatggtagatataggtaaggtatggtaggtatggtatggtaggtatacttcatctataggtaaggtatggtagataaggcactttcagatttcagtcactgaatccctgcaacctgctatggaatgttattatctttattgttcagaaggttaatctgaggctcagagagtccaggtaatttgtttaaagttacaaagctagggagtaaatcagacaggattttgaccttattgatttggatctaaatacagttttcttttttctccactaaactactgttatttctaagacagtagttccttaaattgtcaaagaccaaagattccttagataatatgaagaaagttactcctttcaagaaaatttaaaaaaatgtgatatgaacaatccacacacataatattcataggctcgtggagtacttgaaattcatctgtagaccttataggtttttaagaattaagaaattcccgtttaaggaataaatataactgtccaggaattccattactatatataccttatctccagcccagaaatcctttaattcctctttcttataaatgtcagggcctaaattatccatataattttcttcaacattttcatcaactttatagacccataacaagtgatctttttaagggaaatttagcataagaaactgaaatgatgaaaatattaagaaagcatacctcaggatagtttgtttagctaagggagctgggagtagatattataaagaaggatcaggttatctgaggcaaaaggaaacaatattcatttctaaggttttagagaaagttatatgagagcaatttcaatttaagcaatgctaaaagacaaattgcactgccttgatagataatgagtttcccattaccagaggcaaacaagtacatgtctgattcataatatttctccagtgagaggcattaaaattcgtactccttaagattcattccaactacaaaactcagactgtatcatttgtgtcttgttatatttttccccaggtcgtattattggtactcaatatagtcaaacatctctctgggatctgattctcgcatctataaatgcgagatcacctttagaccagatgatctccaagtttatttccaacattctctatgaatatatcagtgaatgaaatgtgacaggataggtccagggctaggggatgaatctggagcagaagagagacagcctggatgatacctactgaaaatgaaagtagttgaatgcagatcccagatcctccctctaaatacagtttggtttgatacccaagtgtcctgcatgttggtccttatttattttcttattataactgagaacttcctcgttgttgaagcatcctctgatctgttttaggatacgccaaacaaatacacacatccgcagtacagggaagtgtgaggagagcagcaccccaggaaccaccgcagccagcatgcccccgtctgtaagtacataagtagactggcctccatggttacgttgtgaggagcactgggttctggttttgttctcttccactgagtaatggacatttatctggccagagaggtgacactaggtcaggtgacccagaattggtcacattttcactataaggataattaagtaattactgaaataagtggaaataatgtagcagttaatattttaaaatatttaatgggtccattaatagattgtttatgagcaatgtctccaacataacgttacttatttcaggataggttttgttttctgtgatctataataaaaagatgagacagagaccaagagaggtcagagagtgacagagttggatttaaacaatttctatataatgcccatattcttgaactttacattgtattaaattatttcccagcattaaaagttgaccaatttcatttaaactcctgaaaggatgtttcaacgtacgttactgaatttaacattctccctggtggctatttgaatagtcatcctcagggagatggtatgtaacatgaaaaaataggaattttcaaataagagaaaaatgttttgttatgggataaatgtgagtgcaaatttcagtttttgctgtgcttatacgagactcttggcaaatcacataaatactctaaaccaaatatcccaatgacctatgaaaatattcaataaatactgacttttatgcatttggcacagtgtctgaaaaattaatgtcactattattattatgcctaagaccaaagtatgaatttagaaaaagagtcgaaagtatgaactttttctaacagaccatggtatagtgcttatttatttttaataaattattgtcaattgtagtttgataaaattatgtgtaataaacaatattttaatgcattatatttaataaagctatttattattattcacattctctttgctccattccactgccacaaaaagctttggcaataatggaatcagaaactattcagccagatttgaaaatcttagtcaagcaatactagataaggccatattaaaaacgtctgcccaacaatctttgttaagggaaattgtgaaaacaagaatcataagctagagtcttcctagtttcacattagcagctgaaagcaatgtagaggaagccgaggactggaatgaaatcagaagaccttatctttctcatgtgggcttgacaaagttatttaccacccatttaaagacctctactctggttctttaccaggatccataaaaagggagtttaaactttaactctagtccctatattgtgtttgctgtcttattatatagaaattcttagtagttggctcccattttattgacagaatatactgttccccgtcctcacaaactcacctttacatatatctcaggagagaaaggtcaaaatagctttatgccagtccaatagaccacaccttagaccacacttccctgtaactctcatcctgtgtaggcaacttggaaaaactactttataaagaacttgccataggtttatactgataagatgtatttctttaaataatgaagaatgttgccggggcttatcacacagggtgttgtaaaagagtgccgttcttccatttctgagtgtatgctttccactggagttgcatagcaggcattaataatgacagaaattattatgctgttagttaatgaaacaaaaattaacgtgtcctggacacaggtggaacttcagtccctgtgtaagattcaagtgtgtgatctcgggtacagttggtacagactggtcatggcctagcattgttctgatgactagtaaggatgtatatattggggtggaagcctgttgtttgaactttatttctgggtggaagtttctctaagaaagtctggaatctcaagaatagaataagagtccaggcagtcacaagtttcttggaaaagtaggttgagtatcttggaacccactcttttgttactaagtacataatactttctattatgcaataactattatacagtaactactaagatttacataggtctttagagttttatgtacattttctgtttgtactcttacaatagtcatttgattagggcagggatcactgtctttcccattttacagaaaatcatcctgggggaaactgtcacttattaaagtcccttagtataatatcttgggttcaatgtagatatcctgacttcaattccagtgctctactaccattccttagttgtcaggagattctgaaaactaagtgctgttgagaaggcattatctcaattaaaagatgatttgctatattggaaggaaatttctatattaagaaaaatacaaatattattttgtgagataaatgactattcttatctatatctgtcaatttcatgacagattttcaaggattcaggaaaaccga

SEQ ID NO: 12 - DNA, 1037 bp, Homo sapiens
  misc_feature (1)..(1): n = a, c, g or t/u
nggcttnccataagtttgttggagattnaaaaaanaaattttggaaangtntttaaatggcattcagcatttcagatcagaaccgagctgttttcaacaaaaaaccctccggagtnaanggcanaagngantttggttggaatacagtctgttgnccccaggggnactagtaaaatgcccgttatgtaaaaaaacttcctgaggccntcgccccnttaagattatttttaaanattcattnaatgtttaaaggnttgtttncaaaaaaatggatgagtccggccaaggaaagccagctgattacattctagtggtcnnccnccnanctatagtagaaaaacctttccatattncagnaaggaattcaagttatgccctaacatgaccaggggcaagttacttaaacttcttctgaaggctcaatttccntcatctgcaaaatggtgctaatgacaccctacttcttttttatcagcatgaaggctgcatgtaatcaaaggccagaatttaagcccatgttcatatttgtggcccccctcaacacggtagtacatactaggtgctcaatatttgttgtatcaaactggataaaatgtgaaagtgattttaaattgctacttctttgcagaatgcaggatctatggatttctttgattaagatgtagctataaatatacagcccacatttctgcaatatctctgggttctagcatctaacctacccatcttctcttctaggacgaatgacaggaagattgttgaaagccatgagggaaaaaataaaccccagttctgaatcacctaccttcaccatctgtatatacaaagaattcttcggagcttgtcttatttgctatagaaaacaatacagagcttttgggaatggaatcactgattttcagtcttttccatttctttcctcctagaatctgtgatctgagggtataaagacatttccaccaagtttgagccctcaaaatgtcctgattacaatgctgtctgtccaactgcctgttcaataaaagtaaactcagcag

SEQ ID NO: 13 - protein, 19 aa, Artificial Sequence
  Description of Artificial Sequence Peptide
CEESSTPGTTAASMPPSDE

SEQ ID NO: 14 - protein, 19 aa, Artificial Sequence
  Description of Artificial Sequence Peptide
EYRKLVRNGKLACTRENDP

(19) United States
(12) Patent Application Publication
(10) Pub. No.: US 2003/0190637 A1
(43) Pub. Date: Oct. 9, 2003

(54) Mutations in spink5 responsible for netherton's syndrome and atopic diseases

(76) Inventors: Alain Hovnanian, Oxford (GB); Stephane Chavanas, Oxford (GB); William Cookson, Headington (GB); Miriam Moffatt, Oxford (GB); Andrew Walley, Oxford (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/220,510

(22) PCT Filed: Mar. 2, 2001
(86) PCT No.: PCT/GB01/00897

(30) Foreign Application Priority Data
Mar. 2, 2000 (GB) 0005098.9
Mar. 3, 2000 (GB) 0005229.0

(51) Int. Cl. C12Q 1/68

(52) U.S. Cl. 435/6

(57) ABSTRACT

The invention relates to the identification of the SPINK5 gene as the gene which is mutated in the autosomal recessive genetic skin condition Netherton's Syndrome and as a susceptibility gene for atopic disease in general. Genetic screens, therapeutic products and Nucleic acids and proteins corresponding to mutant versions of the SPINK5 cDNA and expression product are all described.

The present invention relates to the identification of the SPINK5 gene as both the gene which is mutated in Netherton's syndrome, a rare, autosomal recessive genetic skin condition, and a susceptibility gene for atopic disease in general, and to genetic screens and therapeutic agents arising from this finding.

Netherton syndrome (which may be abbreviated herein to NS) is characterised by the association of abnormal keratinization, trichorrexis invaginata and atopic manifestations. Life-threatening complications such as microbial infections and hypernatraemic dehydration result in high postnatal mortality in infancy.

Using linkage analysis and homozygosity mapping, the present inventors have mapped the NS gene locus to the interval D5S463-D5S2013 on chromosome 5q31-33, 8 Mb distal to the cytokine gene cluster (CGC) (Frazer et al. 1997). This region has been associated with high IgE levels, atopy, asthma and autosomal dominant familial eosinophilia (Meyers et al. 1994; Cookson, 1999; Rioux et al. 1998).

By searching for mutations in plausible candidate genes/ESTs within the critical D5S463-D5S2013 interval the present inventors have now identified a gene designated SPINK5, encoding the serine protease inhibitor Lympho-Epithelial Kazal-type related inhibitor (LEKTI)(Mägert et al, 1999) as the NS gene. In an initial study a total of 12 different SPINK5 mutations have been identified in 13 affected families including nonsense, frameshift and splice site mutations.

In addition to the principle manifestations of congenital ichthyosis and trichorrhexis invaginata, atopic symptoms are a universal accompaniment of Netherton syndrome. NS patients may have personal and family histories of hay-fever and asthma, high elevations of the total serum IgE, and recurrent urticaria or facial angioderma. The identification of SPINK5 as the Netherton disease gene has allowed the present inventors to further test for genetic linkage and association of markers in and around the SPINK5 gene with manifestations of atopy.

As will be described hereinbelow, the inventors have identified polymorphisms in and around the SPINK5 gene which show association with atopy and asthma in children with atopic dermatitis. The genetic association shows parent of origin effects. These results identify a novel pathway for the development of allergic disease.

Therefore, in accordance with a first aspect of the invention there is provided a method of determining whether an individual is susceptible or predisposed to atopic disease which comprises screening the genome of the individual for the presence or absence of one or more polymorphic variants of the SPINK5 gene.

References to the ‘SPINK5’ herein should be understood to refer to the human gene encoding the LEKTI serine protease inhibitor (this protein is described by Mägert et al. 1999). The protein product encoded by the SPINK5 gene, including that encoded by variant SPINK5 alleles may be referred to herein as LEKTI, LEKTI protein or the SPINK5 gene product. The SPINK5 gene was originally designated “SPINK3” but the gene nomenclature was changed at the request of the HUGO Nomenclature Committee.

The method of the invention will preferably comprise screening the genome of the individual for one or more polymorphic variants of the SPINK5 gene which have previously been demonstrated to show statistically significant association with susceptibility to atopic disease, for example in a family or population-based genetic association study.

The polymorphic variants will commonly be single nucleotide polymorphisms (SNPs). SNPs within the coding region of the SPINK5 gene may result in a change in the amino acid sequence of the encoded protein or may be silent. SNPs in intronic regions of the gene may result in alternative splicing events.

Most preferably, the method of the invention will comprise screening for one of the specific polymorphic variants of the SPINK5 gene identified herein as risk factors for atopic disease, specifically 1103 A→G or 1258 A→G.

Atopic diseases such as asthma, eczema and hay fever are complex, multifactorial diseases and a given individual's total genetic risk for developing atopic disease is likely to result from the presence of a combination of gene polymorphisms. It is therefore within the scope of the invention to perform screens for the presence or absence of one or more polymorphic variants of the SPINK5 gene in conjunction with screens (in the same individual) for other polymorphisms associated with atopic disease, for example as part of a panel of screens.

The further polymorphisms associated with atopic disease need not necessarily all be single nucleotide polymorphisms but might include other types of polymorphic variation such as, for example, variable number tandem repeats. The further polymorphisms will preferably be ones for which a statistically significant association with atopic disease has been demonstrated, for example in a family or population-based association study. However, it will be appreciated that the panel might also include screens for polymorphic variants which are either in linkage disequilibrium with or in close physical proximity to a marker shown to be associated with atopic disease but which have not formally been shown to be associated with atopic disease.

As would be readily apparent to persons skilled in the art of human genetics, “linkage disequilibrium” occurs between a marker polymorphism (e.g. a DNA polymorphism which is ‘silent’) and a functional polymorphism (i.e. genetic variation which affects phenotype or which contributes to a genetically determined trait) if the marker is situated in close proximity to the functional polymorphism. Due to the close physical proximity, many generations may be required for alleles of the marker polymorphism and the functional polymorphism to be separated by recombination. As a result they will be present together on the same haplotype at higher frequency than expected, even in very distantly related people. As used herein the term “close physical proximity” means that the two markers/alleles in question are close enough for linkage disequilibrium to be likely to arise.

The term “atopic disease” is to be given its normal meaning within the art. Included within the spectrum of atopic (allergic) diseases are asthma, infantile eczema and hay fever. The atopic state is characterised by elevation of the total serum Immunoglobulin E (IgE) and exuberant IgE responses to common respirable proteins (allergens) such as house dust mite and grass pollens. Atopy is influenced by genetic and environmental factors, and loci influencing atopy have been localised to a number of chromosomal regions, including chromosome 5.

As discussed in detail in the accompanying examples, the association of allele 1 of Asn368Ser (1103 A→G) and allele 1 of Glu420Lys (1258 A→G) with atopic disease shows strong parent of origin effects; the relative risk of the maternal allele being approximately 4 compared to the same allele when paternally inherited. Accordingly, in performing the genetic screens described herein it may be necessary to take account of the parent of origin effect, for example by also screening the parental generation.

In accordance with the invention, the process of “screening for the presence or absence of a polymorphic variant” of a given gene may comprise screening for the presence or absence in the genome of the subject of both the common allele and the variant allele or may comprise screening for the presence or absence of either individual allele, it generally being possible to draw conclusions about the genotype of an individual at a polymorphic locus having two alternative allelic forms just by screening for one or other of the specific alleles.

The step of determining screening for the presence or absence of specific alleles, also referred to herein as ‘genotyping’, can be carried out using any suitable methodology known in the art and it is to be understood that the invention is not limited by the precise technique used to perform such genotyping.

Known techniques for the scoring of single nucleotide polymorphisms (see review by Schafer and Hawkins, 1998) include mass spectrometry, particularly matrix-assisted laser desorption/ionization time-of-flight mass spectrometry (MALDI-TOF-MS, Roskey et al. 1996), single nucleotide primer extension (Shumaker et al. 1996; Pastinen et al. 1997) and DNA chips or microarrays (Underhill et al. 1996; Gilles et al. 1999). The use of DNA chips or microarrays could enable simultaneous genotyping at many different polymorphic loci in a single individual or the simultaneous genotyping of a single polymorphic locus in multiple individuals.

In addition to the above, SNPs are commonly scored using PCR-based techniques, such as PCR-SSP using allele-specific primers (described by Bunce, 1995). This method generally involves performing DNA amplification reactions using genomic DNA as the template and two different primer pairs, the first primer pair comprising an allele-specific primer which under appropriate conditions is capable of hybridising selectively to the wild type allele and a non allele-specific primer which binds to a complementary sequence elsewhere within the gene in question, the second primer pair comprising an allele-specific primer which under appropriate conditions is capable of hybridising selectively to the variant allele and the same non allele-specific primer.

If the SNP results in the abolition or creation of a restriction site then genotyping can be carrying out by performing PCR using non-allele specific primers spanning the polymorphic site and digesting the resultant PCR product using the appropriate restriction enzyme (also known as PCR-RFLP). Restriction fragment length polymorphisms, including those resulting from the presence of a single nucleotide polymorphism, may also be scored by digesting genomic DNA with an appropriate enzyme then performing a Southern blot using a labelled probe corresponding to the polymorphic region (see Molecular Cloning: A Laboratory Manual, Sambrook, Fritsch and Maniatis, Cold Spring Harbor Laboratory, Cold Spring Harbor, N.Y.).

Polymorphic variants may also be scored by sequencing regions of the genome. Typically, a region of the genomic DNA including the polymorphic locus will first be amplified, for example using PCR, in order to provide a template for sequencing.

The known techniques for scoring polymorphisms are of general applicability and it would therefore be readily apparent to persons skilled in the art that known techniques could be adapted for the scoring of any of the SPINK5 single nucleotide polymorphisms mentioned herein.

As would be readily apparent to those skilled in the art, genotyping is generally carried out on genomic DNA prepared from a suitable tissue sample obtained from the subject under test. Most commonly, genomic DNA is prepared from a sample of whole blood, according to standard procedures which are well known in the art.

Where the SNP results in an amino acid substitution in the protein product encoded by the SPINK5 gene, screening for susceptibility or predisposition to atopic disease could alternatively be accomplished by screening for the presence of the variant protein rather than screening for changes at the DNA level. Accordingly, the invention further provides a method of determining whether an individual is susceptible or predisposed to atopic disease which comprises screening a tissue sample from the individual for the expression of a variant LEKTI protein. Most preferably, the method of the invention will comprise screening for one of the following single amino acid substitutions:

368 Asn to Ser or 420 Glu to Lys.

The methods described herein provide simple and straightforward screens which may be used to identify ‘at risk’ individuals who may be more susceptible to the development of atopic disease by virtue of their genetic make-up. The ability to identify ‘at risk’ individuals using these genetic screens may allow early intervention with preventative treatment strategies or may allow ‘at risk’ individuals to minimise their exposure to environmental risk factors.

In a further important aspect the invention provides a number of screening methods for use in determining the carrier status of an individual for Netherton's syndrome or in diagnosing Netherton's syndrome in a patient.

The first of such methods is a genetic screen which comprises screening the genome of the individual or patient for the presence of loss-of-function mutations in the SPINK5 gene.

As described herein, the present inventors have identified 25 different types of mutation in 34 affected NS families, demonstrating that defective expression of SPINK5 is the basic genetic defect in NS.

The loss-of-function mutation may be a nonsense mutation (e.g. a single nucleotide change leading to the incorporation of a premature translation termination codon), a frameshift mutation (e.g. an insertion or deletion of one or several nucleotides which may again lead to premature termination) or a splice mutation leading to the production of an abnormally spliced mRNA. It may also be a mutation occurring in a regulatory region of the SPINK5 gene, e.g. the promoter region, leading to an abolition or reduction in the levels of expression of the LEKTI protein.

In a preferred embodiment, the method will comprise screening for one or more of the specific loss-of-function mutations identified by the inventors in NS families, namely 81 G→A, 2258insG, 153delT, 238insG, 283−2A→T, 2468insA, 720insT, 1086delAT, 1888−1G→A, 2313G→A, 2369 C→T(R790X), 2468insA, 1038insG(A)₄, 1111C→T(R371X), 81+2T→A, 2459delA, 2259insA, 2240+1G→A, 81+5G→A, 1608−1G→A, 2041delAG, 649C→T(R217X), 628C→T(R210X), 56G→A or 377delAT.

A number of different techniques may be used to screen for the presence of SPINK5 loss-of-function mutations in accordance with the invention. For example, insertions, deletions and single base substitution mutations may be identified by sequencing all or a part of the SPINK5 gene, possibly following PCR amplification using SPINK5-specific primers on genomic DNA. In addition, any of the techniques described above for the scoring of single nucleotide polymorphisms may be used. Genetic screening for NS carriers or for diagnosis of NS in patients is preferably carried out on genomic DNA extracted from peripheral whole blood. An important aspect of the invention, described below, is pre-natal screening for NS mutations which is preferably carried out on DNA extracted from material obtained by chorionic villus sampling or from amniotic fluid. Accordingly, references herein to screens being carried out on ‘individuals’ and ‘patients’ are also to be interpreted as encompassing pre-natal screening carried out on pregnant mothers with the intention of diagnosing NS in the unborn child.

In addition to the genetic screen described above, the invention also provides for screens for use in determining the carrier status of an individual or in diagnosing Netherton's syndrome which are carried out at the protein level. The first of these screens involves evaluating LEKTI protein expression in a sample, for example a skin biopsy, taken from the individual or patient. Advantageously, this screen may comprise simply determining the level of LEKTI protein expression in the sample. Absence or a substantial reduction in the level of LEKTI protein expression may be indicative of Netherton's syndrome.

In one embodiment the invention provides an in vitro method of diagnosing NS comprising contacting a sample of tissue from a patient, advantageously a skin biopsy, with a LEKTI-specific antibody and detecting or quantitatively measuring any complexes formed by binding of this antibody to LEKTI protein present in the tissue sample.

This method may be performed in any standard immunoassay format known in the art, such as an enzyme-linked immunosorbent assay (ELISA), a radioimmunoassay or the like. A typical immunoassay would involve contacting the tissue sample with the antibody to allow specific binding to any LEKTI protein present in the sample and then detecting the presence of/measuring the amount of complexes of antibody bound to LEKTI protein. As an alternative to an immunoassay, the method may be performed in an immunohistochemistry format on a sample of tissue removed from the individual. Procedures for performing immunoassays or immunohistochemistry in accordance with this aspect of the invention would be well known to the skilled artisan and are described, for example, in IMMUNOASSAY: E. Diamandis and T. Christopoulus (1996), Academic Press, Inc., San Diego, Calif.; IMMUNOCHEMISTRY 1 and 2: A practical approach (1997), A. P. Johnstone and M. W. Turner, Eds., IRL Press at Oxford University Press.

LEKTI-specific polyclonal and monoclonal antibodies, may be prepared using techniques which are known per se in the art, using a fragment of the LEKTI protein as the challenging antigen. Suitable immunogenic fragments are as follows:

CEESSTPGTTAASMPPSDE (SEQ ID NO: 13) and

EYRKLVRNGKLACTRENDP (SEQ ID NO: 14).

Peptide fragments of the LEKTI protein may be synthesised by chemical synthesis techniques (see, for example, Merrifield, R. B. (1963), Automated Synthesis of Peptides, Science 150: pp178-184). In order to elicit a strong immune response the peptide fragment (also known as a hapten) may be covalently linked to a carrier molecule such as bovine serum albumin, ovalbumin or Keyhole Limpet Hemocyanin (KLH), as is well known in the art. Anti-LEKTI polyclonal antibodies may be prepared by inoculating a host animal, such as a rabbit, with a LEKTI peptide-carrier conjugate and recovering immune serum and the same LEKTI peptide-carrier conjugate could also be used as challenging antigen for the preparation of anti-LEKTI monoclonal antibodies, according to standard techniques (see, for example ANTIBODIES: A Laboratory Manual, E. Harlow and D. Lane, Cold Spring Harbor Laboratory, Cold Spring Harbor, N.Y.; IMMUNOCHEMISTRY 1 and 2: A practical approach (1997), A. P. Johnstone and M. W. Turner, Eds., IRL Press at Oxford University Press).

Not all disease-causing mutations will lead to abolition of LEKTI protein expression. Some may lead to expression of an aberrant protein, for example a truncated form of the protein or a protein which is substantially full-length but non-functional. The invention therefore provides screens which look for the presence of an aberrant protein, for example by size determination or by using an antibody with specificity for either a mutated form or the native form of the protein, and biochemical diagnostic screens based on evaluating the serine protease inhibitor activity in a tissue sample. A protocol for performing a biochemical screen for serine protease inhibitor activity is included in the accompanying Examples. These methods are preferably carried out in vitro on tissue samples removed from the body, e.g. skin biopsies.

The above-listed genetic and biochemical screening methods of the invention may be used to identify asymptomatic heterozygote carriers of NS and to diagnose NS in patients. The availability of a diagnostic test which can be used to confirm diagnosis of NS in newborns represents significant progress as it has previously been difficult to diagnose the condition with certainty. The ability to identify asymptomatic carriers is also extremely important in providing a basis for genetic counselling in families at risk. In a further important aspect, the invention also provides for pre-natal screening for NS. Pre-natal diagnosis in early pregnancy will preferably be carried out using chorionic villus sampling, isolating fetal genomic DNA and testing the DNA using the genetic screen described above. In later stages of pregnancy, amniotic fluid would be used as a source of fetal cells and the same genetic screens would be performed on genomic DNA isolated from these cells.

The invention further provides a number of variant LEKTI nucleic acid and protein molecules and also mutant and variant SPINK5 alleles.

The variant LEKTI nucleic acid sequences provided by the invention are defined as nucleic acid molecules comprising the nucleotide sequence illustrated in SEQ ID NO: 2 (identical to the complete coding sequence of the wild-type LEKTI cDNA, including the stop codon ‘tga’) but having at least one of the following single nucleotide substitutions:

A substituted for G at position 56;

A substituted for G at position 81;

G substituted for A at position 116;

A substituted for G at position 316;

C substituted for T at position 1004;

G substituted for A at position 1103;

G substituted for A at position 1113;

A substituted for G at position 1156;

C substituted for T at position 1188;

T substituted for C at position 1257;

G substituted for A at position 1258;

T substituted for A at position 1275;

G substituted for A at position 1389;

A substituted for G at position 1556;

A substituted for C at position 1557;

T substituted for C at position 1659;

T substituted for C at position 1850;

A substituted for G at position 1859;

A substituted for G at position 2313;

A substituted for G at position 2343;

T substituted for C at position 2358;

T substituted for C at position 2368;

T substituted for C at position 2412;

G substituted for A at position 2465;

A substituted for G at position 2469;

G substituted for A at position 2472;

T substituted for G at position 2475;

C substituted for T at position 2788;

G substituted for A at position 2915; or

T substituted for C at position 3009.

The complements of these sequences are also provided.

The nucleotide positions given correspond to nucleotide positions in the LEKTI cDNA sequence, taking A of the initiating ATG codon as position 1 (this same numbering system is used throughout).

The nucleic acid molecules of the invention may also include all or a part of the 5′ and/or 3′ untranslated regions of the LEKTI cDNA. The 5′ untranslated region extends a further 43 nucleotides upstream of the initiating ATG codon. The 3′ untranslated region extends a further 289 nucleotides downstream of the stop codon.

The nucleic acid molecules of the invention may be single or double stranded RNA, single or double stranded DNA (encompassing cDNA and also recombinant DNA molecules), synthetic forms and mixed polymers, both sense and antisense strands. Furthermore, the nucleic acid molecules may be chemically or biochemically modified as will be readily appreciated by those skilled in the art. Possible modifications include, for example, the addition of isotopic or non-isotopic labels. Also included are synthetic molecules that mimic polynucleotides in their ability to bind to a designated sequence, for example via hydrogen bonding. Such molecules are known in the art and include, for example, so-called peptide nucleic acids (PNAs) in which peptide linkages substitute for phosphate linkages in the backbone of the molecule.

The invention further provides for fragments of the above-defined nucleic acid molecules, specifically sense or antisense oligonucleotides comprising at least 15 consecutive nucleotides of the above-defined nucleic acid molecules, with the proviso that this must included the specified variant base, i.e. the oligonucleotides must contain at least one single nucleotide substitution as compared to the wild-type LEKTI cDNA sequence.

Oligonucleotides corresponding to polymorphic variants occurring within the introns of the SPINK5 gene are also provided. These oligonucleotides comprise 15 or more consecutive nucleotides of the SPINK5 genomic sequence, in each case including the polymorphic base.

The oligonucleotide molecules of the invention are preferably from 15 to 50 nucleotides in length, even more preferably from 15-30 nucleotides in length, and may be DNA, RNA or a synthetic nucleic acid, and may be chemically or biochemically modified or may contain non-natural or derivatized nucleotide bases, as will be readily appreciated by those skilled in the art. Possible modifications include, for example, the addition of isotopic or non-isotopic labels, substitution of one or more of the naturally occurring nucleotide bases with an analog, internucleotide modifications such as uncharged linkages (e.g. methyl phosphonates, phosphoamidates, carbamates, etc.) or charged linkages (e.g. phosphorothioates, phosphorodithioates, etc.). Also included are synthetic molecules that mimic polynucleotides in their ability to bind to a designated sequence to form a stable hybrid. Such molecules are known in the art and include, for example, so-called peptide nucleic acids (PNAs) in which peptide linkages substitute for phosphate linkages in the backbone of the molecule. An oligonucleotide molecule according to the invention may be produced according to techniques well known in the art, such as by chemical synthesis or recombinant means.

The oligonucleotide molecules of the invention may be double stranded or single stranded but are preferably single stranded, in which case they may correspond to the sense strand or the antisense strand of the SPINK5 gene. The oligonucleotides may advantageously be used as probes or as primers to initiate DNA synthesis/DNA amplification. They may also be used in diagnostic kits or the like for detecting the presence of SPINK5 nucleic acid sequences. For example, certain of the oligonucleotides may be used in the genetic screens described herein for determining susceptibility to atopic disease. These tests generally comprise contacting the probe with a sample of test nucleic acid under hybridising conditions and detecting for the presence of any duplex or triplex formation between the probe and complementary nucleic acid in the sample. The probes may be anchored to a solid support to facilitate their use in the detection of nucleic acid sequences according to the invention. Preferably, they are present on an array so that multiple probes can simultaneously hybridize to a single sample of target nucleic acid. The probes can be spotted onto the array or synthesised in situ on the array. (See Lockhart et al., Nature Biotechnology, vol. 14, December 1996 “Expression monitoring by hybridisation to high density oligonucleotide arrays”). A single array can contain more than 100, 500 or even 1,000 different probes in discrete locations.

Where the oligonucleotides are to be used as probes and primers for genotyping, skilled artisans will appreciate that the precise length of the oligonucleotide and positioning of the polymorphic nucleotide may vary depending upon the nature of the technique to be used to perform genotyping at the polymorphic locus. For example, PCR-SSP generally requires allele-specific primers in which the polymorphic nucleotide is positioned at the extreme 3′ end, whereas techniques based on hybridisation might require allele-specific oligonucleotide probes having the polymorphic nucleotide positioned towards the middle of the probe.

The variant LEKTI protein molecules provided by the invention are defined as isolated variant LEKTI polypeptides comprising the sequence of amino acids illustrated in SEQ ID NO: 1 (the complete wild-type LEKTI amino acid sequence) but having one of the following amino acid substitutions:

Asn to Ser at position 39;

Asp to Asn at position 106;

Val to Ala at position 335;

Asn to Ser at position 368;

Asp to Asn at position 386;

Glu to Lys at position 420;

Arg to Ser at position 425;

Gly to Glu at position 519;

Arg to Lys at position 620;

Met to Ile at position 781;

Lys to Arg at position 822;

Glu to Asp at position 825;

Cys to Arg at position 930; or

His to Arg at position 972.

The mutant and variant SPINK5 alleles provided by the invention are defined as alleles having particular mutations/polymorphic variants compared to the wild-type SPINK5 allele. SPINK5 genomic sequences are are deposited in publicly accessible sequence databases. A list of accession numbers is provided in the accompanying Examples.

Thus, the invention provides an isolated mutant SPINK5 allele containing a mutation selected from the group consisting of:

81 G→A, 2258insG, 153delT, 238insG, 283−2A→T, 2468insA, 720insT, 1086delAT, 1888−1G→A, 2313G→A, 2369 C→T(R790X), 1038insG(A)₄, 1111C→T(R371X), 81+2T→A, 2459delA, 2259insA, 2240+1G→A, 81+5G→A, 1608−1G→A, 2041delAG, 649C→T(R217X), 628C→T(R210X), 56G→A or 377delAT.

The invention also provides an isolated variant SPINK5 allele containing at least one polymorphic variant selected from the group consisting of:

82-31 A→G, 316 G→A, 475-86 G→C, 1011−12 C→T, 1093−26 C→T, 1093−10 A→G, 1103 A→G, 1156 G→A, 1188 T→C, 1258 A→G, 1221−50 G→A, 1389 A→G, 1557 C→A, 1607+47 C→T, 1659 C→T, 1821−47 T→G, 1888−54 G→A, 2241−27 T→C, 2313+31 C→G, 2313+48 G→A, 2358 C→T, 2412 C→T, 2475 G→T, 2740−59 G→A, 2915 A→G, 2965−46 T→C, 1257 C→T, 1113 A→G, 3009 C→T, 283−12T→A, 475−39A→G, 1302+19G→A, 1607+49delC, 1888−14T→C, 2313+21C→G, 2539−7T→G, 2667−22insT, 2965−8C→T, 3217+23T→C and 3217+23T→G.

The nucleotide positions of the mutations/variants again correspond to the LEKTI cDNA as described by Mägert et al. 1999.

Mutations/variants occurring in an intron are numbered as a certain number of nucleotides + or − the nucleotide at the nearest exon/intron boundary.

Also provided by the invention are probe molecules which are capable of specifically hybridizing to a mutant or variant SPINK5 allele as defined above but not to the wild-type SPINK5 allele.

The invention further provides a method for identifying the disease-causing mutation (or mutations for a compound heterozygote) in a patient suffering from Netherton's syndrome or a patient suspected of suffering from Netherton's syndrome which comprises comparing the sequence of all or a part of the SPINK5 alleles carried by the patient with the wild-type SPINK5 sequence. Differences between the patient alleles and the wild-type sequence, other than conservative or silent polymorphisms, may identify a disease-causing mutation, particularly if the difference in primary nucleotide sequence leads to a frameshift or a splicing defect and/or incorporation of a premature translation termination codon.

Advantageously, regions of genomic DNA may be amplified by PCR using SPINK5-specific primer-pairs in order to provide a template for sequencing. In this case, high quality polymerase should be used to avoid introducing PCR errors during the amplification and sequence differences should be confirmed by sequencing the products of several independent PCR reactions. A list of 32 primer-pairs which may be used to amplify individual exons of the SPINK5 gene is included in the accompanying Examples.

As an alternative to DNA sequencing, other methods known in the art for identifying mutations include SSCP, heteroduplex analysis, denaturing gradient gel electrophoresis and chemical cleavage of mismatch. Commonly, differences identified on the basis of SSCP or heteroduplex analysis etc will later be confirmed by sequencing.

In a further aspect, the invention also provides therapeutic agents based on the LEKTI protein itself and functional fragments thereof and on nucleic acid sequences which encode the LEKTI protein.

The LEKTI protein itself, or fragments thereof which retain equivalent biological function, may be a useful therapeutic agent, restoring LEKTI function to cells which lack such function. Accordingly, the invention provides a substance comprising a serine protease inhibitor having the amino acid sequence illustrated in SEQ ID NO: 1 or a functional fragment thereof for use in a method of treatment of the human body by therapy.

The therapeutic agent may comprise the full length LEKTI protein, having the amino acid sequence illustrated in SEQ ID NO: 1, but may also comprise a fragment of LEKTI which retain substantially equivalent biological function to the full length LEKTI protein. The ‘biological function’ of the LEKTI protein is defined herein as its activity as a serine protease inhibitor. This activity may be conveniently measured using the in vitro trypsin inhibitory assay described herein. Therapeutically useful fragments will be those which retain biological function although this may be slightly enhanced or reduced compared to the full length protein.

Preferred fragments of the LEKTI protein for therapeutic use are fragments comprising one or several Kazal domains. Positions of the Kazal domains within the sequence illustrated in SEQ ID NO: 1 are described by Mägert et al. 1999. Particularly preferred are the Kazal domain fragments designated HF6478 (comprising amino acid residue numbers 23 to 77 of the sequence shown in SEQ ID NO: 1) and HF7665 (comprising amino acid residue numbers 356 to 423 of the sequence shown in SEQ ID NO: 1). Peptides corresponding to these fragments, assumed to be derived from a full length LEKTI precursor protein, have been isolated from human blood filtrate.

Although preferred therapeutic agents will be based on the wild-type LEKTI protein, and fragments thereof, it is also within the scope of the invention to use variants which exhibit minor variations in primary amino acid sequence but which retain substantially equivalent biological function, including LEKTI proteins encoded by naturally occurring SPINK5 allelic variants. Changes which are conservative of LEKTI function may include insertion or deletion of one or more amino acids or conservative substitution of one or more amino acids with another amino acid or acids having similar chemical characteristics, substitution with an unusual amino acid residue and also in vivo or in vitro chemical and biochemical modifications, such as acetylation, carboxylation, phosphorylation and glycosylation. The choice of amino acids for making conservative changes will be well-known to those skilled in the art.

It will be appreciated that LEKTI proteins or fragments thereof for therapeutic use can be synthesised by a number of different methods, such as chemical methods known in the art or recombinant methods.

LEKTI proteins for use in human therapy are preferably produced by recombinant DNA methods using cloned DNA fragments encoding the LEKTI protein (e.g. a cloned cDNA). As would be well known to those of ordinary skill in the art, the basic steps in the production of a recombinant protein are: provision of a DNA molecule encoding the LEKTI protein, integrating the DNA into a replicable expression vector in a manner suitable for expressing the LEKTI protein either alone or as a fusion protein, introducing the expression vector into a suitable host cell, culturing the host cell under conditions which promote expression of the LEKTI protein and recovering and substantially purifying the LEKTI protein. Details OF the construction of various cloning and expression vectors containing LEKTI sequences are included in the accompanying Examples.

Techniques for solid phase chemical synthesis of proteins are well known in the art and could be used as an alternative to the recombinant expression approach (see Merrifield, R. B. (1963), Automated Synthesis of Peptides, Science 150: pp178-184). By way of example, polypeptides may be synthesised on an Applied Biosystems 430A peptide synthesiser using reagents and protocols supplied by the manufacturer (Applied Biosystems).

Also within the scope of the invention are therapeutic agents based on functional equivalents or structural analogs of the LEKTI protein.

“Functional equivalents” of the LEKTI protein may include fusion proteins, for example fusions comprising the full length LEKTI protein or a functional fragment thereof fused either N-terminally or C-terminally to an heterologous protein or peptide fragment or fusions comprising LEKTI or a functional fragment thereof having heterologous proteins or peptide fragments fused to both the − and C-termini.

Fusion proteins will typically be made by recombinant nucleic acid techniques in which two or more open reading frames are translationally fused or may be chemically synthesized. In recombinant systems, expression of a fusion protein can provide a convenient means for purification, the heterologous protein or peptide tag commonly being removed after purification by chemical or enzymatic cleavage. A LEKTI protein or a functional fragment thereof could also be fused to a heterologous protein or peptide which imparts an additional function.

“Functional equivalents” of the LEKTI protein may also include structural analogs thereof with equivalent biological function. Biologically active LEKTI analogs can be designed and produced according to techniques known to those of skill in the art (see. e.g., U.S. Pat. Nos. 4,612,132; 5,643,873 and 5,654,276). These structural analogs can be based, for example, on a specific LEKTI amino acid sequence and maintain the relative position in space of the corresponding amino acid side chains. In a preferred embodiment, the structural analog retains the biological function of the wild-type LEKTI, as defined herein, but possesses a “biological advantage” over the corresponding wild-type LEKTI amino acid sequence with respect to one or more of the following properties: solubility, stability and susceptibility to hydrolysis and proteolysis.

Methods for preparing LEKTI structural analogs include modifying the N-terminal amino group, the C-terminal carboxyl group, and/or changing one or more of the amino linkages to a non-amino linkage. Two or more such modifications can be present in a single structural analog molecule. Modifications of peptides to produce structurally analogous molecules of equivalent biological function are described in U.S. Pat. Nos. 5,643,873 and 5,654,276.

References herein to “LEKTI protein(s)” in connection with the preparation of therapeutic agents or pharmaceutical formulations should, unless otherwise stated, be taken to include all possible functional fragments, functional equivalents and structural analogs, as defined herein.

Therapeutic agents based on LEKTI proteins and fragments thereof would be especially useful for the treatment of Netherton's syndrome and also for atopic diseases associated with reduced LEKTI function. A pharmaceutical composition in accordance with this aspect of the invention may include a therapeutically effective amount of the LEKTI protein in combination with any standard physiologically and/or pharmaceutically acceptable carriers known in the art. “Pharmaceutically acceptable” means a non-toxic material which does not interfere with the activity of the pharmaceutically active ingredients in the composition. “Physiologically acceptable” refers to a non-toxic material that is compatible with a biological system such as a cell, tissue or organism. Physiologically and pharmaceutically acceptable carriers may include diluents, fillers, salts, buffers, stabilizers, solubilizers etc. The pharmaceutical preparations of the invention are to be administered in pharmaceutically acceptable amounts, an effective amount being an amount of a pharmaceutical preparation that alone, or together with further doses, produces the desired response in the condition being treated. The precise amount of the composition administered will, however, generally be determined by a medical practitioner, based on the circumstances pertaining to the disorder to be treated, such as the severity of the symptoms, the composition to be administered, the age, weight, and response of the individual patient and the chosen route of administration.

The invention further provides for delivery of a therapeutically effective amount of a nucleic acid encoding the LEKTI protein or a functional fragment thereof, as defined above, to cells either in vivo or ex vivo in order to restore LEKTI function to cells which lack such function, i.e. somatic gene therapy. The effects of defective LEKTI expression are likely to be manifested in epithelia which is a relatively straightforward target for gene delivery.

A wide variety of different viral and non-viral delivery systems are known in the art which might be used for this purpose. One of the simplest types of delivery systems known in the art (which is useful for targeting gene delivery to epithelial cells) is a system based on the combination of a plasmid expression vector and liposomes. The plasmid expression vector should comprise nucleic acid sequences encoding the LEKTI protein or fragment thereof operably linked to a promoter which is capable of directing an appropriate level and pattern of expression, possible a tissue- or cell type-specific promoter. The term “operably linked” refers to a juxtaposition wherein the components described are in a relationship permitting them to function in their intended manner.

The invention also makes it possible to isolate the natural ligands (e.g. serine proteases) of the LEKTI serine protease inhibitor as potential targets for therapeutic intervention in the treatment of Netherton's syndrome and atopic disease, based on binding of the ligand to the LEKTI serine protease inhibitor.

There are a number of techniques known in the art which might be used to identify LEKTI ligands. For example, so-called pull-down experiments could be carried out using a preparation of the LEKTI protein (or a LEKTI protein fragment including the presumed ligand binding region) immobilised on a solid support or matrix such as, for example, chromatographic resin, polystyrene microbeads or a filter. The immobilised protein may be used to isolate molecules which bind thereto in accordance with standard procedures for affinity chromatography.

Other techniques which may be used to isolate proteins which interact with LEKTI via a direct protein-protein interaction include two-hybrid experiments, such as the classical yeast two-hybrid system described by Chien et al. 1991. This technique is routinely used in the art to identify novel protein-protein interactions and may readily be adapted to the identification of LEKTI binding partners. Vectors for use in two-hybrid experiments are commercially available from Clontech.

As aforementioned, the naturally occurring ligands of LEKTI are likely to represent attractive targets for therapeutic intervention in Netherton's syndrome and in atopy. In particular, LEKTI agonists which mimic the serine protease inhibitory effects of LEKTI may have therapeutic potential. The invention therefore contemplates methods of screening for compounds which may have potential pharmacological activity in the treatment of Netherton's syndrome and/or atopic disease, which methods comprise determining the serine protease activity of a serine protease previously identified as a ligand of the LEKTI serine protease inhibitor in the presence or absence of a candidate compound, wherein compounds which are inhibitors of the ligand are scored as having potential pharmacological activity in the treatment of atopic disease or Netherton's syndrome.

The serine protease inhibitory activity of potential LEKTI agonists may also be tested in the trypsin inhibition assay described in the accompanying Examples.

In a final aspect, the invention provides the isolated promoter region of the SPINK5 gene encoding the LEKTI serine protease inhibitor, comprising the complete nucleotide sequence illustrated in SEQ ID NO: 3 (or the sequence shown in SEQ ID NO: 3 excluding any sequences downstream of the LEKTI transcription initiation site). Also contemplated by the invention are fragments of the complete sequence shown in SEQ ID NO: 3 which retain promoter activity, in particular the ability to direct a tissue-specific pattern of gene expression, most preferably a tissue-specific gene expression pattern substantially identical to the native LEKTI gene expression pattern, and also fragments which function as enhancer elements. The promoter and/or enhancer activity of fragments of the sequence shown in SEQ ID NO: 3 can be easily tested using reporter gene assays, for example by constructing a deletion series of the complete fragment. Plasmid vectors containing reporter genes for use in testing the promoter and/or enhancer activity of DNA fragments are available commercially (e.g. the pGL2 and pGL3 vector series from Promega Madison, Wis., USA). Promoter elements required for positioning of RNA polymerase and initiation of basal transcription are likely to be found immediately upstream of the transcription initiation site, whereas enhancer elements and elements required for tissue-specific expression might be found far upstream.

In addition to the functional promoter activity studies outlined above, knowledge of the sequence of the SPINK5 promoter sequence is useful for the construction of homologous recombination vectors for in vivo targeting of SPINK5 in the mouse by promoter sequence alterations, characterisation of keratinocyte or T-cell specific DNA responsive elements (RE) for targeted expression of transgenes, characterisation of responsive elements related to inflammation and/or immunologic mediators, characterisation of transcription factors and/or signalling pathways related to inflammation and/or immunologic mediators and design of possible inhibitors/blocking agents of the RE, pathways or factors mentioned above.

Isolation of the promoter region of the SPINK5 gene allows the development of reporter gene assays to identify compounds which modulate SPINK5 gene expression. Accordingly, the invention provides a method of identifying a compound with potential pharmacological activity in the treatment of atopic disease or Netherton's syndrome, which method comprises:

providing a recombinant host cell containing a reporter gene expression construct comprising the promoter region of the human SPINK5 gene operably linked to a reporter gene;

contacting the host cell with a candidate compound; and

screening for expression of the reporter gene product.

The above method of the invention can be used to identify compounds which up-regulate the expression of SPINK5 and hence have potential pharmacological activity in the treatment of Netherton's disease or atopic disease.

For the purposes of this application, the term “promoter region of the human SPINK5 gene” may refer to the complete sequence shown in SEQ ID NO: 3, a fragment thereof lacking sequences downstream of the transcription initiation site or a transcriptionally active fragment thereof, to the proximal promoter region, i.e. sequences immediately upstream of the LEKTI transcription start site which are necessary for correctly positioning RNA polymerase and also to the proximal promoter region plus any additional sequence elements which may be involved in regulating LEKTI gene expression, e.g. upstream enhancer sequences etc.

the promoter region of the human SPINK5 gene, as defined above, is positioned to control expression of a reporter gene encoding a protein product which is directly or indirectly detectable. The juxtaposition of the SPINK5 promoter region and a reporter gene may be referred to herein as a ‘reporter gene expression construct’.

Reporter genes which may be used in accordance with the invention include those which encode a fluorescent product, such as green fluorescent protein (GFP) or other autonomous fluorescent proteins of this type or those which encode an enzyme product, such as for example chloramphenicol acetyl transferase (CAT), β-galactosidase and alkaline phosphatase, which is capable of acting on a substrate to produce a detectable product.

Reporter gene assays using reporter gene expression constructs are well known in the art and commonly used in the art to test the promoter activity of a given DNA fragment. They may also be adapted, as in the present invention, to screen for compounds capable of modulating gene expression.

The reporter gene expression construct is preferably incorporated into a replicable expression vector so that it may be conveniently introduced into the eukaryotic host cell. The eukaryotic host cell must be one which contains the appropriate transcription machinery for RNA Polymerase II transcription, and is preferably a cultured mammalian cell. In a preferred embodiment, the host cell is a cell type which is known to express LEKTI in vivo or is a transformed cell line derived from a cell type known to express LEKTI in vivo.

An expression vector may be inserted into the host cell in a manner which allows for transient transfection or alternatively may be stably integrated into the genome of the cell (i.e. chromosomal integration). Chromosomal integration is generally preferred for drug screening because the expression constructs will be maintained in the cell and not lost during cell division, also there is no need to separately control for the effects of copy number.

Stable integration of a reporter gene expression construct into the genome of eukaryotic host cell may be achieved using a variety of known techniques. The most simple approach is selection for stable integration following transfection of a host cell with a plasmid vector. Briefly, a plasmid vector comprising a reporter gene expression construct consisting of the LEKTI promoter region ligated to a promoterless reporter gene cDNA and also a gene encoding a dominant selectable marker, such as neomycin phosphotransferase, is first constructed using standard molecular biology techniques. The plasmid vector is then used to transfect eukaryotic host cells using one of the standard techniques such as, for example, lipofection. Following transfection stable cell lines in which the plasmid DNA has become randomly integrated into the chromosome are selected with growth on appropriate media. For plasmids carrying the neomycin phosphotransferase gene this is achieved using the antibiotic G418. Plasmid vectors suitable for use in the construction of stable cell lines are commercially available (for example the pCI-neo vector from Promega corporation, Madison Wis., USA).

Stable integration into mammalian chromosomes may also be achieved by homologous recombination, a technique which has been commonly used to achieve stable integration of foreign DNA into embryonic stem cells as a first stage in the construction of transgenic mammals. Stable integration into eukaryotic chromosomes can also be achieved by infection of a host cell with a retroviral vector containing the appropriate reporter gene expression construct.

It will be appreciated that a wide variety of compounds can be tested using the method of the invention to see whether they are capable of up-regulating SPINK5 gene expression and hence have potential pharmacological activity. The compound may be of any chemical formula and may be one of known biological or pharmacological activity, a known compound without such activity or a novel molecule such as might be present in a combinatorial library of compounds. The method of the invention may be easily adapted for screening in a medium-to-high throughput format.

Compounds which are identified as being capable of up-regulating SPINK5 gene expression should be further tested in order to establish whether the effect on gene expression is SPINK5-specific or non-specific. This could be achieved using a control cell containing a control reporter gene expression construct with no SPINK5 promoter sequences.

The present invention will be further understood with reference to the following non-limiting experimental examples and the accompanying drawings in which:

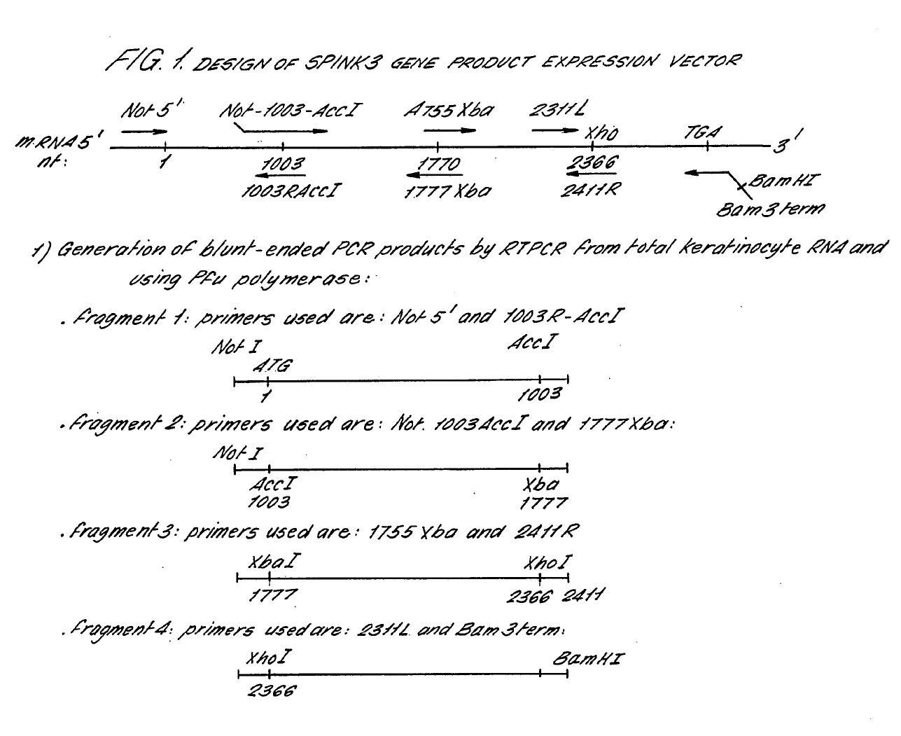

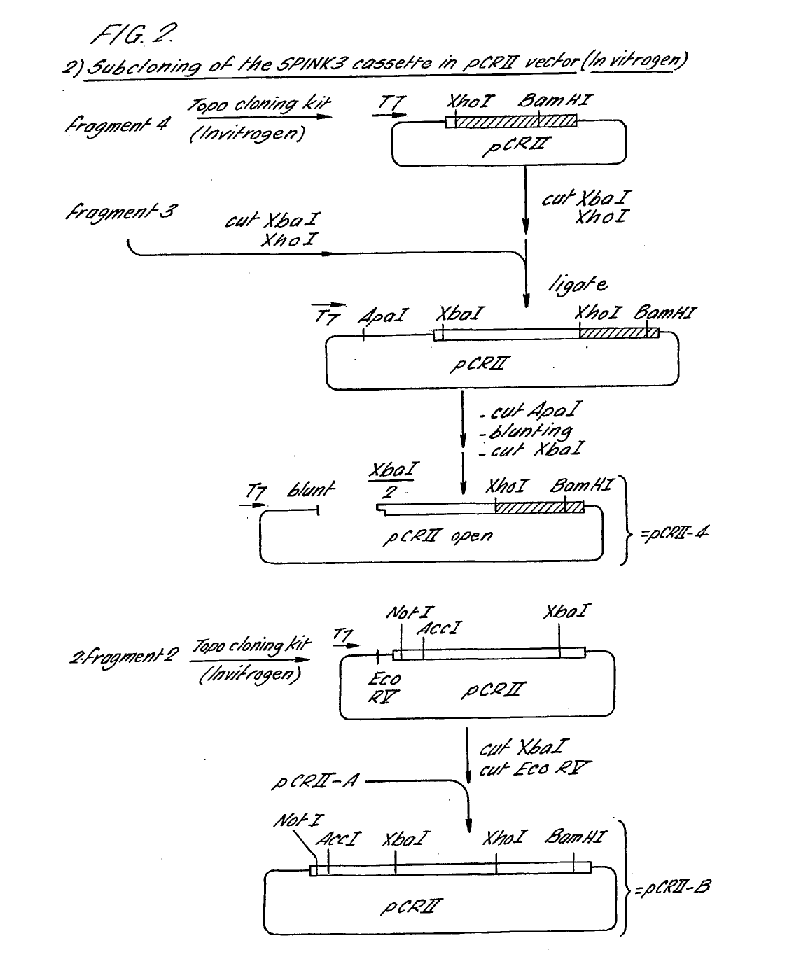

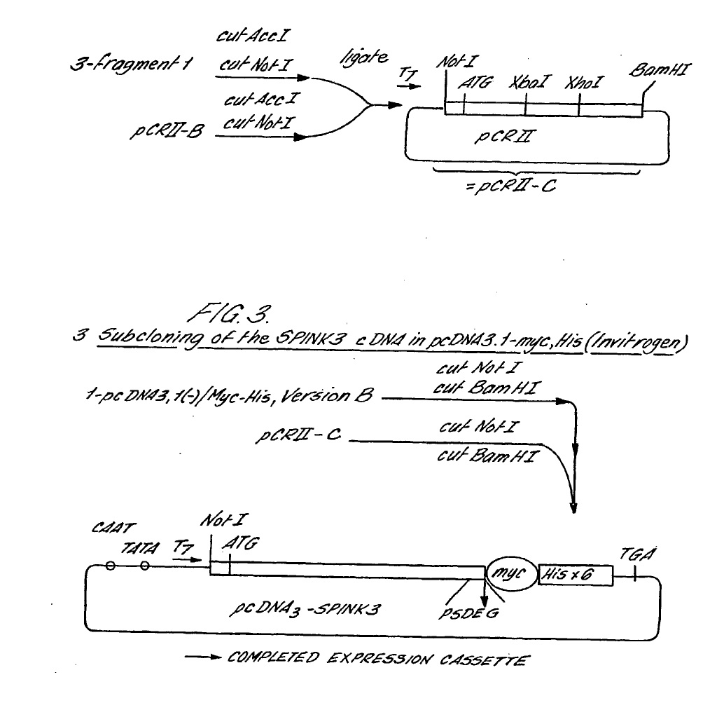

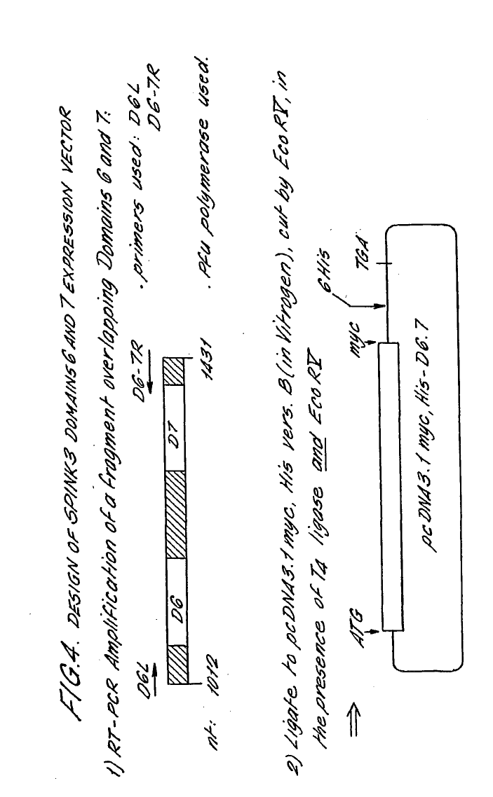

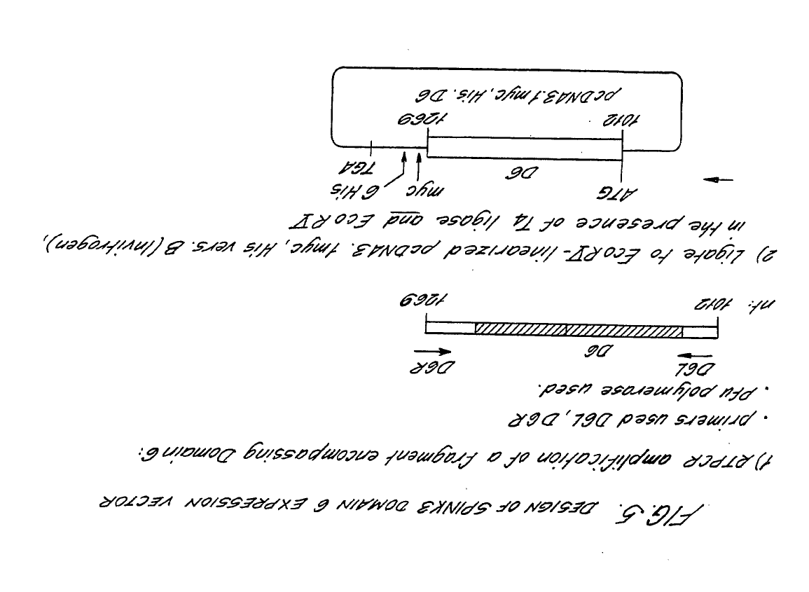

FIGS. 1 to 5 schematic diagrams of the cloning strategy for cloning of DNA sequences encoding the LEKTI protein and various fragments thereof into expression vectors.

### EXAMPLE 1

Search for Candidate Genes or Transcripts in the NS Gene Locus.

Database searching of the consensus map Genemap '99 identified 8 genes and 38 ESTs in the D5S463-D5S2013 interval. Of these genes, PDGFRB, SPARC and CDX1 which encode the Platelet-Derived Growth Factor Receptor β, osteonectin and the caudal type homeo box transcription factor 1, respectively, had been mapped distal to BT1 on the published physical maps of the Diastrophic Dysplasia and the Treacher Collins syndrome regions (Hastbacka et al. 1994; Superti-Furga et al. 1996), and thus could be excluded from the NS region. Of the remaining 5 genes, none appeared to be obvious candidates for the NS gene. Two were involved in human diseases sharing no common features with NS: the PDE6A gene which encodes the α subunit of retinal rod cGMP phosphodiesterase is mutated in autosomal recessive retinitis pigmentosa (Huang et al. 1995); and the DTDST gene coding for a transmembrane sulfate transporter is defective in Diastrophic Dysplasia and achondrogenesis type 1B (Hastbacka et al. 1994; Superti-Furga et al. 1996). The three other genes encode the casein kinase Iα (CSNK1A1), the adrenergic receptor β2 (ADRB2) and the regulator of mitotic spindle assembly (RMSA1). The casein kinase Iα is an ubiquitously expressed serine/threonine protein kinase which is involved in the regulation of G-protein-coupled receptors (Tobin et al. 1997), cell cycle (Gross et al. 1997), and DNA and RNA synthesis (Ceglieska and Virshup 1993). Polymorphisms in ADRB2 have been associated with susceptibility to nocturnal asthma and obesity (Turki et al. 1995; Large et al. 1997), and targeted disruption of the ADRB2 gene in the mouse leads to abnormal vascular tone and impaired energy metabolism during exercise (Chruscinski et al. 1999). Lastly, the RMSA1 gene product is required for spindle assembly and chromosomal segregation (Yeo et al. 1994).

Computer-assisted EST walking was performed for all of the 38 non-redundant ESTs mapping within the NS interval, using the Medical Research Council Human Genome Mapping Project (HGMP) EST-blast program. ESTs were found with homologies to the serine-protease inhibitor precursor VAKTI, the P1 chain of alcohol dehydrogenase class II, the chemokine RANTES, the leulkosialin precursor, and the rat arylsulfatase B (stSG28807, WI-22716, stSG53883, H14645, stSG15286, respectively). These ESTs were detectable by RT-PCR analysis from cultured keratinocytes and were therefore further evaluated.

EST stSG53883 which maps within the NS linkage interval was found to be a fragment of a previously reported cDNA encoding a 1064-residue serine protease inhibitor designated LEKTI (Mägert et al. 1999). LEKTI exhibits a peculiar organisation in 15 highly homologous modules (D1-D15), two of which (D2, D15) perfectly match the Kazal serine protease inhibitor pattern C—(X)ₙ,—C—(X)₇—C—(X)₁₀—C—(X)_{2/3}—C—(X)ₘ—C, where the cysteine residues C are involved in three disulfide bonds in a 1-5,2-4, 3-6 pattern (Laskowski and Kato, 1980). Peptides derived from domains D1 and D6 have been detected in blood, and domain D6 has been reported to have antitrypsin activity (Mägert et al. 1999). Serine protease inhibitors are down-regulators of the cellular and extracellular proteases which play a pivotal role in cell communication, extracellular matrix remodelling (Werb, 1997) and apoptosis (Solary et al. 1998). Some serine protease inhibitors also exert a paracrine/autocrine role independently of their antiprotease activity; this has been reported for 3,4-dichloroisocoumarin which down-regulates the NF-KB pathway in Jurkat cells (Rossi et al. 1998).

The expression pattern of LEKTI transcripts was investigated by Northern blot analysis of human tissues and cultured epidermal keratinocytes with a specific LEKTI cDNA probe. An approximately 3.7 kb hybridisation signal was obtained with the thymus and keratinocyte extracts only. Since nonsense-mediated decay (NMD) of mutated transcripts is frequently observed in recessive diseases, the levels of LEKTT mRNA in cultured keratinocytes from NS patients was also investigated. A dramatic decrease in transcript levels was observed on Northern blots of extracts from of 6 NS patients tested, suggesting impaired stability of LEKTI transcripts in these patients. A search was therefore initiated for molecular defects within the cDNA encoding LEKTI in NS patients.

The intron/exon layout of the new gene encoding LEKTI was elucidated and the new gene was named SPINK5 for serine protease inhibitor, Kazal-type, 3, according to the Human Gene Nomenclature Committee (http://www.gene.ucl.ac.uk/hugo/). SPINK5 spans approximately 60 KB, comprises 33 exons and 32 introns. In contrast the SPINK1 and SPINK2 genes encoding the two other human Kazal serine protease inhibitors, namely the pancreatic secretory trypsin inhibitor (PSTI) (Horii et al. 1987), and the acrosin-trypsin inhibitor (HUSI-II) (Moritz et al. 1993) respectively, both comprise 4 exons, suggesting SPINK5 has a distinct phylogenetic origin. The sequences of all exons and intronic boundaries have been submitted to the EMBL database (http://www.ebi.ac.uk/embl/index.html)

### EXAMPLE 2

Analysis of Mutations in Netherton's Syndrome Families

Mutation analysis identified a total of 12 different mutations in 13 families (Table 1a). At least, 10 out of the 12 mutations generate premature termination codons of translation (PTC), while consequences of 2 of the 4 splice site mutations have not been fully assessed. These mutations include a nonsense mutation (R790X), four mononucleotide insertions (238insG, 720insT, 2258insG, 2468insA), three mono/dinucleotide deletions (153delT, 2458delG, 1086 delAT), and four splice site mutations (81G→A, 238−2A→T, 1888−1G→A, 2313G→A). Mutations 2468insA and 153delT occurred within five and ten mononucleotide repeats, respectively, and may result from slipped mispairing at the replication fork. The consequences of the splice site mutations at the mRNA level were investigated using mRNA prepared from patient keratinocytes grown in vitro. Mutation 1888−1G→A disrupts the intron 20 acceptor splice site and activates a cryptic splice site located 1 nucleotide downstream, causing deletion of guanosine 1888. Mutation 2313G→A occurs at the last base of exon 24 and results in skipping of this exon (73 bp). Mutations 81G→A and 283−2A→T alter the last base of exon 2 and the acceptor splice site of exon 5, respectively, and therefore are expected to alter splicing (Aebi et al. 1986). These results predict NMD of the mutated transcripts, resulting in null alleles of SPINK5 in NS patients, consistent with the recessive inheritance of this disease.

Mutation 153delT is a thymidine insertion within exon 3 that creates an Xmn I site. Mutation R790X in is a cytidine to thymidine transition which generates a premature termination codon of translation (PTC) TGA. Mutation 2313G→A occurs at the wobble position of codon 771 and alters exon 24 last base, resulting in skipping of exon 24 (73-nt).

Screening for SPINK5 mutations in 21 additional, unrelated, families with Netherton's syndrome resulted in the identification of 18 distinct mutations, 13 of which were novel. The mutations present in these families are listed in Table 1b.

Four different nonsense mutations were identified in these families (R210X, R217X, R317X and R790X). They all result from a C→T deamination at a CGA arginine codon, leading to a TGA premature termination codon.

Four small deletions and four insertions were characterised in the coding region. All of these created a shift in the reading frame. The majority occurred within mononucleotide repeats of 3 to 10 A, T or G. (153delT, 238insG, 2259insA, 2459delA, 2459insA). Other mutations included two nucleotides deletions (377delAT, 2041delAG) and the duplication of a G(A)₄ sequence.

Six different splice site mutations were identified, four of which altered the invariant splice-donor or acceptor site consensus sequences GT (81+2T→A; 2240+1G→A) or AG(1608−1G→A; 1888−1G→A). Another mutation altered the last nucleotide of exon 1 (56G→A) whilst a further was located within intron 2 at position +5 (81+5G→A). The effect of mutations 2240+1G→A, 1888−1G→A and 81+5G→A on splicing was studied using RT-PCR products from total RNA extracted from cultured keratinocytes.

Mutation 2240+1G→A was shown to result in removal of the last 59 nucleotides of exon 23 caused by activation of the cryptic splice-donor site GT within exon 23. The resulting frameshift creates a TGA stop codon, 8 amino acids downstream of the cryptic splice-site.

Mutation 1888−1G→A leads to deletion of guanosine 1888 through the activation of a cryptic splice site AG, located one nucleotide downstream of the mutation. This leads to a PTC (premature termination codon) 85 amino acids downstream of the cryptic splice site.

Mutation 81+5G→A leads to retention of the first 103 nucleotides of intron 103 through activation of a cryptic splice-donor site GT within intron 2. This creates a frameshift introducing a TGA stop codon 8 amino acids downstream of exon 2.

Mutations 81+2T→A, 1608−1G→A and 56G→A all affect consensus or highly conserved splice site sequences and are therefore likely to impair splicing.

SPINK5 is the first serine protease inhibitor primarily involved in a skin disorder. Given the hypokeratotic features of NA skin (Fartasch et al. 1999; Hausser et al. 1996), SPINK5 could be a key regulator of the keratinocyte terminal differentiation. It is assumed that loss of expression of SPINK5 results in protease-antiprotease imbalance and impaired modulation of proteolysis in the integument.

Experimental Methods for Examples 1 and 2

Biological Samples and Cells

Human keratinocytes from a control and NS patients were isolated from skin biopsies and cultured on lethally irradiated feeder-layers of 3T3-J2 murine fibroblasts in a mixture (3:1) of DMEM and Ham's F12 medium (Life-Technologies) containing 10% fetal calf serum, 5 μg/ml insulin, 0.4 μg/ml hydrocortisone, 0.1 nM cholera toxin, 10 ng/ml epidermal growth factor, 2 nM triiodothyronin and 0-18 mM adenine (Rheinwald and Green, 1975). Total RNA was purified from actively growing keratinocytes according to published procedures (Chomczynski and Sacchi, 1987). Genomic DNA was extracted from peripheral blood following standard techniques after informed consent was obtained.

Northern Blot Analysis

A 1.0 kb probe specific for EST stSG28807 was generated by digestion of 5 μg of the corresponding IMAGE clone 825-d18 with Not I and Eco RI according to the manufacturer's recommendations (New England Biolabs). A 983 bp probe specific to GAPDH cDNA was generated by RT-PCR amplification of total RNA purified from control human keratinocytes, using the primer pair 5′-AGATCCCCTCCAAAATCAAGT-3′ and 5′-TAGGCCCCTCCCCTCTTCA-3′. Probes were α³²P-random-labelled using the Prime-It™ RT kit according to the manufacturer's procedure (Stratagene), and hybridized to human keratinocyte total RNA (30 μg) northern blots and to a 12 lane human multiple tissue northern blot (Clontech).

SPINK5 Exon-Intron Organisation

A BLAST search for sequence similarities between the LEKTI cDNA sequence and the unfinished High Throughput Genomic Sequences (htgs) database revealed that the 123,434 bp human chromosome 5 clone CIT978SKB_{—}94F21 encompassed the gene encoding LEKTI.

The intron/exon organisation of SPINK5 was determined by a combination of electronic search and sequencing of long PCR products from human BAC 94F21 (containing SPINK5) and genomic DNA. SPINK5 is encoded by 33 exons and spans a region of 61 kb. Table 6 lists the size and starting positions of each exon, together with flanking intronic sequences and intron sizes. Fragments of the SPINK5 genomic sequence, including each of the exons, are included as SEQ ID Nos: 3 to 12.

Mutation Analysis of SPINK5

SPINK5 individual exons and corresponding intronic boundaries were PCR-amplified from genomic DNA (100 ng) using 32 oligonucleotide primer pairs designed on the basis of SPINK5 organization. When RNA from NS patients was available, first-strand SPINK5 cDNA was synthesized from 10 μg of total RNA in the presence of 500 ng of oligodeoxythymidine primers and 0.5 μl of SuperScript II reverse transcriptase as recommended by the manufacturer (Life-Technologies). The full ORF of SPINK5 was subsequently amplified using 0.5 μl of reverse transcription product as a template and 9 overlapping sets of specific cDNA oligonucleotide primers. No skin biopsy could be obtained from the patients in families 1 and 5. All PCR reactions were performed in a final volume of 50 μl in the presence of 3 μl of AmpliTaq DNA polymerase (Perkin-Elmer). The PCR cycling conditions were: 94° C., 3 min; 94° C., 10 sec; 58° C., 40 sec; 72° C., 20 see (30 cycles). After amplification, approximately 50 ng of the PCR products were processed for heteroduplex analysis by conformation-sensitive gel electrophoresis as described elsewhere (Ganguly et al. 1993). Amplimers showing electrophoretic mobility shifts were directly sequenced in forward and reverse orientations using an ABI PRISM Big Dye Terminator Cycle Sequencing Kit (Perkin Elmer). To sequence insertions or deletions, PCR products were subcloned into a pTOPO.2.1 vector according to the manufacturer's recommendations (InVitrogen), and multiple clones were sequenced. During the search for mutations, four conservative polymorphisms were detected in patients and control DNA: 1113A→G (arg 371), 1257C→T (gly 439), 2358T→C (leu 786) and 3009C→T (gly 1003).

In the 21 additional patients screening for mutations was accomplished by amplifying the entire coding sequence, flanking intron boundaries and 490 bp of proximal promoter region with 35 sets of primers using genomic DNA as template and then analysing the amplified products for the presence of mutations using HPLC.

GenBank Accession Numbers

SPINK5 submitted; LEKTI cDNA: AJ228139; GAPDH cDNA: M33197; Homo sapiens chromosome 5 clone CIT978SKB_{—}94F21: AC008722; SPINK1: AH001527(cDNA), M22971, M20528, M20529, M20530; SPINK2: M91438.

TABLE 1a
Characteristics of SPINK5 mutations identified
in 13 Netherton's Syndrome families.

| | | | | | Verification |
|---|---|---|---|---|---|
| Family | Mutation | Nucleotide change | Consequence | Location | method |
| 1 | 81G→A | AGgttagt→AAgttatg | Altered splicing | Exon 2 | CSGE + |
| | | | | | sequencing |
| | 2258insG | AGAA→AGGAA | Frameshift | Exon 24 | +Mn/I |
| | | | (PTC + 3) | | |
| 2 | 153delT | A(T)₆C→A(T)₅C | Frameshift | Exon 3 | +XmnI |
| | | | (PCT + 4) | | |
| 3 | 153delT | A(T)₆C→A(T)₅C | Frameshift | Exon 3 | +XmnI |
| | | | (PCT + 4) | | |
| 4 | 238insG | AGGGC→AGGGGC | Frameshift | Exon 4 | +NlaIV |
| | | | (PCT + 18) | | |
| 5 | 283-2A→T | cagCTG→ctgCTG | Altered splicing | Exon 5 | −PvuII |
| | 2468insA | T(A)₁₀G→T(A)₁₁G | Frameshift | Exon 26 | CSGE + |
| | | | (PTC + 4) | | sequencing |
| 6 | 720insT | CACG→CATCG | Frameshift | Exon 9 | CSGE + |
| | | | (PTC + 4) | | sequencing |
| 7 | 1086delAT | TCATATG→TCATG | Frameshift | Exon 12 | +NlaIII, |
| | | | (PTC + 6) | | −NdeI |
| 8 | 1888-1G→A | tagGAG→taaGAG | Altered splicing | Intron 20 | +MseI |
| | | | (PTC + 85) | | |
| 9 | 2313G→A | AGgtgagt→AAgtgagt | Skip exon 24 | Exon 24 | −HphI |
| | | | (PTC + 10) | | |
| 10 | R790X | ACTCGA→ACTTGA | Nonsense | Exon 25 | −XhoI |
| 11 | 2468insA | T(A)₁₀G→T(A)₁₁G | Frameshift | Exon 26 | CSGE + |
| | | | (PTC + 4) | | sequencing |
| 12 | 2468insA | T(A)₁₀G→T(A)₁₁G | Frameshift | Exon 26 | CSGE + |
| | | | (PTC + 4) | | sequencing |
| 13 | 2468insA | T(A)₁₀G→T(A)₁₁G | Frameshift | Exon 26 | CSGE + |
| | | | (PTC + 4) | | sequencing |

TABLE 1b
Characteristics of SPINK5 mutations in 21
further Netherton's Syndrome patients.

| | | | | | Verification |
|---|---|---|---|---|---|
| Patient | Mutation | Nucleotide change | Consequence | Location | method |
| 1 | 1038insG(A)₄ | G(A)₄AGG→ | Frarneshift | Exon 12 | Sequencing |
| | | G(A)₄G(A)₄AGG | (PTC + 31aa) | | |
| | R371X | 1111C→T | Nonsense (PTC) | Exon 13 | −TaqI |
| 2 | R371X | 1111C→T | Nonsense (PTC) | Exon 13 | −TaqI |
| | 81 + 2T→A | CAGgtt→CAGgat | Altered splicing | Intron 2 | Sequencing |
| 3 | ? | | | | |
| | 2459delA | (A)₁₀G→(A)₉G | Frameshift | Exon 26 | Sequencing |
| | | | (PTC + 26aa) | | |
| 4 | 2459delA | (A)₁₀G→(A)₉G | Frameshift | Exon 26 | Sequencing |
| | | | (PTC4 + 26aa) | | |
| | ? | | | | |
| 5 | 2259insA | (A)₆T→(A)₇T | Frameshift | Exon 24 | Sequencing |
| | | | (PTC + 3aa) | | |
| | R790X | 2368C→T | Nonsense | Exon 25 | −TaqI |
| 6 | ? | | | | |
| | 2240 + 1G→A | ATTgt→ATTat | Altered splicing | Intron 23 | Sequencing |
| | | | (PTC + 8aa) | | |
| 7 | 2240 + 1G→A | ATTgt→ATTat | Altered splicing | Intron 23 | Sequencing |
| | | | (PTC + 8aa) | | |
| | 81 + 5G→A | CAGgttag→CAGgttaa | Altered splicing | Intron 2 | +MseI |
| | | | (PTC + 8aa) | | |
| 8 | 1608−1G→A | agCAA→aaCAA | Altered splicing | Intron 17 | Sequencing |
| | 2459delA | (A)₁₀G→(A)₉G | Frameshift | Exon 26 | Sequencing |
| | | | (PTC + 26aa) | | |
| 9 | 238insG | AGGGC→AGGGGC | Frameshift | Exon4 | +NlaIV |
| | | | (PCT + 18) | | |
| | ? | | | | |
| 10 | 1888−1G→A | tagGAG→taaGAG | Altered splicing | Intron 20 | +MseI |
| | | | (PTC + 85) | | |
| | 1888−1G→A | tagGAG→taaGAG | Altered splicing | Intron 20 | +MseI |
| | | | (PTC + 85) | | |
| 11 | 2041delAG | GAGGA→GGA | Frameshift | Exon 22 | Sequencing |
| | | | (PTC + 26aa) | | |
| | R371X | 1111C→T | Nonsense (PTC) | Exon 13 | −TaqI |
| 12 | 238insG | AGGGC→AGGGGC | Frameshift | Exon4 | +NlaIV |
| | | | (PCT + 18) | | |
| | R217X | 649C→T | Nonsense (PTC) | Exon 8 | −TaqI |
| 13 | R210X | 628C→T | Nonsense (PTC) | Exon 8 | Sequencing |
| | R210X | 628C→T | Nonsense (PTC) | Exon 8 | Sequencing |
| 14 | 2459insA | (A)₁₀G→(A)₁₁G | Frameshift | Exon 26 | sequencing |
| | | | (PTC + 6aa) | | |
| | 2459insA | (A)₁₀G→(A)₁₁G | Frameshift | Exon 26 | sequencing |
| | | | (PTC + 6aa) | | |
| 15 | 81 + 2T→A | CAGgtt→CAGgat | Altered splicing | Intron 2 | Sequencing |
| | 153delT | A(T)₆C→A(T)₅C | Frameshift | Exon 3 | +XmnI |
| | | | (PCT + 4) | | |
| 16 | 153delT | A(T)₆C→A(T)₅C | Frameshift | Exon 3 | +XmnI |
| | | | (PCT + 4) | | |
| | 153delT | A(T)₆C→A(T)₅C | Frameshift | Exon 3 | +XmnI |
| | | | (PCT + 4) | | |
| 17 | 153delT | A(T)₆C→A(T)₅C | Frameshift | Exon 3 | +XmnI |
| | | | (PCT + 4) | | |
| | 153delT | A(T)₆C→A(T)₅C | Frameshift | Exon 3 | +XmnI |
| | | | (PCT + 4) | | |
| 18 | 153delT | A(T)₆C→A(T)₅C | Frameshift | Exon 3 | +XmnI |
| | | | (PCT + 4) | | |
| | 153delT | A(T)₆C→A(T)₅C | Frameshift | Exon 3 | +XmnI |
| | | | (PCT + 4) | | |
| 19 | 153deIT | A(T)₆C→A(T)₅C | Frameshift | Exon 3 | +XmnI |
| | | | (PCT + 4) | | |
| | 153delT | A(T)₆C→A(T)₅C | Frameshift | Exon 3 | +XmnI |
| | | | (PCT + 4) | | |
| 20 | 56G→A | AGG→AGA | Altered splicing | Exon 1 | +HphI |
| | ? | | | | |
| 21 | R790X | 2368C→T | Nonsense | Exon 25 | −TaqI |
| | 377delAT | ATATG-ATG | Frameshift | Exon 5 | −NdeI |
| | | | (PTC + 7aa) | | |

### EXAMPLE 3

Trypsin Inhibition Assay

Inhibitory effects of trypsin of purified, native or recombinant SPINK5 gene product, and selected domains of the SPINK5 gene product are examined in 50 mM Tris-Hcl buffer, pH 8.0, containing 150 mM NaCl, and 0.01% (v/v) Triton X-1000. Na-benzoyl-L-arginine p-nitroanilide (final concentration 220 mM) is used as a substrate, its hydrolysis may be monitored by the change in absorbance at 405 nm. Various inhibitor and trypsin concentrations are added to the reaction mixtures (according to standard assay optimisation procedures) and the residual activity of the proteinase is measured in a quartz cuvette thermostatically controlled at 25° C. Bovine albumin (fraction V) is used as a negative control. Aprotinin is used as a control.

This assay may be used to assess the inhibitory activity of SPINK5 gene products or selected domains of the SPINK5 gene product or of potential agonists of the SPINK5 gene product.

### EXAMPLE 4

Design and Synthesis of Recombinant SPINK5 Gene Product Expression Vectors.

The construction of expression vectors containing various fragments of the LEKTI cDNA is illustrated schematically in FIGS. 1 to 5.

An expression vector for expression of the SPINK5 gene product was constructed as follows:

1) Generation of Blunt-Ended PCR Fragments of the LEKTI cDNA by RT-PCR from Total Keratinocyte RNA Using Pfu Polymerase:

Fragment 1: primers Not-5′ and 1003R-AccI

Fragment 2: primers Not-1003L-AccI and 1777R-Xba

Fragment 3: primers used are 1755L-Xba and 2411R

Fragment 4: primers used are 2311L and Bam-3term

2) Subcloning of the SPINK5 Cassette in pCRII (Invitrogen).

A complete LEKTI cDNA was assembled in the vector pCRII. PCR fragment 4 was first cloned between the XhoI and BamHI sites of pCRII using the Topo cloning kit (Invitrogen) and the resulting clone cut with XbaI and XhoI. PCR fragment 3 was then cut with XbaI and XhoI and ligated into the XbaI/XhoI cut pCRII vector. The resulting construct (fragments 3 and 4 in pCRII) was cut with ApaI, the cut ends blunted and then re-cut with XbaI to generate intermediate vector pCRII-A having one blunt end and one XbaI end at the 5′ end of fragment 3. PCR fragment 2 was then cloned into pCRII using the Topo cloning kit (Invitrogen). The resulting construct (PCR fragment 2 in pCRII) was cut with XbaI and EcoRV and the insert ligated into pCRII-A to generate pCRII-B (PCR fragments 2, 3 and 4 in pCRII). pCRII-B and PCR fragment 1 were then both cut with AccI and NotI and then ligated to form pCRII-C (PCR fragments 1, 2, 3 and 4 in pCRII vector).

3) Subcloning of the SPINK5 cDNA in pcDNA3.1-myc,His (Invitrogen).

Vector pcDNA3.1(−)/Myc-His, Version B (Invitrogen) was cut with NotI and BamHI. Vector pCRII-C generated in part 2) was also cut with NotI and BamHI to release the SPINK5 cDNA fragment which was then ligated into the cut pcDNA3.1 vector to give pcDNA3-SPINK5.

An Expression Vector Containing SPINK5 Domains 6 and 7 was Constructed as Follows:

A PCR product spanning domains 6 and 7 of the LEKTI cDNA (nt: 1012-1431) was generated by RT-PCR using the primers D6L and D6-7R and Pfu polymerase. The resulting product was ligated into pcDNA3.1 Myc,His Version B (Invitrogen), cut by EcoRV, in the presence of T4 ligase and EcoRV. The ligated product was denoted pcDNA3.1 Myc,His-D6,7.

An Expression Vector Containing SPINK5 Domain 6 was Constructed as Follows:

A PCR product spanning domain 6 of the LEKTI cDNA (nt: 1012-1269) was generated by RT-PCR using the primers D6L and D6R and Pfu polymerase. The resulting product was ligated into pcDNA3.1 Myc,His Version B (Invitrogen), cut by EcoRV, in the presence of T4 ligase and EcoRV. The ligated product was denoted pcDNA3.1 Myc,His-D6.

The pcDNA3.1 Myc,His-based constructs described above all feature a myc-epitope tag and a 6-Histidine tag fused with the carboxy-terminal end for immunopurification or immunodetection of the expressed polypeptide.

The cloning vectors used in the above cloning strategy are all commercially available (e.g. from Invitrogen).

Sequences of the oligonucleotides designed for use in the construction of these vectors are as follows:

| | |
|---|---|
| Not-5′: | GCGGCCGCTTCAACATGAAGATAG |
| 1003R-AccI: | ATTTTCTGCTTGGAAGTAGACTTG |
| Not-1003L-Acc: | GCGGCCGCAACGTATACTTCCAAGCAGAAAAT |
| 1755L-Xba: | GATCCTATTGAGGGTCTAGATG |
| 1777R-Xba: | CATCTAGACCCTCAATAGGATC |
| Bam-3term: | CCGTCTGACGAAGGGGATCCG |
| 2411R: | ACCATGTGTCTTGCCATCTGG |
| 2311L: | AAGGATACATGTGATGAGTTTAG |
| D6L: | GTACTCTTAGATTGTCTTTTGTT |
| D6-7R: | AAGAAGGCCTCACACATGGA |
| D6R: | GTACTCTTAGATTGTCTTTTGTT |

These expression vectors are useful for:

production of recombinant SPINK5 gene product

production of recombinant SPINK5 gene product selected domains

structural studies leading to peptide-mimicking/agonist design or target prediction

production of antibodies raised against the recombinant proteins/domains

functional studies

### EXAMPLE 5

Linkage and Association of the Netherton's Locus in Families with Atopic Dermatitis.

The SPINK5 gene was sequenced in 18 unrelated individuals with atopic dermatitis. The sequencing identified a number of coding and non-coding single nucleotide polymorphisms (Table 2). Three of the five coding polymorphisms were found in exon 13 and exon 14, corresponding to domain 6 of the LEKT1 protein.

Linkage and association were sought to the phenotypes of atopy (defined as the presence of an elevation of the total serum IgE and/or a positive titre of the serum IgE against house dust mite or grass pollen and/or a positive prick skin test >3 mm> control for the same antigens), asthma, atopic dermatitis (eczema) and the total serum IgE concentration.

Evidence for linkage of the total serum IgE to microsatellite markers and SNPs was found (Table 3). The linkage was confined to maternal alleles. Maternal effects are a distinctive feature of allergic disease, and linkage and association through maternally derived alleles has been observed in these and other families at other loci influencing atopy.

Linkage was not seen to the categorical phenotypes of atopy, asthma and atopic dermatitis, reflecting the lower power to detect linkage to categorical traits which are common in the population.

Four of the coding polymorphisms (Asn368Ser, Asp3B6Asn, Glu420Lys and His972Arg) were typed in the 150 families and tested for association with the presence of atopic disease and phenotypes underlying atopy (Table 4a). The transmission disequilibrium test of Weinberg was used to allow for parent of origin effects. Asn368Ser and Glu420Lys were significantly associated with atopy (Table 4a). The polymorphisms also showed association with asthma and with a positive titre of the serum IgE against house dust mite (RAST HDM, Table 4a). Only weak or absent associations were seen with atopic dermatitis.

Overall, the results show that allele 1 of Asn368Ser and allele 1 of Glu420Lys were both associated with an increased risk of atopy and atopic asthma when maternally inherited. The relative risk was approximately four compared to the same allele when paternally inherited. Asn368Ser and Glu420Lys were in almost complete linkage disequilbrium, and forming their combined haplotypes did not add any power to tests of association. It was also not possible to differentiate which of the two polymorphisms might be responsible for the associations seen.

Asp386Asn and His972Arg were not significantly associated with any phenotype. However, they were of low frequency, and significance testing consequently would have lacked power.

Examination of the separate transmission of maternal and paternal alleles shows that individual alleles have apparently opposing effects when inherited paternally or maternally (Table 4b). Very similar parent of origin effects have been observed at the FcεRI-β locus in these same families. The findings are suggestive of genomic imprinting. However, Netherton's disease is a Mendelian recessive disorder in which imprinting is not observed. Differential expression of maternal and paternal alleles remains a possibility if it is tissue-specific, for example in the thymus, and if it takes place at particular moments during immune development.

Although the results show that variation in SPINK5 modifies the risk of atopy and asthma in children with atopic dermatitis, no mechanism for disease in these subjects is immediately apparent. A defect in mucosal surfaces is possible, particularly as many allergens are proteases. In support of this is the association between Asn368Ser and Glu420Lys and IgE titres against House Dust Mite (Table 4a). Mast cells also produce proteases, with incompletely defined functions. Alternatively, the expression of the SPINK5 gene within the thymus may indicate a role in T-cell maturation, or with antigen handling within other thymic cells. Early life events seem critical in the establishment of allergic disease, and a differential action of maternal and paternal alleles may be more likely within the developing thymus than at mucosal or epithelial surfaces.

Experimental Methods for Example 5

Subjects and Methods

Two panels of families were examined. The first panel (Panel A) contained 60 nuclear families comprising 277 individuals, who were recruited from the dermatology clinics at the Great Ormond Street Hospital for Children through a single proband with active atopic dermatitis. Panel B contained 88 families comprising 402 individuals who had been recruited from out patient clinics at GOSH on the basis of at least two first degree relatives with active atopic dermatitis. A questionnaire, which included the diagnostic criteria for atopic dermatitis defined by the UK working Party and a set of questions based on the American Thoracic Society's questionnaire for asthma and allergic rhinitis was completed for each individual. Each family was examined for evidence of atopic dermatitis by a doctor, as previously described. Skin test responses to house dust mite (Dermatophagoides pteronyssinus), timothy grass (Phleum pratense), -alternaria (Alternaria alternata), cat dander (Felis domesticus), egg white and cow's milk was carried out on all individuals in Panel A. The total of specific IgE to the same panel of allergens was measured by a fluorescent enzyme immuno-assay (Pharmacia CAP system, Pharmacia, Uppsala, Sweden) in both panels of families. Atopy was defined as the presence for positive skin prick test response 3 mm>negative control, or positive specific IgE, or raised total serum IgE or any combination of these features (as previously described).

Fluorescent Genotyping Using Microsatellite Markers

One of each oligonucleotide PCR primer pair was labelled with one of three fluorescent dye molecules: HEX, FAM or NED. PCR amplification conditions were determined for each marker using the following PCR reaction mixture: 200 mM dNTPs, 1× PCR buffer (AmpliTaq buffer, Cetus Corp., USA), 25 μg of each PCR primer, 0.5-3.0 mM Magnesium Chloride, 0.04 units of AmpliTaq (Cetus Corp., USA) and 50 ng of genomic DNA. Samples were placed in Costar 96-well plates, overlayed with mineral oil and PCR was carried out using an MJ Research PTC-200 machine. PCR amplification conditions were; 5 minutes at 94° C., then 35 cycles of 45 seconds at 94° C., 45 seconds at 40-55° C., 30 seconds at 72° C. and a final extension of 5 minutes at 72° C. Three of the primer pairs were obtained from public databases: D5S2090, D5S434, and D5S413. Two microsatellites (SC_CA and SC_IMP) were identified from the sequence within the ND gene and the following PCR primer pairs were used to amplify them:

| | |
|---|---|
| CA-F | GAACAATTTGATAATGGTGTG |
| CA-R | AAGAATCCTAAGCACAATGTG |
| IMP-F | ACTATTCCATTGGAAAGGAG |
| IMP-R | GGGTGTGTGAGTTGAGATGG |

PCR products were pooled with GS-500 ROX-labelled molecular weight markers (Applied Biosystems (ABI), UK) and size separated on 6% (w/v) denaturing polyacrylamide gels using an ABI 373 sequencing machine. Microsatellite alleles were then sized using the ABI GeneScan Analysis and Genotyper software programs.

Identification of SNPs Within the ND Gene

Individual exons were amplified using PCR with the PCR primer pairs shown in Table 5. The PCR mix was as follows: 1× Amplitaq Gold™ KCl buffer, 800 μM each dNTP, 2.5%M Magnesium Chloride, 0.2 μM forward primer, 0.2 μM reverse primer, 1.3 units AmpliTaq Gold made up to 50 μl with sterile water. Samples were placed in Costar 96-well plates, overlayed with mineral oil and PCR was carried out using an MJ Research PTC-200 machine. PCR cycle conditions were as follows: 95° C. for 15 minutes then 30 cycles of 95° C. for 30 seconds, 55-58° C. for 30 seconds and 72° C. for 30 seconds with a final 72° C. for 7 minutes. Products were analysed on 2%(w/v) agarose gels and visualised using a Stratagene Eagle Eye II system.

The PCR product was purified away from excess primers and dNTPs using the QIAgen QIAquick columns according to the manufacturer's recommended protocol. Purified PCR products were sequenced using a cycle sequencing protocol based upon the Big Dye terminator chemistry (ABI, UK). The cycle sequencing mix consisted of 1 μl of 10 mM sequencing primer, 2 μl of Big Dye mix, 2 μl of half-Big Dye mix and 5 μl of purified PCR product. This mix was placed in a capped thin-walled 0.2 ml microtube and PCR was carried out using an MJR PTC-200 machine. The PCR cycle conditions were as follows: 95° C. for one minute, then 35 cycles of 95° C. for 10 seconds, 50° C. for 10 seconds and 60° C. for 4 minutes.

Cycle sequencing products were precipitated using ethanol to remove excess unincorporated fluorescent label. Precipitated products were resuspended in 3 μl of gel loading buffer (0.8%(w/v) Blue Dextran, 8 mM EDTA, 85% (v/v) deionised formamide), heated at 95° C. for 3 minutes and then placed immediately on ice. 2.5 μl of each sample was then loaded on a 4%(w/v) denaturing polyacrylamide gel and electrophoresed using an ABI 377 sequencer. Cycle sequencing products were sized using the ABI Sequencing Analysis program and electropherograms exported for analysis using PHRED and PHRAP to determine sequence quality and the presence of SNPs.

SNP Typing by Enzyme Digestion of PCR Products

The Glu420Lys polymorphism was typed by Hph I digestion of exon 14 PCR product, using the primers in Table 5. The final concentrations in the PCR mix were: 1× AmpliTaq Gold KCl buffer, 250 μM dNTP mix, 2.5 mM Magnesium Chloride, 0.3 μM 14L primer, 0.3 μM 14R primer, 1-2 units of Amplitaq Gold enzyme, made up to 15 μl with sterile water. Samples were placed in Costar 96-well plates, overlayed with mineral oil and PCR was carried out using an MJ Research PTC-200 machine. The PCR cycle conditions were 95° C. for 18 minutes, 38 cycles of 95° C. for 1 second, 55C for 20 seconds, 72° C. for 5 seconds, followed by a final 72° C. for 10 minutes. 5 μl of PCR reaction together with 1× NEBuffer 4 and 1.25 units of Hph I were made up to a final volume of 10 μl with sterile water and incubated at 37° C. for 2 hours and run out on a 2% (w/v) agarose gel.

The His972Arg polymorphism was typed by Fok I digestion of exon 26 PCR product, using the primers in Table 5. The final concentrations in the PCR mix were: 1×AmpliTaq Gold KCl buffer, 250 μM dNTP mix, 2.5 mM Magnesium Chloride, 0.1 μM 26L primer, 0.1 μM 26R primer, 1.2 units of Amplitaq Gold enzyme, made up to 10 μl with sterile water. Samples were placed in Costar 96-well plates, overlayed with mineral oil and PCR was carried out using an MJ Research PTC-200 machine. The PCR cycle conditions were 94° C. for 5 minutes, 10 cycles of 94° C. for 40 seconds, 58° C. (reduced by 0.5° C. per cycle) for 40 seconds, 72° C. for 30 seconds, followed by 30 cycles of 94° C. for 40 seconds, 55° C. for 40 seconds, 72° C. for 30 seconds, and a final 72° C. for 5 minutes. 5 μl of PCR reaction together with 1× NEBuffer 4 and 1 unit of Fok T were made up to a final volume of 10 μl with sterile water and incubated at 37° C. for 2 hours and run out on a 2% (w/v) agarose gel.

SNP Typing by Oligonucleotide Ligation Assays

The polymorphisms Asn368Ser and Asp386Asn were typed by the oligonucleotide ligation assay (OLA) (Tobe et al. 1996) using PCR products of a region spanning exons 13 and 14 of the gene. PCR reactions were performed with 50 ng of DNA, 200 μM of each dNTP, 0.8 μM of each primer (13L and 14R, Table 5), 2.5 mM magnesium chloride, 1× AmpliTaq Gold KCl buffer and 0.75 units of AmpliTaq Gold DNA polymerase. The final PCR volume was 15 μl. PCR cycling conditions were 95° C. for 15 minutes followed by 35 cycles of 95° C. for 1 minute, 58° C. for 1 minute and 72° C. for 1 minute. A final extension of 72° C. for 10 minutes was included.

After successful amplification, genotyping of the PCR products was performed by OLA using a Beckman Biomek 2000. The protocol was almost identical to the published one with a couple of modifications (Tobe et al. 1996). 15 μl PCR products as opposed to 20 μl were diluted with 50 μl of distilled water containing 0.1% Triton X-100. Ligation temperatures for the two polymorphisms were 56° C. for Asn368Ser and 62° C. for Asp386Asn.

Sequences of the OLA primers used to type the two polymorphisms were as follows:—

Asn368Ser: Fluorescein-^{5′}TCGGCAGGAGCTTTGCAG^{3′}, Digoxigenin-^{5′}TCGGCAGGAGCTTTGCAA^{3′}Phosphorylated-^{5′}TGAATATCGAAAGCTTGTGAG^{3′}-Biotin

Asp386Asn: Fluorescein-^{5′}GCTTGCACCAGAGAGAACG^{3′}Digoxigenin-^{5′}GCTTGCACCAGAGAGAACA^{3′}Phosphorylated-^{5′}ATCCTATCCAGGGCCCAG^{3′}-Biotin

The antibodies used to detect the ligation products were alkaline-phosphatase labelled anti-digoxigenin and horseradish peroxidase labelled anti-fluorescein. Genotypes were scored colorimetrically using a Beckman PlateReader and the ARC software. Individuals of known genotype were included as controls on each 96 well plate.

TABLE 2
Polynwrphisms identified within the Netherton's gene

| Exon/Intron | SNP* | Amino Acid | Frequency** |
|---|---|---|---|
| Intron 2 | 82−31: A to G | | 0.38 A |
| Exon 3 | 116: A to G | Asn39Ser | 3.6% |
| Intron 5 | 283−12: T to A | | 3.6% |
| Exon 5 | 316: G to A | Asp106Asn | 0.03 A |
| Intron 6 | 475−39: A to G | | 7.1% |
| | 475−86: G to C | | 0.44 G |
| Exon 11 | 1004: T to C | Val335Ala | 14.3% |
| Intron 11 | 1011−12: C to T | | 0.50 T |
| Intron 12 | 1093−26: C to T | | 0.47 C |
| | 1093−10: A to G | | 0.47 A |
| Exon 13 | 1103: A to G | Asn368Ser | 0.50 A |
| | 1156: G to A | Asp386Asn | 0.13 A |
| | 1188: T to C | His396 | 0.47 T |
| Intron 13 | 1221−50: G to A | | 0.50 A |
| Exon 14 | 1258: A to G | Glu 420 Lys | 0.48 G |
| | 1275: A to T | Arg425Ser | 3.6% |
| Intron 14 | 1302+19: G to A | | 10.7% |
| Exon 15 | 1389: A to G | Gly 463 | 0.50 G |
| Exon 17 | 1556: G to A | Gly519Glu | 3.6% |
| | 1557: C to A | Gly 519 | 0.22 A |
| Intron 17 | 1607+47: C to T | | 0.09 T |
| | 1607+49: delC | | 3.6% |
| Exon 18 | 1659: C to T | Val 553 | 0.50 T |
| Intron 19 | 1821−47: T to G | | 0.09 G |
| Exon 20 | 1850: C to T | Ala 617 | 10.7% |
| | 1859: G to A | Arg620Lys | 3.6% |
| Intron 20 | 1888−14: T to C | | 42.8% |
| | 1888−54: G to A | | 0.38 G |
| Intron 23 | 2241−27: T to C | | 0.44 T |
| Intron 24 | 2313+21: C to G | | 3.6% |
| | 2313+31: C to G | | 0.34 C |
| | 2313+48: G to A | | 7.1% |
| | 2343: G to A | Met781Ilc | 3.6% |
| Exon 25 | 2358: C to T | Leu 786 | 0.44 C |
| | 2412: C to T | Gly 804 | 0.44 C |
| Exon 26 | 2465: A to G | Lys822Arg | 3.6% |
| | 2475: G to T | Glu 825 Asp | 0.08 T |
| | 2469: G to A | Lys 823 | 3.6% |
| | 2472: A to G | Lys 824 | 3.6% |
| Intron 26 | 2539−7: T to G | | 3.6% |
| Intron 27 | 2667−22: insT | | 3.6% |
| Intron 29 | 2740−59: G to A | | 0.50 A |
| Exon 29 | 2788: T to C | Cys930Arg | 3.6% |
| Exon 30 | 2915: A to G | His 972 Arg | 0.10 G |
| Intron 30 | 2965−8: C to T | | 3.6% |
| | 2965−46: T to C | | 0.44 T |
| Exon 31 | 3009: C to T | Gly 1003 | 21.4% |
| Intron 33 | 3217+23: T to C | | 21.4% |
| | 3217+23: T to G | | 3.6% |

*Alleles are numbered according to the published cDNA (GenEmbl accession number AJ228139). Where they are intronic, they are numbered + or − the nucleotide at the nearest exon-intron boundary.
**Frequency is expressed as frequency minor allele (by convention, allele 2).

TABLE 3
Linkage to Total serum IgE

| Maternally derived Alleles | | |
|---|---|---|
| Marker | θ | In(IgE) |
| D5S2090 | 0.005 | — |
| D5S434 | 0.001 | — |
| SC_CA | 0.001 | — |
| SC_IMP | 0.000 | 0.05 |
| Asn368Ser | 0.000 | 0.006 |
| Asp386Asn | 0.000 | 0.1 |
| Glu420Lys | 0.000 | 0.004 |
| His972Arg | 0.156 | 0.02 |
| D5S413 | 0.225 | 0.006 |

Table 4.

Tests of Association

TABLE 4
Weinberg Test
Parent of origin effects included in model

| Marker | Phenotype | p | R1 | R2 | Rm* |
|---|---|---|---|---|---|
| Glu420Lys | Atopy | 0.005 | 0.58 | 0.26 | 4.04 |
| Glu420Lys | Asthma | 0.012 | 0.56 | 0.27 | 3.84 |
| Glu420Lys | Eczema | 0.09 | 0.86 | 0.49 | 2.98 |
| Glu420Lys | RAST Index | 0.023 | 0.63 | 0.23 | 3.78 |
| Glu420Lys | RAST HDM | 0.014 | 0.55 | 0.18 | 4.78 |
| Asn368Ser | Atopy | 0.023 | 0.63 | 0.37 | 3.47 |
| Asn368Ser | Asthma | 0.024 | 0.54 | 0.35 | 3.67 |
| Asn368Ser | Eczema | ns | | | |

*R1 = Relative risk of 1, 2 heterozygote compared to 1, 1 homozygote
R2 = Relative risk of 2, 2 homozygote compared to 1, 1 homozygotes
Rm = Relative risk of maternal allele 1 compared to paternal allele 1

TABLE 4b
Transmission Disequilibrium Test

| +UZ,5/ 14 Atopy and Glu420Lys | | |
|---|---|---|
| | | Not |
| Allele | Transmitted | Transmitted |
| Paternal | 18 | 33 |
| | 33 | 18 |
| Maternal | 34 | 20 |
| | 20 | 34 |

TABLE 5
Oligonucleotide sequences used for PCR amplification and
sequencing of the ND gene

| | | | Size |
|---|---|---|---|
| Exon | Forward Primer (L) | Reverse Primer (R) | (bp) |
| 1 | TATGCATGGAGTGGACCTGTA | AGACATTTCAGGATTATACATGC | 224 |
| 2 | GTGCCCTTCTTTTATTTGCCATG | AAAGCTATTAGTACCTACCAG | 248 |
| 3 | TATCTACTATGTATCAGGCATTC | ATTTACCAGTTCAGAGACTAGC | 371 |
| 4 | ATCTGGGGTTCTGTGTCCAC | CAGGTATGACCTAGTAATTAAG | 327 |
| 5 | TATTAGCTCAATGTAGCCTTC | TGTAGGGAAAATTGTGTCATG | 349 |
| 7 | AGTACTTACTGAGCTACATCTAC | TTATCTCTCTGCTGAGTGATTC | 358 |
| 9 | GGAAGGATCTCTGAGCCTAG | AGTTTCCTTTCAAAGTTATTTTTAC | 376 |
| 11 | GCGATGTGCTCTAAAGATTCG | CTCACTTCCCTGTCTTGAGC | 438 |
| 12 | GAAGAAATCATAGCACCATAC | GCAAAATCTCACCCTTTAGGC | 349 |
| 13 | GAGATGTAACATTAGTTTCTGC | ATGTCTCCAATCAGACAGTTTCTC | 370 |
| 14 | TGCAATTGTGAGGATTTCACAG | CCTGAACATGATCTGTGGATC | 304 |
| 15 | AATCCATGCCTTCAAAGTTAATC | CCAAGACTGAATGCTACACTG | 392 |
| 16 | TGAGGCAGGAGAATTGCTTG | TTATCAGGACCCTTTTTGTTC | 366 |
| 17 | CTGATTGATGACGGAAGCTTTG | CTCACGGTCTATACTCTAC | 389 |
| 18 | CAAAAGCACCTCTCAGACTAG | AGTACTCTTGTATATGGGGATC | 380 |
| 19 | TTCCAAATGTGTGACCTAGTTC | CAAACTCAGTGCAAAGACAGC | 432 |
| 20 | GTTTACCTTTCCACTCCAAAGC | TGAACCCCTAGTTCCCTCAC | 319 |
| 21 | GAAATACAGCCACCTTCTTAAG | AATACATATTATTTGCCTGGCTC | 392 |
| 22 | AGAGAAGGCCTGCAGCATAG | AGCACTGCGTTTACCATAGAC | 344 |
| 23 | CCACCGTACCCGGCAATG | TGCATGGAAGGAGCCATA | 360 |
| 24 | GTGCCAGAATCCTAGGAAGAC | CTAGCTTGATCATGTTGACACC | 332 |
| 25 | ATTATTTTGCCTATCACAGCAAG | TCATTAATTACCAGATCTGCTTC | 372 |
| 26 | TGACTGTGAGTCTTAAAGTAC | GGGACAGAGTCAGCATTTCAC | 252 |
| 27 | TCTGTTTTTTTCCTGTGTTATGAGT | GTGTGATGCCAAGTATCTTAGG | 335 |
| 28 | CCAACAATCAGAACTGATTAGC | CAAAGTGACAAGACAAGAAAAATC | 389 |
| 29 | GGCCTCTGTTGCCAGGATG | TATACAAACTTATTCAAGCAGCAG | 347 |
| 30 | GCCTGGGCCTTCCAAAAT | ACTTCAGGCTGCACTGAATCA | 379 |
| 31 | GTGCAAAATCAATCTTTGAGTTTG | TTATATCAGTGCATTACTATG | 379 |
| 32 | GACACCTGGATGATACCTAC | CCAGATAAATGTCCATTACTCAG | 365 |

PCR products were not obtained from exons 6, 8, and 32

TABLE 6
SPINK5 exon-intron organisation

| | | Exon | | Intron |
|---|---|---|---|---|
| Exon | 5′ splice donor site | size (bp) | 3′ splice acceptor site | size (Kb) |
| 1 | none (5′ UTR) | 55 | ATACAAGG₅₅gtgagcaatttgtgtg | 1.241 |
| 2 | tatattttcatcccagA₅₆TGCTGCC | 26 | AAGATCAG₈₁gttagtcctgcttttt | 4.873 |
| 3 | tttggcattatcttagG₈₂AAATGTG | 129 | ATGATACT₂₀₉gtgagtaaaggtttct | 1.697 |
| 4 | tcttgtccttttccagG₂₁₀GAAAAAG | 73 | CAACAGAG₂₈₂gtgagactatttggag | >8.0 |
| 5 | atcatgtcttttgcagC₂₈₃TGAATTG | 128 | GAGAATGC₄₁₀gtgagtattctctgaa | ≅1.7 |
| 6 | taacttttgattctagG₄₁₁AAAACCG | 64 | CAGAGCAG₄₇₄gtgaggtcaattgtca | 0.840 |
| 7 | tttccctgttcttcagG₄₇₅ATGTATG | 128 | GAGCTGTT₆₀₂gtaagtagcatcatcc | 0.880 |
| 8 | ttacttttcttaacagT₆₀₃TTAAAAG | 65 | CTGAAAAG₆₆₇gtaaaatgactcacca | 2.725 |
| 9 | aattgtcttttcaaagG₆₆₈ATTTTTG | 127 | GAAATTTT₇₉₄gtgagtatagaagtgg | ≅1.3 |
| 10 | attttactttttccagC₇₉₅AAGCGGC | 88 | AAATTGTG₈₈₂gtgagaatcagtttga | ≅1.5 |
| 11 | cttattcattattcagA₈₈₃AACTCTG | 128 | GCCTACTT₁₀₁₀gtgagtatagagtttt | 1.239 |
| 12 | tttccctcttattcagC₁₀₁₁CAAGCAG | 82 | CATATGCA₁₀₉₂gtgagtggaatccatc | 1.138 |
| 13 | tcttctatctcggcagG₁₀₉₃AGCTTTG | 128 | GTCTTCTT₁₂₂₀gtgagtagccctgcag | 0.773 |
| 14 | aaccatccttttttagC₁₂₂₁CAAGCAG | 82 | CCTTTGAG₁₃₀₂gtgagtttatatatcctc | 0.344 |
| 15 | tgcctcaatttcacagG₁₃₀₃AGTTGTG | 127 | GCCTTCTT₁₄₂₉gtgagtagagcagtgag | 3.043 |
| 16 | tttcggtttcttaaagT₁₄₃₀CAACAAG | 50 | CTGCAAAG₁₄₇₉gtaatattctcaggaa | 2.036 |
| 17 | tttcttcatttcccagG₁₄₇₉AAATCTG | 128 | AGTGTGTT₁₆₀₇gtgagtgtccacccca | 1.588 |
| 18 | tctttttctattacagC₁₆₀₈AAACTTG | 85 | CAGTTCAG₁₆₉₂gtagttgtttgagatc | 2.930 |
| 19 | ctgcttcatttggcagG₁₆₉₃AGCTGTG | 128 | GCCTTCTT₁₈₂₀gtgagtgggcggcagc | 0.972 |
| 20 | ctcccttttcttatagC₁₈₂₁CAGCAAG | 67 | CTGAAAAG₁₈₈₇gtagtaatcctgaatg | 1.427 |
| 21 | tttcttttccttttagG₁₈₈₈AGACATG | 128 | GCAGTCTT₂₀₁₅gtgagtgcacaaagaa | 1.880 |
| 22 | actttctaatttccagC₂₀₁₆CAGAAAG | 97 | ACACTCAG₂₁₁₂gtgagagcaacctcta | 1.970 |
| 23 | tcttctctgttttcagG₂₁₁₃ACGAATG | 128 | GCAAAATT₂₂₄₀gtaagtatttctctcaa | 0.421 |
| 24 | cttttttctcctccagG₂₂₄₁GAAAGAG | 73 | CAGGGAAG₂₃₁₃gtgagttattttttgg | 0.950 |
| 25 | cttcccatcttttcagG₂₃₁₄ATACATG | 128 | GAAAAACT₂₄₄₁gtgagtatgtttcaaa | 0.158 |
| 26 | attgttttccccccagG₂₄₄₂GAAAGGG | 97 | ACAAAGAG₂₅₃₈gtaatagatgttagac | 3.441 |
| 27 | ctgctactgttggtagG₂₅₃₉ATCTGTG | 128 | AGCATCTT₂₆₆₆gtacgtaaaaaggttt | 0.804 |
| 28 | ttttattattctgcagT₂₆₆₇GATCGAG | 73 | ATGCAAAG₂₇₃₉gttatttattaaagga | 0.885 |
| 29 | ctttctcattttctagG₂₇₄₀ATGAGTG | 128 | GCTGTCTT₂₈₆₇gtgagtaagaggattc | 1.132 |
| 30 | ttattttcttctctagT₂₈₆₈CTAACAG | 97 | GTTCTCTG₂₉₆₄gtaaggaggactattt | 4.179 |
| 31 | ttttttgcttcttcagG₂₉₆₅ATTCTGA | 131 | GAAAACCT₃₀₉₅gtaagtattcaagttg | 2.407 |
| 32 | ctctgatctgttttagG₃₀₉₆ATACGCC | 91 | CCCCGTCT₃₁₅₈gtaagtacataagtag | 3.176 |
| 33 | ccatcttctcttctagG₃₁₅₇ACGAATG | ND | none (3′ UTR) | ND |

First and last nucleotide of each exon are numbered following the cDNA sequence, with nucleotide in position l assigned to the first nucleotide of the ATG initiation codon in exon 1. Bases in exons are denoted by underlined uppercase letters and bases in introns by lower case letters. UTR: untranslated; ND: not determined.

ADDITIONAL GENBANK ACCESSION NUMBERS

| | |
|---|---|
| LEKTI cDNA and protein | AJ228139 |
| EXON 1 | AJ391230 |
| EXON 2 | AJ270994 |
| EXON 3 | AJ391231 |
| EXON 4 | AJ391232 |
| EXON 5 | AJ391233 |
| EXON 6 | AJ391234 |
| EXON 7 | AJ391235 |
| EXON 8 | AJ276579 |
| EXON 9 | AJ391236 |
| EXON 10 | AJ276580 |
| EXON 11 | AJ391237 |
| EXON 12 | AJ391238 |
| EXON 13 | AJ391239 |
| EXON 14 | AJ391240 |
| EXON 15 | AJ391241 |
| EXON 16 | AJ276578 |
| EXON 17 | AJ391242 |
| EXON 18 | AJ391243 |
| EXON 19 | AJ391244 |
| EXON 20 | AJ391245 |
| EXON 21 | AJ391246 |
| EXON 22 | AJ391247 |
| EXON 23 | AJ391248 |
| EXON 24 | AJ391249 |
| EXON 25 | AJ391250 |
| EXON 26 | AJ391250 |
| EXON 27 | AJ391251 |
| EXON 28 | AJ391251 |
| EXON 29 | AJ391251 |
| EXON 30 | AJ391252 |
| EXON 31 | AJ391253 |
| EXON 32 | AJ391254 |
| EXON 33 | AJ276577 |

BAC clone 94F21 which contains SPINK5 gene AJ27094, AJ391230-54, AJ276577-80 and AC008722

SEQUENCE LISTING

| | |
|---|---|
| SEQ ID NO: 1 | amino acid sequence of the full length |
| | wild-type human LEKTI protein. |
| SEQ ID NO: 2 | nucleotide sequence of the coding |
| | region of the full length wild-type |
| | human LEKTI cDNA |
| SEQ ID NO: 3 | CONTIG 11 of clone CIT978SKB_94F21 |
| | spanning exons 1-4 of SPINK5 |
| SEQ ID NO: 4 | fragment of CONTIG 8 of clone |
| | CIT978SKB_94F21 spanning exon 5 of |
| | SPINK5 |
| SEQ ID NO: 5 | fragment of CONTIG 8 of clone |
| | CIT978SKB_94F21 spanning exon 6 of |
| | SPINK5 |
| SEQ ID NO: 7 | fragment of CONTIG 2 of clone |
| | CIT978SKB_94F21 spanning exon 7 of |
| | SPINK5 |
| SEQ ID NO: 8 | fragment of CONTIG 2 of clone |
| | CIT978SKB_94F21 spanning exon 8 of |
| | SPINK5 |
| SEQ ID NO: 9 | fragment of CONTIG 2 of clone |
| | CIT978SKB_94F21 spanning exon 9 of |
| | SPINK5 |
| SEQ ID NO: 10 | fragment of CONTIG 2 of clone |
| | CIT978SKB_94F21 spanning exon 10 of |
| | SPINK5 |
| SEQ ID NO: 11 | fragment of CONTIG 12 of clone |
| | CIT978SKB_94F21 spanning exons 11-32 of |
| | SPINK5 |
| SEQ ID NO: 12 | fragment of CONTIG 12 of clone |
| | CIT978SKB_94F21 spanning exon 33 of |
| | SPINK5 |
| SEQ ID NO: 13 | LEKTI peptide |
| SEQ ID NO: 14 | LERTI peptide |

### REFERENCES

1. Tobin, A. B. et al. Stimulus-dependent phosphorylation of G-protein-coupled receptors by casein kinase Ia. J. Biol. Chem. 272, 20844-20849 (1997).

2. Chavanas, S. et al. Localisation of the Netherton syndrome gene to chromosome 5q32 linked analysis and homozygosity mapping. Am. J. Hum. Genet. In press, (2000)

3. Magert, H. J. et al. LEKTI, a novel 15-domain type of human serine proteinase inhibitor. J. Biol. Chem. 274, 21499-21502 (1999).

4. Fartasch, M., Williams, M. L. & Elias, P. M. Altered lamellar body secretion and stratum corneum membrane structure in Netherton syndrome. Arch Dermatol. 135, 828-832 (1999).

5. Hausser, I. & Anton-Lamprecht, I. Severe congenital generalized exfoliative erythroderma in newborns and infants: a possible sign of Netherton syndrome. Ped Dermatol. 13, 183-199 (1996).

6. Frazer, K. A. et al. Computational and biological analysis of 680 kb of DNA sequence from the human 5q31 cytokine gene cluster region. Genome Res. 7, 495-512 (1997).

7. Meyers, D. A. et al. Evidence for a locus regulating total serum IgE levels mapping to chromosome 5. Genomics 23, 464-470 (1994).

8. Cookson, W. The alliance of genes and environment in asthma and allergy. Nature 402, B5-B10 (1999).

9. Rioux, J. D. et al. Familial eosinophilia maps to the cytokine gene cluster on human chromosomal region 5q31-q33. Am. J. Hum. Gen. 63, 1086-1094 (1998).

10. Laskowski, M. J. & Kato, I. Protein inhibitors of proteinases. Annu. Rev. Biochem. 49, 593-626 (1980).

11. Werb, Z. ECM and cell surface proteolysis: regulating cellular ecology. Cell 91, 439-442 (1997).

12. Solary, E., Eymin, B., Droin, N. & Haugg, M. Proteases, proteolysis, and apoptosis. Cell Biol. Toxicol. 14, 121-132 (1998).

13. Rossi, A., Elia, G. & Santoro, M., G. Activation of the heat shock factor 1 by serine protease inhibitors, an effect associated with nuclear factor-kappa B inhibition. J. Biol. Chem. 273, 16446-16452 (1998).

14. Gross, S. D. et al. A casein kinase I isoform is required for proper cell cycle progression in the fertilized mouse oocyte. J. Cell Sci. 110, 3083-3090 (1997).

15. Ceglieska A. and Virshup, D. M. Control of simian virus 40 replication by the HeLa cell nuclear kinase casein kinase I. Mol. Cell. Biol. 13, 1202-1211 (1993)

16. Horii, A. et al. Primary structure of human pancreatic secretory trypsin inhibitor (PSTI) gene. Biochem. Biophys. Res. Commun. 149, 635-641 (1987).

17. Moritz A., Grzeschik, K. H., Wingender, E. & Fink, E. Organization and sequence of the gene encoding the human acrosin-trypsin inhibitor (HUSI-II). Gene 123, 277-281 (1993).

18. Aebi, M., Horning, H., Padgett, R. A., Reiser, J. & Weissmann, C. Sequence requirements for splicing of higher eukaryotic nuclear pre-mRNA. Cell 47, 555-565 (1986).

19. Turki J. et al. Genetic polymorphisms of the beta-2 adrenergic receptor in nocturnal and non-nocturnal asthma: evidence that gly16 correlates with the noctural phenotype. J. Clin. Invest. 95, 1635-1641 (1995).

20. Chruscinski, A. J. et al. Targeted disruption of the 2 Adrenergic Receptor gene. J. Biol. Chem. 274, 16694-16700 (1999).

21. Yeo, J. P. et al. A new chromosomal protein essential for mitotic spindle assembly. Nature. 367, 288-291 (1994).

22. Oettgen, H., C., & Geha, R., S. IgE in asthma and atopy: cellular and molecular connections. J. Clin. Invest. 104, 829-835 (1999).

23. Huang, S. H. et al. Autosomal recessive retinitis pigmentosa caused by mutations in the alpha subunit of rod cGMP phosphodiesterase. Nat Genet. 11, 468-471.

24. Hastbacka, J. et al. The Diastrophic Dysplasia gene encodes a novel sulfate transporter: positional cloning by fine-structure linkage disequilibrium mapping. Cell. 78, 1073-1087.

25. Grimbacher, B et al. Genetic linkage of hyper-IgE syndrome to chromosome 4. Am. J. Hum. Genet. 65, 735-744 (1999).

26. Rheinwald, J. G. & Green, H. Serial cultivation of strains of human epidermal keratinocytes; the formation of keratinocytes colonies from single cells. Cell 6, 331-344 (1975).

27. Chomczynski, P. & Sacchi, N. Single-step method of RNA isolation by acid guanidinium thiocyanate-phenol-chloroform extraction. Anal. Biochem. 162, 156-159 (1987).

28. Ganguly, A., Rock M. J. & D. J., P. Conformation-sensitive gel electrophoresis for rapid detection of single base differences in double stranded PCR products and DNA fragments: evidence for solvent-induced bends in DNA heteroduplexes. Proc. Natl. Acad. Sci. 90, 10325-10329 (1993).

29. _Chien et al., Proc. Natl. Acadc. Sci. USA., 88, 9578-9582, 1991.

30. Tobe V. O., Taylor, S. L. and Nickerson D. A. Single well genotyping of diallelic sequence variations by a two-color ELISA-based oligonucleotide ligation assay. Nucleic Acids Res., 24(19), 3728-3732.

31. Schafer, A. J. and Hawkins, J. R. Nature Biotechnology. 1998; 16: 33-39.

32. Roskey, M. T. et. al. PNAS USA. 1996; 93: 4724-4729.

33. Shumaker, J. M. et. al. Hum. Mutat. 1996; 7: 346-354.

34. Pastinen, T. et. al. Genome Res. 1997; 7: 606-614.

35. Bunce M, O'Neill C, Barnardo M, al. e. Phototyping: Comprehensive DNA typing for HLA-A, B, C, DRB1, DRB3, DRB4, DRB5 and DQB1 by PCR with 144 primer mixes utlizing sequence-specific primers (PCR-SSP). Tissue Antigens 1995;50:23-31.

35. Underhill, P. A. et. al. PNAS USA. 1996; 93: 196-200.

36. Gilles, P. N. et. al. Nat. Biotech. 1999; 17: 365-370.

37. Superti-Furga, A. et al. Achondrogenesis type IB is caused by mutations in the diastrophic dysplasia sulfate transporter gene. Nat Genet. 12, 100-102.

1. A method of determining whether an individual is susceptible or predisposed to atopic disease which comprises screening the genome of the individual for the presence or absence of one or more polymorphic variants of the SPINK5 gene and/or screening for the expression of a variant LEKTI protein.

2. A method according to claim 1 wherein the at least one of the polymorphic variants is 1103 A→G or 1258 A→G.

3. A method according to claim 1 or claim 2 which additionally comprises determining the genotype of the said individual at one or more further polymorphic loci associated with atopic disease.

4. A method of determining whether an individual is susceptible or predisposed to atopic disease which comprises screening a tissue sample from the individual for the expression of a variant LEKTI protein.

5. A method according to claim 4 wherein the variant LEKTI protein has the amino acid substitution 368 Asn to Ser or 420 Glu to Lys.

6. A method according to claim 4 wherein the variant LEKTI protein is an alternatively spliced variant.

7. A method of determining the or any genetic basis of atopic disease in a patient previously diagnosed with such disease, which method comprises screening the genome of the individual for the presence or absence of one or more polymorphic variants of the SPINK5 gene.

8. A method according to claim 7 wherein at least one of the polymorphic variants is a single nucleotide polymorphism selected from 1103 A→G or 1258 A→G.

9. A method according to claim 7 or claim 8 which additionally comprises determining the genotype of the said individual at one or more further polymorphic loci associated with atopic disease.

10. A method according to any one of the preceding claims wherein the atopic disease is asthma, eczema or hay fever.

11. An isolated variant LEKTI polypeptide comprising the complete amino acid sequence illustrated in SEQ ID NO: 1 but having at least one of the following single amino acid substitutions:
Asn to Ser at position 39;
Asp to Asn at position 106;
Val to Ala at position 335;
Asn to Ser at position 368;
Asp to Asn at position 386;
Glu to Lys at position 420;
Arg to Ser at position 425;
Gly to Glu at position 519;
Arg to Lys at position 620;
Met to Ile at position 781;
Lys to Arg at position 822;
Glu to Asp at position 825;
Cys to Arg at position 930; or
His to Arg at position 972.

12. An isolated nucleic acid encoding a variant LEKTI protein aaccording to claim 11.

13. An isolated nucleic acid molecule comprising the complete nucleotide sequence illustrated in SEQ ID NO: 2 but having at least one of the following single nucleotide substitutions:
A substituted for G at position 56;
A substituted for G at position 81;
G substituted for A at position 116;
A substituted for G at position 316;
C substituted for T at position 1004;
G substituted for A at position 1103;
G substituted for A at position 1113;
A substituted for G at position 1156;
C substituted for T at position 1188;
T substituted for C at position 1257;
G substituted for A at position 1258;
T substituted for A at position 1275;
G substituted for A at position 1389;
A substituted for G at position 1556;
A substituted for C at position 1557;
T substituted for C at position 1659;
T substituted for C at position 1850;
A substituted for G at position 1859;
A substituted for G at position 2313;
A substituted for G at position 2343;
T substituted for C at position 2358;
T substituted for C at position 2368;
T substituted for C at position 2412;
G substituted for A at position 2465;
A substituted for G at position 2469;
G substituted for A at position 2472;
T substituted for G at position 2475;
C substituted for T at position 2788;
G substituted for A at position 2915; or
T substituted for C at position 3009
or the complement thereof.

14. A sense or antisense oligonucleotide comprising at least 15 consecutive nucleotides of a nucleic acid according to claim 13, including one of the specified single nucleotide substitutions.

15. Use of an oligonucleotide according to claim 14 as a hybridization probe or primer in a method to detect the presence of a variant SPINK5 allele containing the said single nucleotide substitution.

16. A kit for use in screening for human subjects for susceptibility or predisposition to atopic disease comprising at least one oligonucleotide probe or primer specific for a SPINK5 polymorphic variant associated with susceptibility or predisposition to atopic disease.

17. A kit according to claim 16 wherein the polymorphic variant associated with susceptibility or predisposition to atopic disease is 1103A→G or 1258A→G.

18. A genetic screening method for use in determining the carrier status of an individual for Netherton's syndrome or in diagnosing Netherton's syndrome in a patient, which method comprises screening the genome of the individual or patient for the presence of loss-of-function mutations in the SPINK5 gene.

19. A method according to claim 18 which comprises screening for at least one loss-of-function mutation generating a premature termination codon in the coding region of the SPINK5 gene.

20. A method according to claim 18 which comprises screening for at least one loss-of-function mutation which leads to alternative splicing of the mRNA encoded by the SPINK5 gene.

21. A method according to claim 18 which comprises screening for at least one loss-of-function mutation selected from the group consisting of: 81 G→A, 2258insG, 153delT, 238insG, 283−2A→T, 2468insA, 720insT, 1086delAT, 1888−1G→A, 2313G→A, 2369 C→T(R790X), 2468insA, 1038insG(A)₄, 1111C→T(R371X), 81+2T→A, 2459delA, 2259insA, 2240+1G→A, 81+5G→A, 1608−1GA, 2041delAG, 649C→T(R217X), 628C→T(R210X), 56G→A and 377delAT.

22. An isolated mutant SPINK5 allele containing a mutation selected from the group consisting of: 81 G→A, 2258insG, 153delT, 238insG, 283−2A→T, 2468insA, 720insT, 1086delAT, 1888−1G→A, 2313G→A, 2369 C→T(R790X), 1038insG(A)₄, 1111C→T(R371X), 81+2T→A, 2459delA, 2259insA, 2240+1G→A, 81+5G→A, 1608−1G→A, 2041delAG, 649C→T(R217X), 628C→T(R210X), 56G→A or 377delAT.

23. A nucleic acid probe which is specifically hybridizable to a mutant SPINK5 allele as defined in claim 22 but not to wild-type SPINK5.

24. A mutant LEKTI protein encoded by a mutant SPINK5 allele as defined in claim 22.

25. An isolated variant SPINK5 allele containing at least one polymorphic variant selected from the group consisting of:
82-31 A→G, 316 G→A, 475-86 G→C, 1011−12 C→T, 1093−26 C→T, 1093−10 A→G, 1103 A→G, 1156 G→A, 1188 T→C, 1258 A→G, 1221−50 G→A, 1389 A→G, 1557 C→A, 1607+47 C→T, 1659 CUT, 1821−47 T→G, 1888−54 G→A, 2241−27 TIC, 2313+31 C-G, 2313+48 G→A, 2358 C→T, 2412 C→T, 2475 G-T, 2740−59 G→A, 2915 A→G, 2965−46 T→C, 1257 C→T, 1113 A→G, 3009 C→T, 283−12T→A, 475−39A→G, 1302+19G→A, 1607+49delC, 1888−14T→C, 2313+21C-G, 2539−7T→G, 2667−22insT, 2965−8C→T, 3217+23T→C and 3217+23T→G.

26. A nucleic acid probe which is specifically hybridizable to a variant SPINK5 allele as defined in claim 25 but not to wild-type SPINK5.

27. A screening method for use in determining the carrier status of an individual for Netherton's syndrome or in diagnosing Netherton's syndrome in a patient which method comprises screening for expression of a protein product of the SPINK5 gene in a tissue sample from the individual or patient and thereby determining whether the protein is mutated and/or whether it is expressed at non-wild type levels.

28. A screening method for use in determining the carrier status of an individual for Netherton's syndrome or in diagnosing Netherton's syndrome in a patient, which method comprises determining the activity of any serine protease inhibitor encoded by the SPINK5 gene in a tissue sample from the individual or patient.

29. A method of determining the nature of the disease-causing mutation in a patient suffering or suspected of suffering from Netherton's syndrome, which method comprises comparing the nucleotide sequence of all or a part of the SPINK5 alleles carried by the patient with the wild type SPINK5 nucleotide sequence, wherein differences between the patient alleles and the wild-type alleles identify a disease-causing mutation.

30. A method according to claim 29 which comprises performing DNA amplification reactions on genomic DNA isolated from the patient using one or more primer-pairs specific to regions of the SPINK5 gene and analysing the products of the amplification reactions for any differences in size and/or nucleotide sequence compared to equivalent products amplified from known wild-type SPINK5 DNA using the same primer pairs.

31. A mutant SPINK5 allele which is identifiable using the method of claim 29 or claim 30.

32. A substance comprising a serine protease inhibitor having the amino acid sequence illustrated in SEQ ID NO: 1 or a functional fragment thereof for use in a method of treatment of the human body by therapy.

33. A substance according to claim 32 for use in the treatment of Netherton's syndrome.

34. A substance according to claim 32 for use in the treatment of atopic disease.

35. A method of treating atopic disease or Netherton's syndrome in a human patient which comprises administering to a patient in need thereof an effective amount of a medicament comprising a pharmaceutically active substance comprising a serine protease inhibitor having the amino acid sequence illustrated in SEQ ID NO: 1 or a functional fragment thereof and a pharmaceutically acceptable carrier, diluent or excipient.

36. A method of treating atopic disease or Netherton's syndrome in a human patient which comprises administering to a patient in need thereof an effective amount of a medicament comprising an expression vector suitable for directing expression of a serine protease inhibitor having the amino acid sequence illustrate in SEQ ID NO: 1 or functional fragment thereof in cells of the patient.

37. An expression vector comprising nucleic acid encoding a serine protease inhibitor comprising the amino acid sequence illustrated in SEQ ID NO: 1 or a functional fragment thereof operably linked to a promoter.

38. An expression vector according to claim 37 for use in a method of treatment of the human body by gene therapy.

39. A method of screening for compounds with potential pharmacological activity in the treatment of atopic disease or Netherton's syndrome, which method comprises:
determining the serine protease activity of a protein previously identified as a ligand of the LEKTI serine protease inhibitor in the presence and absence of a candidate compound, wherein compounds which are inhibitors of the serine protease activity of the ligand are scored as having potential pharmacological activity in the treatment of atopic disease or Netherton's syndrome.

40. A nucleic acid molecule having the complete nucleotide sequence illustrated in SEQ ID NO: 3 or a fragment thereof which retains promoter activity or tissue-specific transcriptional regulatory activity when assessed in a standard reporter gene assay.

41. A method of identifying a compound with potential pharmacological activity in the treatment of atopic disease or Netherton's syndrome, which method comprises:
providing a recombinant host cell containing a reporter gene expression construct comprising the promoter region of the human SPINK5 gene operably linked to a reporter gene;
contacting the host cell with a candidate compound; and
screening for expression of the reporter gene product.
